(19) United States
(12) Patent Application Publication
(10) Pub. No.: US 2022/0380738 A1
(43) Pub. Date: Dec. 1, 2022

(54) Programmable Cleavage of Double-Stranded DNA

(71) Applicant: New England Biolabs, Inc., Ipswich, MA (US)

(72) Inventors: Jurate Bitinaite, Rowley, MA (US); Rita Vaiskunaite, Peabody, MA (US); Vladimir Potapov, Auburndale, MA (US); Nathan Tanner, West Newbury, MA (US)

(73) Assignee: New England Biolabs, Inc., Ipswich, MA (US)

(21) Appl. No.: 17/335,500

(22) Filed: Jun. 1, 2021

(51) Int. Cl. C12N 9/14; C07K 14/195; C12N 15/11; C12N 15/90

(52) U.S. Cl. CPC C12N 9/14; C12Y 306/04012; C07K 14/195; C12N 15/11; C12N 15/907

(57) ABSTRACT

The present disclosure relates, according to some embodiments, to compositions, methods, systems, and kits for programmable endonucleolytic cleavage of DNA (e.g., ds DNA). For example, the in vitro activity of an Argonaute (e.g., a mesophilic Argonaute CbAgo from Clostridium butyricum) may be synchronized with DNA strand unwinding activity of a helicase (e.g., a nuclease deficient RecB^{exo-}C DNA helicase from E. coli) for a rapid and efficient cleavage of double-stranded DNA targets. Enzymatic properties of CbAgo and different aspects of ds DNA cleavage were thoroughly explored by adapting high-throughput capillary electrophoreses technique for monitoring CbAgo cleavage activity in concurrence with RecB^{exo-}C. The present disclosure shows that in the presence of RecB^{exo-}C, CbAgo can be programmed with guides to cleave any site of interest localized at up to 10 kb distance from the end of linear ds DNA at 37° C. temperature. CbAgo/RecB^{exo-}C can be programmed to generate DNA fragments flanked with unique single-stranded extensions suitable for seamless ligation with compatible DNA fragments. The present disclosure relates further the compositions, methods, systems, and kits for PRC-free assembly of linear DNA molecules by using CbAgo/RecB^{exo-}C programmable DNA endonuclease. The results presented here demonstrate that the combination of CbAgo and RecB^{exo-}C is currently an efficient mesophilic DNA-guided DNA-cleaving programmable endonuclease which can be used to prepare synthetic biology tools that require or benefit from sequence-specific nicking/cleavage of natural DNA at otherwise inaccessible locations.

### SEQUENCE LISTING STATEMENT

This disclosure includes a Sequence Listing submitted electronically in ascii format under the file name “NEB-440_ST25”. This Sequence Listing is incorporated herein in its entirety by this reference.

### BACKGROUND

Argonaute proteins have an ability to bind small single-stranded 5′-phosphorylated nucleic acids which provide base-pairing specificity for targeting complementary single-stranded targets. Eukaryotic Argonautes (eAgo) are essential components of certain RNA-induced gene silencing processes, in which eAgo associates with a single-stranded RNA guide to form an RNA-induced silencing complex (RISC). The RISC is then directed to the complementary sequence on mRNA molecules where Argonaute catalyzes the nucleolytic cleavage of single-stranded mRNA in a guide-specific manner, thus, resulting in a reduction of target gene expression. RISC also can interact with a variety of Argonaute-associated proteins to induce cleavage-independent mechanisms of gene regulation.

Despite the fact that prokaryotes lack RNA interference pathways, many bacterial and archaeal organisms also possess Argonaute proteins implying a different biological role and/or mechanism of action for these proteins within a cell. Multiple recent studies suggest that prokaryotic Argonautes in vivo function as defense systems against foreign genetic elements. Prokaryotic Agos (pAgos) represent a very diverse group of proteins and based on the presence or absence of the basic domains can be divided into two major groups—the short pAgos and the long pAgos. All known active pAgos belong to a long Ago group and, similar to eAgos, consist of four essential domains, N-terminal, PAZ, MID and PIWI. In contrast to eAgos, which use RNA guides to exclusively target RNA, different bacterial Agos have been shown to bind either RNA or DNA guides and to cleave either RNA or DNA targets, whereas some archaeal Agos exclusively utilize DNA guides for cleavage of DNA targets.

In addition to a guided cleavage of complementary targets, many pAgos exhibit non-specific nuclease activity when they are not associated with the guides. TtAgo co-purifies with DNA sequences that are preferentially derived from its own expression plasmid, but only if the Argonaute is catalytically active. Based on these and similar studies, the non-specific activity of pAgos was implicated in cellular function required for guide processing. While the physiological mechanism for DNA guide processing in vivo still remains ambiguous, the most recent study of mesophilic bacterial Argonaute CbAgo shows that CbAgo nucleolytic activity cooperates with cellular double-strand break repair machinery in generation of small DNAs that later can be used as guides by this Argonaute.

### SUMMARY

The present disclosure relates, in some embodiments, a non-naturally occurring composition comprising a helicase, a first Argonaute (e.g., a mesophilic Argonaute) bound to a first guide (e.g., an engineered guide), and optionally, a second Argonaute (e.g., a mesophilic Argonaute) bound to a second guide (e.g., an engineered guide). Each guide may be engineered or programmed to bind (e.g., base pair) with specific complementary sequences in a target polynucleotide. A non-naturally occurring composition may comprise a first Argonaute and the first guide at an Argonaute:guide molar concentration ratio of 2:1 to 1:2 or an Argonaute:guide molar concentration ratio equal to or lower than 1:1.4. A non-naturally occurring composition may comprise a second Argonaute and the second guide at an Argonaute:guide molar concentration ratio of 2:1 to 1:2 or an Argonaute:guide molar concentration ratio equal to or lower than 1:1.4. In some embodiments, a composition may further comprise a double-stranded polynucleotide (e.g., a target DNA). A double-stranded polynucleotide may comprise a nucleotide sequence complementary to a first guide and/or complementary to a second guide.

In some embodiments, a composition may comprise an Argonaute (e.g., a first Argonaute bound to a first guide and/or a second Argonaute bound to a second guide) selected from an Aquifex aeolicus Argonaute, an Aquifex aeolicus Argonaute, a Microsystis aeruginosa Argonaute, a Clostridium bartlettii Argonaute, an Exiguobacterium Argonaute, an Anoxybacillus flavithermus Argonaute, a Halogeometricum borinquense Argonaute, a Halorubrum lacusprofundi Argonaute, an Aromatoleum aromaticum Argonaute, a Synechococcus Argonaute, a Clostridium butyricum Argonaute (CbAgo), a Clostridium disporicum Argonaute (CdAgo), a Clostridium perfringens Argonaute (CpAgo), a Clostridium sartagoforme Argonaute (CsAgo), a Clostridium saudiense Argonaute (CaAgo), an Intestinibacter bartlettii Argonaute (IbAgo) and, in each case, homologues thereof (e.g., Argonautes having at least 90% amino acid sequence identity thereto). For example, an Argonaute (e.g., a first Argonaute bound to a first guide) may be CaAgo, CbAgo, CdAgo, CpAgo, CsAgo or IbAgo and the Argonaute bound to the second guide is independently CaAgo, CbAgo, CdAgo, CpAgo, CsAgo or IbAgo. A helicase may be selected from an EcoRecQ DNA helicase from Escherichia coli, a CpeRecQ from Clostridium perfringens, a Cbu RecQ from Clostridium butyricum, a DNA helicase from T4-like bacteriophage (e.g., T4 gp41, T4 gp41 associated with T4 gp59, T4 UvsW, T4 Dda and Slur07 Dda), a T7 bacteriophage gp4 DNA helicase, RecBCD-family helicases from Escherichia coli, a modified RecBCD helicase (e.g., RecB^{exo-} helicase, RecB^{exo-}C, RecB^{exo-}CD, RecΔB, RecΔBC, RecΔBCD), a UvrD/PcrA family helicase (e.g., E. coli EcoUvrD), an E. coli Rep, an M. tuberculosis PcrA, an M. leprae PcrA, and an Escherichia coli Tra helicase. Each guide (e.g., engineered guide) may be independently (of other guides in a composition) 12-60 nucleotides in length.

The present disclosure relates, in some embodiments, to methods of forming a double strand break in a double-stranded polynucleotide at a target position in the polynucleotide. For example, a method may comprise contacting (a) a double-stranded polynucleotide having a first target sequence on a first strand of the polynucleotide and a second target sequence on the opposite strand, (b) a helicase, (c) an Argonaute with a first bound guide having a sequence complimentary to the first target sequence, and (d) an Argonaute with a second bound guide having a sequence complimentary to the second target sequence under conditions that permit hybridization of complimentary sequences and cleavage of the first strand by the (c) Argonaute and cleavage of the second strand by the (d) Argonaute to produce a double strand break in the polynucleotide. A method of forming a double-strand break may be performed at a wide range of suitable temperatures. For example, a method of forming a double strand break in a double-stranded polynucleotide at a target position in the polynucleotide may include contacting reaction components disclosed above and herein at a temperature of 25° C. to 45° C. A method of forming a double strand break in a double-stranded polynucleotide may comprise forming at least a first fragment of the polynucleotide and a second fragment of the polynucleotide. For example, the number of fragments of the starting polynucleotide formed may be a function of the number and position of the sequence(s) complimentary to the guide(s). A first guide may have a sequence complementary to the first target sequence in the polynucleotide. A method of forming a double strand break in a double-stranded polynucleotide may comprise forming one or more fragments of the polynucleotide with one or more such fragments comprising a blunt end, an overhang from 1 to 50 nucleotides in length (which overhang may be a 5′ or a 3′ overhang), and/or an overhang from 51 to 100 nucleotides in length (which overhang may be a 5′ or a 3′ overhang).

A method may comprise, for example, (a) contacting a helicase, an Argonaute, a guide DNA bound to the Argonaute, and a polynucleotide comprising a target sequence that is complementary to at least part of the guide DNA, to produce a reaction mix; and/or (b) incubating the reaction mix at a temperature of 25° C. to 45° C., wherein the nucleic acid is cleaved. Contacting, in this context, may further comprise contacting the helicase, the Argonaute, the guide DNA bound to the Argonaute, the polynucleotide, a second Argonaute, and a second guide bound to the second Argonaute, wherein the polynucleotide further comprises a second target sequence that is complementary to at least part of the second guide DNA.

### BRIEF DESCRIPTION OF THE FIGURES

FIG. 1 shows the DNA-guided cleavage activity on ss DNA at 37° C. and 65° C. temperature displayed by example Argonaute proteins. Argonaute proteins were synthesized in vitro using PURExpress protein synthesis kit (New England Biolabs Inc., Ipswich, Mass., USA). Reaction conditions are described in Example 6. The name of the pAgo used is shown above the respective lane. Bacterial hosts and NCBI Gene bank Accession numbers are listed in Table 3.

FIG. 2 shows a comparison of Clostridia Argonautes for cleavage efficiency on ss DNA target. Reactions were performed at 5:5:2 Ago:Guide:Target molar concentration ratio (125 nM Ago, 125 nM 17 nt 5′-phosphorylated guide, 50 nM 5′-FAM labeled ss DNA). Reactions were incubated at 37° C. and samples were removed at indicated time points. DNA cleavage was terminated by addition of 50 mM EDTA. DNA samples at a 4 nM final concentration were resolved by Capillary electrophoresis (CE) as described in Example 6. Bacterial hosts of Ago proteins used in this study: C. butyricum (CbAgo), C. disporicum (CdAgo), C. perfringens (CpAgo), C. sartagoforme (CsAgo), C. saudiense (CaAgo), and I. bartlettii (IbAgo).

FIG. 3A and FIG. 3B show guide-specific cleavage of single-stranded DNA by CbAgo loaded with different guides. FIG. 3A shows arrangement of thirteen single-stranded guides (SEQ ID NOS: 30-42, respectively) on a 53 nt 5′-FAM labeled single-stranded DNA substrate (SEQ ID NO: 11). 5′-phosphorylated guides of 21-nt in length were designed to hybridize with a 53 nt ss DNA substrate at sequences that are shifted by one nucleotide with respect to each other. The names of the guides are shown on the right next to the respective guide. Within a guide sequence, the 10^{th} nucleotide starting from a 5′-phosphate is underlined to mark a position that aligns with the CbAgo cut site on the target sequence. Arrow on the left shows the target cleavage position of CbAgo loaded with guide T1. Cleavage position of CbAgo loaded with each subsequent guide is shifted by one nucleotide along the substrate DNA, and the arrow on the right marks the cleavage position of CbAgo loaded with the last guide T3. FIG. 3B shows a comparison of CbAgo cleavage efficiency when it is loaded with different guides. Cleavage reaction settings are described in EXAMPLE 6. For each CbAgo/guide complex, the percentage of cleaved DNA target was quantified after 1-, 4- and 16-minute incubation at 37° C.

FIG. 4A and FIG. 4B show data demonstrating DNA unwinding activity of four commercially available DNA helicases. FIG. 4A shows a schematic overview of a helicase activity assay which is based on the creation of DpnI restriction endonuclease cleavage site only if helicase exhibits DNA unwinding activity. FIG. 4B shows unwinding activity of tested DNA helicases. LANE 1 shows untreated DNA substrate. LANE 2 shows DNA substrate treated with DpnI restriction endonuclease. LANE 3 shows DNA unwinding activity of EcoRecQ DNA helicase purchased from Abcam (Abcam, Inc., Cambridge, Mass., USA) LANE 4 shows DNA unwinding activity of T4 helicase purchased from McLab (McLab, Inc., South San Francisco, Calif., USA). LANE 5 shows DNA unwinding activity of T4 gp41 helicase purchased from GoldBio (GoldBio Inc., St. Louis Mo., USA). LANE 6 shows DNA unwinding activity of EcoUvrD helicase purchased from MyBioSource (MyBioSource, Inc. San Diego, Calif., USA).

FIG. 5A and FIG. 5B show data demonstrating guide-specific cleavage of double-stranded DNA by CbAgo in the presence of commercial EcoRecQ and Slur07 Dda DNA helicases (T4 DNA helicase from McLab). FIG. 5A shows a schematic overview of an Argonaute activity assay on ds DNA that combines duplex DNA unwinding by helicase and single-strand cleavage by CbAgo. FIG. 5B demonstrates that CbAgo loaded with a 17 nt guide cleaves double-stranded DNA in the presence of DNA helicase. LANE 1 shows 5′-FAM labeled 30 bp long ds DNA substrate tailed with a 30 nt long forked end. LANE 2 demonstrates that CbAgo/guide complex alone has no activity on ds DNA. LANE 3 demonstrates that CbAgo cleaves ds substrate after EcoRecQ helicase unwinds DNA duplex. LANE 4 demonstrates that CbAgo cleaves ds substrate after Slur07 Dda helicase unwinds DNA duplex.

FIG. 6 shows a comparison of DNA unwinding activity between three RecQ DNA helicases. Bacterial hosts of DNA helicases used here: CpeRecQ from Clostridium perfringens (WP_011590145.1), Cbu RecQ from Clostridium butyricum (WP_003411240.1) and EcoRecQ from Escherichia coli (WP_096324295.1).

FIG. 7A and FIG. 7B demonstrates CbAgo activity in the presence of CpeRecQ, EcoRecQ and Slur07Dda DNA helicases on 50-60 bp ds substrates that have different end structures. FIG. 7A shows the structure of each DNA substrate. Substrate 1 has a 50 bp duplex region extended with a 10 nt long fork structure. Substrate 2 has a 60 bp duplex region extended with a 10 nt long single-stranded sequence on a 3′-terminus. Substrate 3 has a 60 bp duplex region extended with a 10 nt long single-stranded sequence on a 5′-terminus. Substrate 4 is a 60 bp long blunt-ended DNA. FIG. 7B shows data demonstrating CbAgo activity on each DNA substrate either in the presence of a single DNA helicase Slur07Dda, EcoRecQ or CpeRecQ helicase, or in the presence of two helicase combinations, EcoRecQ+Slur07Dda or CpeRecQ+Slur07Dda. 50 nM of each DNA substrate was cleaved with CbAgo in the presence of the indicated DNA helicase for 1 hour at 37° C.

FIG. 8A and FIG. 8B show data demonstrating CbAgo activity in the presence of CpeRecQ, EcoRecQ and Slur07 Dda DNA helicases on 80 bp ds substrates that have different end structures. FIG. 8A shows the structure of each DNA substrate. Substrate 1 has an 80 bp duplex region extended with a 10 nt fork structure. Substrate 2 has an 80 bp duplex region extended with a 10 nt single-stranded sequence on a 3′-terminus. Substrate 3 has an 80 bp duplex region extended with a 10 nt long single-stranded sequence on a 5′-terminus. Substrate 4 is an 80 bp blunt-ended DNA. FIG. 8B shows data that demonstrating CbAgo activity on each DNA substrate either in the presence of a single DNA helicase: Slur07 Dda, EcoRecQ or CpeRecQ helicase, or in the presence of two helicase combinations, EcoRecQ+Slur07Dda or CpeRecQ+Slur07Dda. 50 nM of each DNA substrate was cleaved with CbAgo in the presence of the indicated DNA helicase for 1 hour at 37° C.

FIG. 9 shows data demonstrating DNA unwinding activity of RecB^{exo-} and RecB^{exo-}C DNA helicases. First panel shows reactions were carried out in the absence (LANE 1) or in the presence of 2.5, 1.25, 0.63 and 0.31 μM RecB^{exo-} (LANES 2-5, respectively). Next panel show reactions were carried out in the absence (LANE 1) or in the presence of 1, 0.5, 0.25 and 0.125 μM RecB^{exo-}C (LANES 2-5, respectively).

FIG. 10A and FIG. 10B show a 5′FAM-labeled strand cleavage after a 322 bp ds DNA was treated in the presence of RecB^{exo-}C with CbAgo loaded with a single guide. FIG. 10A shows arrangement of thirteen guides (SEQ ID NOS: 30-42) on the 322 bp 5′-FAM labeled double-stranded DNA substrate (SEQ ID NO: 11 and 84). 5′-phosphorylated guides were designed to hybridize with the 5′-FAM labeled strand at sequences that were shifted by one nucleotide with respect to each other. The names of the guides are shown on the right next to the respective guide. Within a guide sequence, the 10^{th} nucleotide starting from a 5′-phosphate is underlined to mark a position that aligns with the CbAgo cut site on the targeted DNA strand. Arrow on the left shows the cleavage position on DNA strand by CbAgo loaded with a guide T1. Cleavage position of CbAgo loaded with each consecutive guide is shifted by one nucleotide along the substrate DNA, and the arrow on the right marks the cleavage position of CbAgo loaded with a guide T3. FIG. 10B shows comparison of CbAgo cleavage efficiency when it is loaded with indicated guides. Cleavage reaction settings are described in EXAMPLE 7. For each CbAgo/guide complex, the fraction of cleaved DNA target was quantified after 4-, 16- and 64-minute incubation at 37° C.

FIG. 11A and FIG. 11B show 5′ROX-labeled strand cleavage after 322 bp ds DNA was treated in the presence of RecB^{exo-}C with CbAgo loaded with a single guide. FIG. 11A shows arrangement of four guides—B1 (SEQ ID NO: 43), B2 (SEQ ID NO: 44), B3 (SEQ ID NO: 45), or B4 (SEQ ID NO: 46)—for targeting of a 5′ROX-labeled DNA strand (SEQ ID NO: 85) on the 322 bp DNA substrate. Within a guide sequence, the 10^{th} nucleotide starting from a 5′-phosphate is underlined to mark a position that aligns with the CbAgo cut site on the targeted DNA strand. Arrows show CbAgo cleavage positions on the 5′-ROX labeled DNA strand for each respective guide. FIG. 11B shows comparison of CbAgo cleavage efficiency when it is loaded with indicated guides. Cleavage reaction settings are described in EXAMPLE 7. For each CbAgo/guide complex, the fraction of cleaved DNA target was quantified after 2-, 4- and 16-minute incubation at 37° C.

FIG. 12A, FIG. 12B, FIG. 12C, and FIG. 12D show time course results of double-strand cleavage after 5′-FAM/ROX labeled 322 bp DNA was cleaved with CbAgo loaded with either T2+B1 guide pair or T1-C2+B4 guide pair. FIG. 12A shows T2 (SEQ ID NO: 38) and B1 (SEQ ID NO: 43) guide sequence alignment with the complementary DNA strands (SEQ ID NO: 87 and SEQ ID NO: 86) of 322 bp DNA. CbAgo loaded with guides T2+B1 generate cleavage products flanked with 5 nt-long 3′-single-stranded extensions. FIG. 12B shows time course data demonstrating the concurrent cleavage of both DNA strands by CbAgo in the presence of RecB^{exo-}C helicase. Line with open squares shows 5′-FAM labeled DNA strand cleaved by CbAgo/T2 complex. Line with open circles shows 5′-ROX labeled DNA strand cleaved by CbAgo/B1 complex. FIG. 12C shows guide T1-C2 (SEQ ID NO: 33) and B4 (SEQ ID NO: 46) sequence alignment with the complementary DNA strands (SEQ ID NO: 86 and SEQ ID NO: 87) of 322 bp DNA. CbAgo loaded with guides T1-C2+B4 generate cleavage products flanked with 5 nt-long 5′-single-stranded extensions. FIG. 12D shows time course data demonstrating the concurrent cleavage of both DNA strands by CbAgo in the presence of RecB^{exo-}C helicase. Line with open squares shows 5′-FAM labeled DNA strand cleaved by CbAgo/T1-C2 complex. Line with open circles shows 5′-ROX labeled DNA strand cleaved by CbAgo/B4 complex.

FIG. 13A and FIG. 13B show double-strand cleavage of 5′-FAM/ROX labeled 322 bp ds DNA by CbAgo loaded with thirteen different guide pairs which produce cleavage products flanked with 3′-single-stranded extensions. A 5′-ROX labeled DNA strand is cleaved by CbAgo/B1 complex, whereas a 5′-FAM labeled strand is cleaved by CbAgo loaded with different guides. FIG. 13A shows arrangement of guide sequences (SEQ ID NOS:30-42) on DNA strands (SEQ ID NOS: 11 and 85). Guide B1 (SEQ ID NO: 43) is complementary to the 5′-ROX labeled strand and arrow indicates CbAgo/B1 cleavage position. Thirteen guides complementary to the 5′FAM-labeled DNA strand have nucleotide sequences shifted by one nucleotide with respect to each other. Within a guide sequence, the 10^{th} nucleotide starting from a 5′-phosphate is underlined to mark a position that aligns with the CbAgo cut site on targeted DNA strand. Arrow indicates the cleavage position of CbAgo/T1 complex. Cleavage position of CbAgo loaded with each consecutive guide is shifted by one nucleotide to the right along the 5′FAM-labeled DNA strand. FIG. 13B shows data demonstrating efficacy of double-strand cleavage by CbAgo loaded with the indicated guide pairs. Reactions were performed with CbAgo/guide complexes assembled with 125 nM CbAgo and 250 nM guide for a molar concentration ratio of 1:2 Ago:Guide. In cleavage reactions, two opposing CbAgo/guide complexes were combined with 50 nM DNA substrate in the presence of 250 nM RecB^{exo-}C helicase for 16 minutes at 37° C. and the percentage of cleaved DNA was quantified for each individual strand. The length of 3′ single-stranded extensions on cleavage products generated by CbAgo programmed with the respective guide pair is shown at the bottom.

FIG. 14A, FIG. 14B and FIG. 14C show double-strand cleavage of 5′-FAM/ROX labeled 322 bp ds DNA by CbAgo loaded with different guide pairs which produce cleavage products flanked with 5′-single-stranded extensions. 5′-ROX labeled DNA strand is cleaved by CbAgo/B4 complex, whereas a 5′-FAM labeled strand is cleaved by CbAgo loaded with different guides. FIG. 14A shows arrangement of guide sequences (SEQ ID NOS: 30-42) on DNA strands (SEQ ID NOS: 11 and 85). Guide B4 (SEQ ID NO: 46) is complementary to the 5′-ROX labeled strand and arrow indicates CbAgo/B4 cleavage position. Thirteen guides complementary to the 5′-FAM labeled DNA strand have nucleotide sequences shifted by one nucleotide with respect to each other. Within a guide sequence, the 10^{th} nucleotide starting from a 5′-phosphate is underlined to mark a position that aligns with the CbAgo cut site on the targeted DNA strand. Arrow indicates the cleavage position of CbAgo/T3 complex. Cleavage position of CbAgo loaded with each consecutive guide is shifted by one nucleotide to the left along the 5′FAM-labeled DNA strand. FIG. 14B shows data demonstrating efficacy of double-strand cleavage at 125 nM CbAgo and 250 nM guide (a CbAgo:guide molar concentration ratio of 1:2). In these reactions, two opposing CbAgo/guide complexes were combined with 50 nM DNA substrate in the presence of 250 nM RecB^{exo-}C helicase for 64 minutes at 37° C. and percentage of cleaved DNA was quantified for each individual strand. The length of 5′ single-stranded extensions on cleavage products generated by CbAgo programmed with the respective guide pair is shown at the bottom. FIG. 14C shows data demonstrating efficacy of double-strand cleavage at a 1:1 CbAgo:Guide molar concentration ratio (125 nM CbAgo:125 nM guide). In these reactions, two opposing CbAgo/guide complexes were combined with 50 nM DNA substrate in the presence of 250 nM RecB^{exo-}C helicase for 32 minutes at 37° C. and percentage of cleaved DNA was quantified for each individual strand.

FIG. 15 shows efficiency of double-strand cleavage of 5′-FAM/ROX labeled 322 bp ds DNA at different CbAgo:guide molar concentration ratios. CbAgo was loaded with guides T1-A1 and B4 at a 1:2 (125:250 nM), 1:1.8 (125:225 nM), 1:1.6 (125:200 nM), 1:1.4 (125:175 nM), 1:1.2 (125:150 nM) and 1:1 (125:125 nM) molar concentration ratios. In these reactions, the opposing CbAgo/guide complexes were combined with 50 nM DNA substrate in the presence of 250 nM RecB^{exo-}C helicase for 16 minutes at 37° C. and the percentage of cleaved DNA was quantified for each individual strand.

FIG. 16A, FIG. 16B, FIG. 16C, FIG. 16D, FIG. 16E, and FIG. 16F show CbAgo acting as a programmable double-stranded DNA endonuclease on long linear DNA. Reactions contained 0.2 μg of linear DNA, 5 mM ATP and CbAgo/guide at a 0.125:0.25 μM concentration ratio in 1X CutSmart buffer. The CbAgo cleavage was carried out at 37° C. for 1 hour either in the absence (LANE 1) or in the presence of 0.25 μM RecB^{exo-}C(LANE 2). The reactions were stopped by treatment with 8 units of Proteinase K for 30 minutes at 25° C. CbAgo cleavage products were column purified and separated by gel electrophoresis on 1.2% agarose gel. (S) indicates φX174 DNA linearized by the respective restriction endonuclease. (P) indicates linear DNA is cleaved by CbAgo loaded with the respective guide pair.

FIG. 16A shows a circular map of φX174 DNA with restriction sites and guide positions. FIG. 16B shows four locations of linearized φX174 DNA targeted with CbAgo loaded with four different sets of guide pairs, T2+B1, XhoT+XhoB, StuT+StuB and SapT+SapB. PANEL I shows SspI-linearized DNA was cleaved with CbAgo loaded with guides T2 and B1. LANE 3, SspI+SapI digested DNA. PANEL II shows XhoI-linearized DNA was cleaved with CbAgo loaded with guides XhoT and XhoB. PANEL III shows StuI-linearized DNA was cleaved with CbAgo loaded with guides StuT and StuB. PANEL IV shows SapI-linearized DNA was cleaved with CbAgo loaded with guides SapT and SapB.

FIG. 16C shows a circular map of pAd2_BsaBI DNA (22.1 kb) with AscI restriction site and guide positions. FIG. 16D shows AscI-linearized pAd2_BsaBI DNA (LANE 1) was cleaved with CbAgo loaded with guides AdB-450T and AdB450B (LANE 2).

FIG. 16E shows a circular map of pAd2_AvrII DNA (25.1 kb) with SrfI restriction site and guide positions. FIG. 16F shows SrfI-linearized pAd2_AvrII DNA (LANE 1) was cleaved with CbAgo loaded with guides AdA-20860T and AdA-20860B (LANE 2).

FIG. 17A and FIG. 17B show a PCR-free method for a seamless assembly of DNA fragments using CbAgo and RecB^{exo-}C. FIG. 17A shows a schematic overview of seamless DNA assembly. CbAgo/guide cleavage was carried out in the presence of RecB^{exo-}C DNA helicase as described in EXAMPLES 7 and 9. The enzymes were inactivated by treatment with Proteinase K for 30 min at 25° C. and Monarch® PCR purification kit (New England Biolabs, Inc., Ipswich, Mass., USA) was used to eliminate short cleavage products. The removed nucleotide sequences were synthetically combined into a double-stranded Bridge oligo. Synthetic Bridge oligo was then sequentially ligated to DNA1 and DNA 2 through complementary single-stranded extensions to create a directionally assembled recombinant DNA. FIG. 17B shows CbAgo cleavage products and ligation products were analyzed by capillary electrophoreses (CE). The solid traces show ROX-labeled strand of uncut DNA 1 and DNA 2, the ROX-labeled strand of 15 bp and 278 bp CbAgo cleavage products and the ROX-labeled strand of 632 bp ligation product, respectively. The dashed traces show FAM-labeled strand of uncut DNA 1 and DNA 2, the FAM-labeled strand of 309 bp and 14 bp CbAgo cleavage products and the FAM-labeled strand of 346 bp and 632 bp ligation products, respectively. Anticipated ligation products are marked with an asterisk (*).

FIG. 18A, FIG. 18B, FIG. 18C and FIG. 18D demonstrates CbAgo activity on double-stranded DNA in the presence of RecABC DNA helicase alone or jointly with CpeRecQ, EcoRecQ or Slur07 Dda DNA helicases. FIG. 18A shows data demonstrating cleavage activity of CbAgo loaded with guide B12 on 80 bp blunt-ended DNA substrate. CbAgo cleavage reactions were incubated for 1 hour at 37° C. in the absence of DNA helicase (LANE 2) or in the presence of indicated DNA helicases (LANES 3-6). LANE 1 shows untreated DNA substrate. FIG. 18B shows data demonstrating cleavage activity of CbAgo loaded with guide T2 on 239 bp blunt-ended DNA substrate. CbAgo cleavage reactions were incubated for 1 hour at 37° C. in the absence of DNA helicase (LANE 2) or in the presence of indicated DNA helicases (LANES 3-5). LANE 1 shows untreated DNA substrate. FIG. 18C shows data demonstrating cleavage activity of CbAgo loaded with guide T2 on 619 bp blunt-ended DNA substrate. CbAgo cleavage reactions were incubated for 1 hour at 37° C. in the absence of DNA helicase (LANE 2) or in the presence of indicated DNA helicases (LANES 3-5). LANE 1 shows untreated DNA substrate. FIG. 18D shows data demonstrating double-strand cleavage of 239 bp and 619 bp DNA by CbAgo in the presence of RecΔBC+CpeRecQ helicases. CbAgo was programmed with guide pair T2+B1 and DNA substrate was cleaved for 1 hour at 37° C. The percentage of cleaved DNA was quantified for each individual strand.

FIG. 19A and FIG. 19B show double-strand cleavage of 5′-FAM/ROX labeled 322 bp ds DNA by CbAgo/T2+B1 in the presence of RecB^{exo-}C at 25° C., 30° C., 37° C., 42° C. or 50° C. temperature. FIG. 19A shows data demonstrating cleavage of 5′-FAM labeled DNA strand by CbAgo loaded with guide T2. FIG. 19B shows data demonstrating cleavage of 5′-ROX labeled DNA strand by CbAgo loaded with guide B1.

FIG. 20A, FIG. 20B, FIG. 20C, FIG. 20D, FIG. 20E, FIG. 20F, FIG. 20G, and FIG. 20H show comparisons of CbAgo/RecB^{exo-}C activity on unmodified, 5mC-modified, 5hmC-modified and 5ghmC-modified DNA substrates. DNA substrates were generated by PCR using dNTPs containing either dCTP to generate an unmodified substrate (C) or 5-methyl-dCTP to generate a 5-methylcytosine-containing substrate (mC) or 5-hydroxymethyl-dCTP to generate a 5-hydroxymethylcytosine containing substrate (hmC). hmC substrate was glucosylated using T4-BGT glucosyltransferase (New England Biolabs, Inc., Ipswich, Mass., USA) in the presence of UDP-Gluc to generate a beta-glucosyl-5-hydroxymethylcytosine containing substrate (ghmC).

FIG. 20A and FIG. 20B show cleavage activity of CbAgo loaded with guide B2. FIG. 20A shows arrangement of guide B2 (SEQ ID NO: 44) on 322 bp 5′-ROX labeled DNA substrate (SEQ ID NOS: 88 and 89). Guide B2 is complementary to the 5′-ROX labeled strand and arrow indicates CbAgo/B2 cleavage position. The cytosine residues which were modified in mC, hmC and ghmC substrates are shown in bold. The modified cytosines which complement dG residues within the guide B2 sequence are underlined. FIG. 20B shows CbAgo/B2 cleavage of four DNA substrates over time.

FIG. 20C and FIG. 20D show cleavage activity of CbAgo loaded with guide B3. FIG. 20C shows arrangement of guide B3 (SEQ ID NO: 45) on 322 bp 5′-ROX labeled DNA substrate (SEQ ID NOS: 90 and 91). Guide B3 is complementary to the 5′-ROX labeled strand and arrow indicates CbAgo/B3 cleavage position. The cytosine residues which were modified in mC, hmC and ghmC substrates are shown in bold. The modified cytosines which complement dG residues within the guide B3 sequence are underlined. FIG. 20D shows CbAgo/B3 cleavage of four DNA substrates over time.

FIG. 20E and FIG. 20F show cleavage activity of CbAgo loaded with guide T3. FIG. 20E shows arrangement of guide T3 (SEQ ID NO: 42) on 322 bp 5′-FAM labeled DNA substrate (SEQ ID NOS: 89 and 90). Guide T3 is complementary to the 5′-FAM labeled strand and arrow indicates CbAgo/T3 cleavage position. The cytosine residues which were modified in mC, hmC and ghmC substrates are shown in bold. The modified cytosines which complement dG residues within the guide T3 sequence are underlined. FIG. 20F shows CbAgo/T3 cleavage of four DNA substrates over time.

FIG. 20G and FIG. 20H shows cleavage activity of CbAgo loaded with guide T2-T2. FIG. 20G shows arrangement of guide T2-T2 (SEQ ID NO: 39) on 322 bp 5′-FAM labeled DNA substrate (SEQ ID NOS: 92 and 93). Guide T2-T2 is complementary to the 5′-FAM labeled strand and arrow indicates CbAgo/T3 cleavage position. The cytosine residues which were modified in mC, hmC and ghmC substrates are shown in bold. The modified cytosines which complement dG residues within the guide T2-T2 sequence are underlined. FIG. 20H shows CbAgo/T2-T2 cleavage of four DNA substrates over time.

FIG. 21A, FIG. 21B and FIG. 21C demonstrate CbAgo cleavage of ds DNA after strands were denatured for 10 minutes at 85° C. in the presence of 20% Formamide. FIG. 21A shows time course of CbAgo/T2+B1 cleavage of 50 nM 322 bp FAM/ROX-labeled DNA. CbAgo was loaded with guides at 125:125 nM CbAgo:Guide molar concentration ratio. FIG. 21B shows time course of CbAgo/T2+B1 cleavage of 50 nM 619 bp FAM/ROX-labeled DNA. CbAgo was loaded with guides at 125:125 nM CbAgo:Guide molar concentration ratio. FIG. 21C shows cleavage of 50 nM 619 bp ROX-labeled DNA by CbAgo/T2+B1 loaded at 125:125 nM, 250:250 nM and 500:500 nM CbAgo:Guide molar concentration ratio.

### DETAILED DESCRIPTION

The present disclosure relates, in some embodiments, to compositions, methods, systems, and kits that modify argonaute's use of single-stranded DNA guides for sequence-specific cleavage of complementary DNA targets to yield pAgos adapted to serve as programmable DNA endonucleases. In some embodiments, cleavage may occur at a physiological temperature (e.g., at temperatures from 25° C. to 45° C.) optionally, in the presence of a helicase and/or a chemical agent. Examples of chemical agents include alkali, dimethylsulfoxide (DMSO), and formamide. Conditions may otherwise to permit or favor cleavage of and/or to destabilize Watson-Crick bonding in double stranded DNA, for example, by contacting the substrate DNA with media having a low or high ionic strength, with air, and/or with glass.

The CRISPR/Cas9 system is a widely used enzymatic tool for programmable DNA cleavage. Cas9 nuclease is programmed with a single RNA guide and can invade ds DNA structure. It generates double-strand breaks at a guide-specific DNA target. Also, the system functions at physiological temperatures, so it had been successfully adapted for genome editing in vivo (Wang H et al., Annu. Rev. Biochem. 2016, 85:227-264).

In contrast to CRISPR-Cas9, mesophilic pAgos have been observed to act poorly on double-stranded targets due to their poor ability to invade duplex DNA. Characterized pAgos that function at 30-75° C. temperatures show low levels of endonucleolytic activity on ds DNA preferentially targeting negatively supercoiled plasmids and/or DNA sections with low G/C content. These preferences are consistent with DNA duplex destabilization aiding pAgos to access targets on ds DNA. At a temperature that causes thermal DNA denaturation (e.g., >87° C.), hyperthermophilic pAgos (e.g., PfAgo from the archeon Pyrococcusfuriosus) work in vitro as programmable DNA-guided DNA-cleaving endonucleases. After DNA melting takes place, the double-strand cleavage by PfAgo proceeds by way of two independent strand-nicking events catalyzed by two PfAgo monomers, each loaded with a guide complementary to one DNA strand.

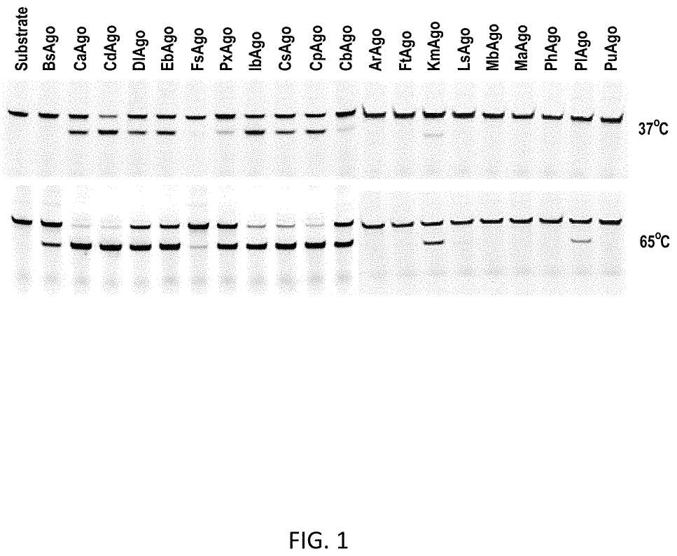

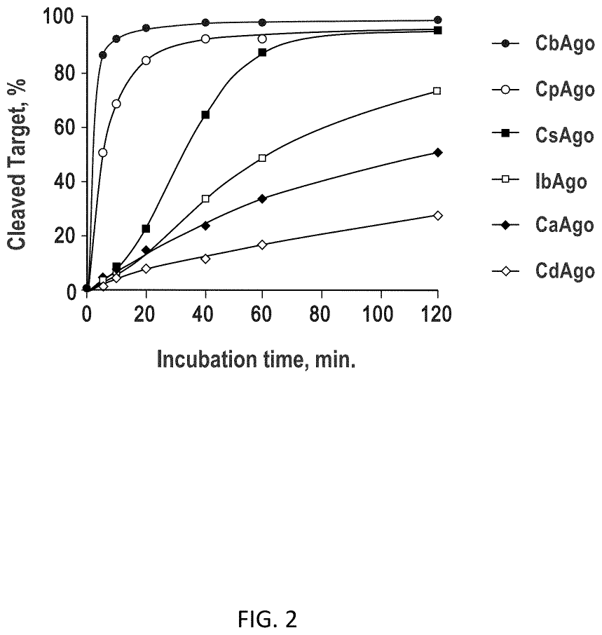

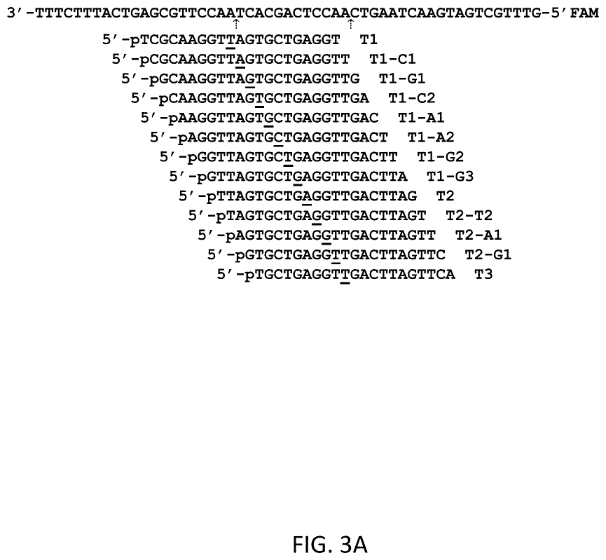

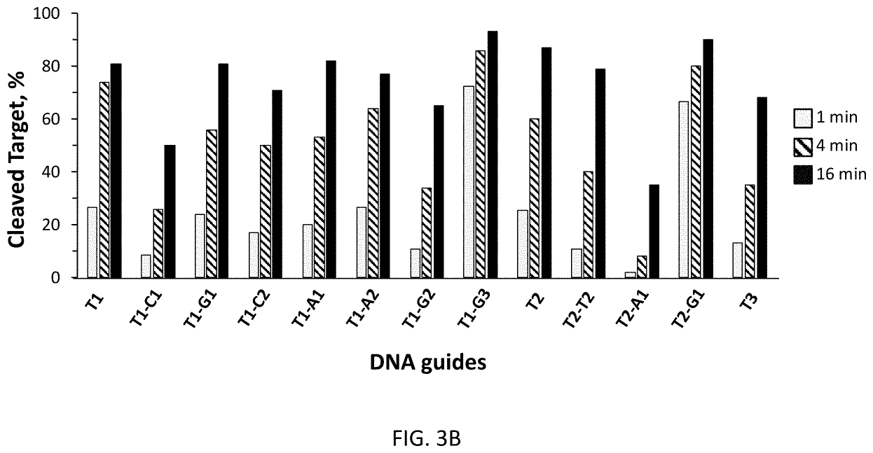

Putative pAgo proteins were screened for cleavage activity at 37-65° C. temperature and candidates were identified that are active at 37° C. (FIG. 1). Selected mesophilic argonautes originating from Clostridia class bacteria were biochemically characterized (FIG. 2, TABLE 4). CbAgo was shown to efficiently cleave single-stranded targets at 37° C. (FIG. 3). However, CbAgo activity on double-stranded targets was found insignificant under the conditions tested. A variety of proteins which have an ability either to intercalate into ds DNA or to stabilize ss DNA regions during various cell processes, such as DNA replication, DNA recombination and DNA repair, were tested in combination with CbAgo for ds DNA cleavage at 37° C. Tested accessory proteins were not capable of increasing CbAgo cleavage activity on ds DNA targets at 37° C. under the conditions tested (EXAMPLE 13).

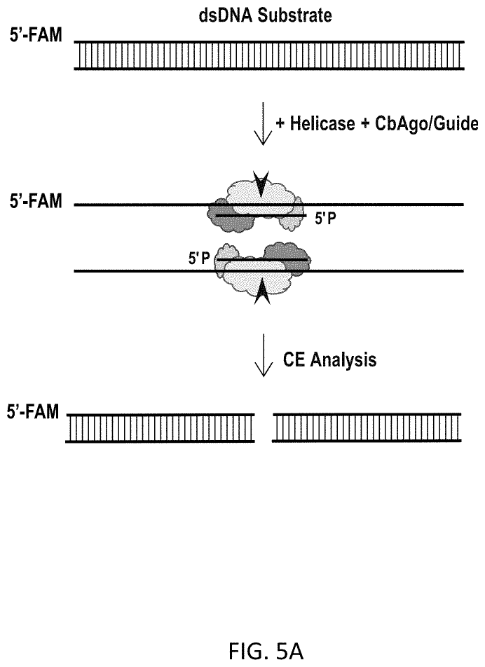

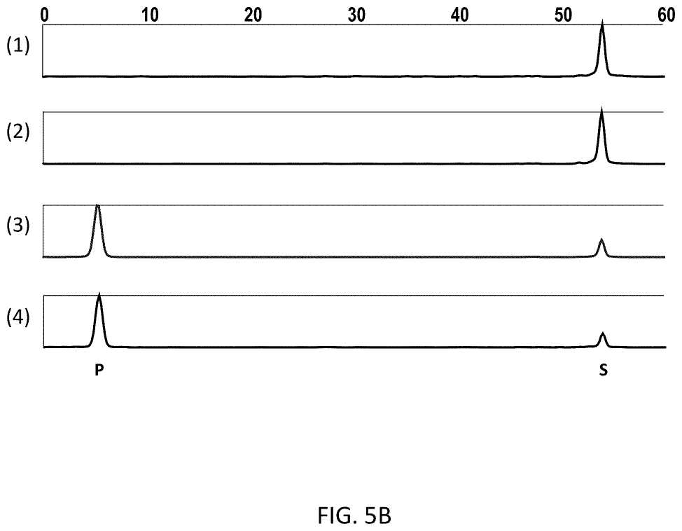

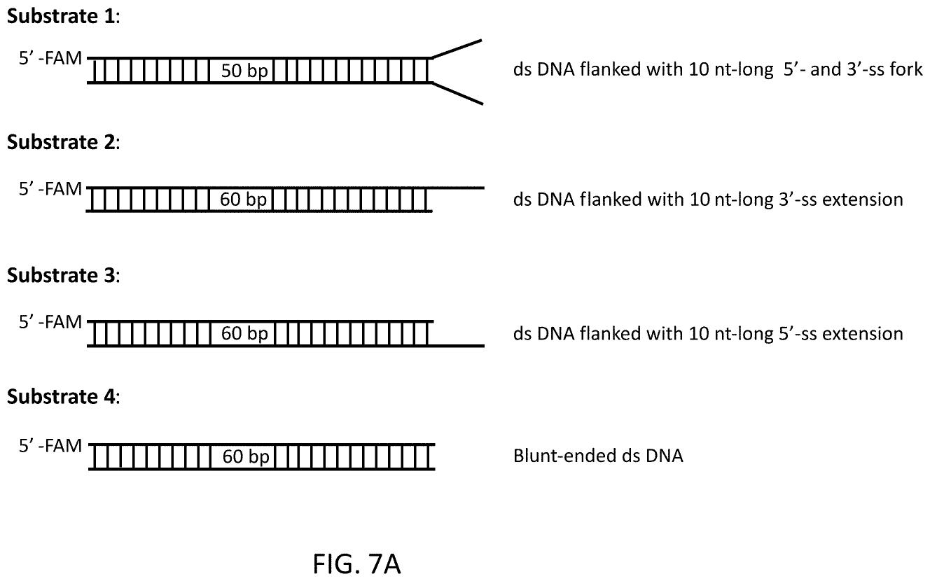

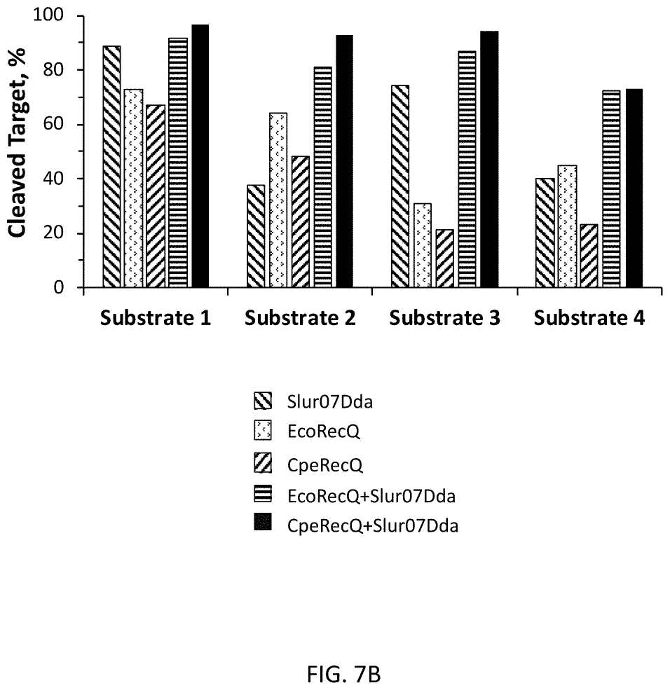

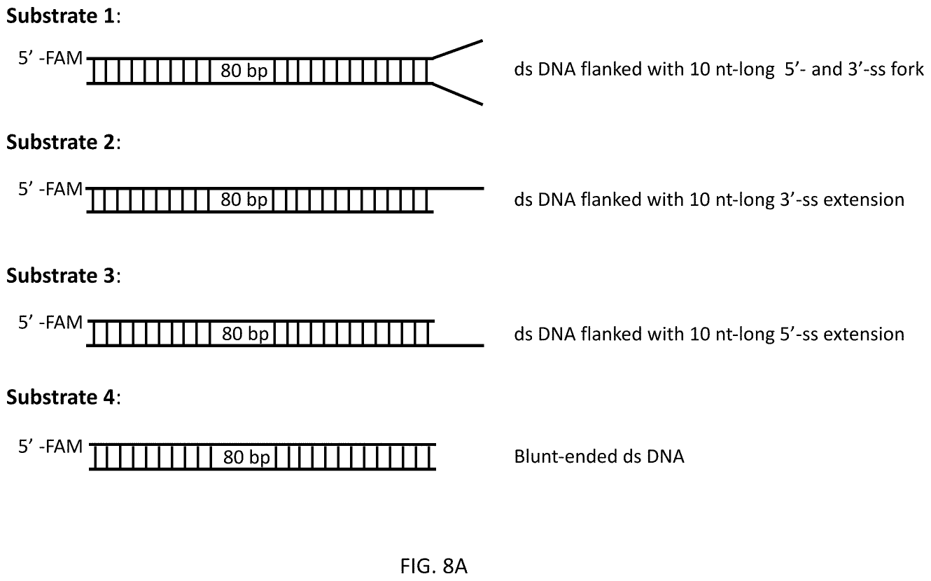

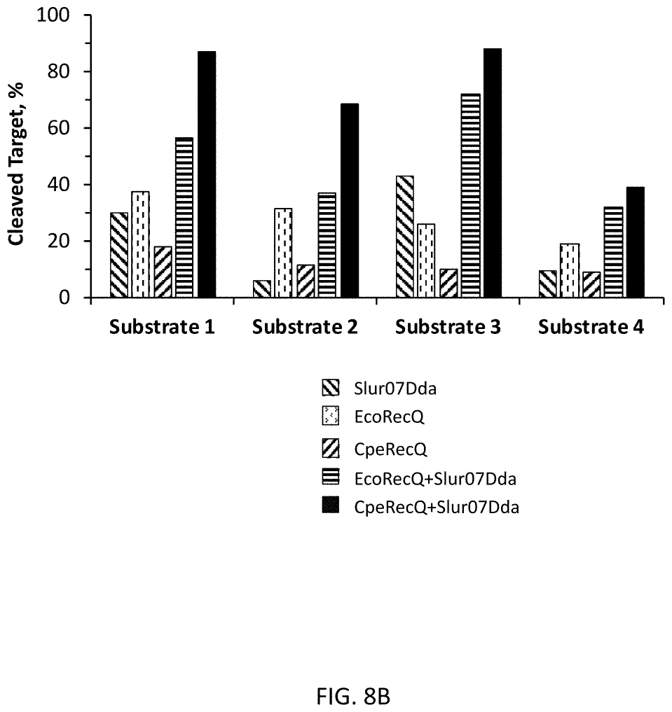

Recently, ds DNA cleavage by mesophilic SeAgo was tested during ongoing transcription of the target region, which was expected to transiently melt ds DNA, but the approach had no observed effect on target cleavage by SeAgo under the conditions tested. Taking a different approach, ds DNA cleavage by argonaute CbAgo was explored during the ongoing unwinding of DNA strands by mesophilic DNA helicases. Applicants disclose here that, surprisingly, mesophilic argonautes are capable of cleaving ds DNA at or near physiological temperatures (e.g., from 25° C. to 45° C.) in the presence of one or more helicases. Initially, CbAgo was combined with either EcoRecQ (Abcam, Inc., Cambridge, Mass., USA) or Slur07 Dda (McLab, South San Francisco, Calif., USA) and both combinations were found to be proficient in cleaving short ds DNA tailed with a single-stranded fork structure (FIG. 5). Most DNA helicases require either a 5′-ss or a 3′-ss DNA end as an initiation point to start unwinding DNA duplex and commonly, but not exclusively, do not bind to blunt-ended ds DNA. At high protein to DNA concentration RecQ-class helicases can initiate duplex DNA unwinding from a blunt end. CbAgo cleavage activity was compared in combination with either Slur07 Dda helicase or two RecQ helicases, EcoRecQ and CpeRecQ, using various either partially or fully paired DNA substrates (FIGS. 7A and 8A). CbAgo displayed cleavage activity on all substrates after reaction was supplemented with any investigated DNA helicase (FIGS. 7B and 8B). RecQ helicases exhibit 3′-5′ unwinding polarity and CbAgo/EoRecQ and CbAgo/CpeRecQ combinations were more active on DNA duplexes carrying 3′-ss tails. In contrast, Slur07 Dda is a 5′→3′ DNA helicase and the CbAgo/Slur07Dda combination was more active on DNA duplexes with 5′-ss tails. All three CbAgo/helicase combinations exhibited a significant activity on blunt-ended DNA indicating that either Slur07 Dda or RecQ were capable of unwinding DNA duplex without single-stranded tails. However, under conditions tested CbAgo activity noticeably declined when duplex portion of each DNA substrate was increased from 60 bp to 80 bp (FIG. 8). To enhance DNA unwinding efficiency, two helicases possessing different unwinding polarity were used concurrently in the CbAgo cleavage reaction. CbAgo cleavage of 60 bp blunt-ended duplex increased by ˜2-3 fold when a helicase mixture consisting of either EcoRecQ+Slur07Dda or CpeRecQ+Slur07Dda was used for strand unwinding (FIGS. 7B and 8B). But again, CbAgo cleavage declined from 70% to 40% when the length of blunt-ended duplex was extended to 80 bp (FIG. 8B). In summary, these experiments demonstrate that CbAgo, in the presence of one or more DNA helicases, may cleave ds DNA at physiological temperature.

Highly processive and highly efficient DNA helicases were evaluated for compatibility with CbAgo. For example, E. coli RecBCD is a very fast (e.g., 1,000 to 2,000 bp s⁻¹) and very processive (e.g., ˜30,000 bp) DNA helicase which prefers unwinding blunt-ended DNA. Wild type RecBCD is a heterotrimer consisting of three subunits, RecB, RecC and RecD and has multiple enzymatic activities: ATP-dependent DNA unwinding activity, ATP-dependent dsDNA and ssDNA exonuclease activity, ATP-stimulated ssDNA exonuclease activity and ATPase activity. Wild type RecBCD possess a strong nuclease activity associated with the RecB subunit. RecB is organized into a 100-kDa N-terminal helicase domain and 30-kDa C-terminal exonuclease/endonuclease domain. The helicase and nuclease domains can function independently from each other suggesting that nucleolytic activity of RecB subunit can be eliminated without losing DNA unwinding function. In one embodiment, a truncated RecB helicase variant, lacking C-terminal 930-1180 amino acids, was constructed and referred to as RecΔB. In another embodiment, a full-length nuclease deficient RecB helicase variant, referred to as RecB^{exo-}, was constructed. Both RecB variants were combined with the RecC subunit to form rapid and processive DNA helicases, referred to as RecΔBC and RecB^{exo-}C.

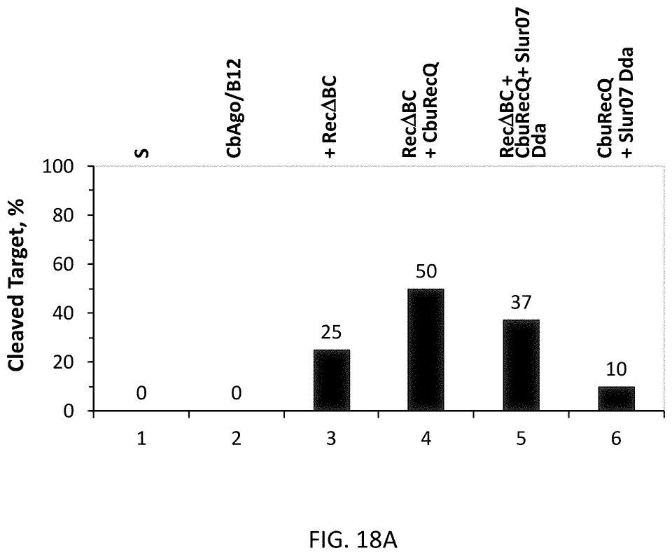

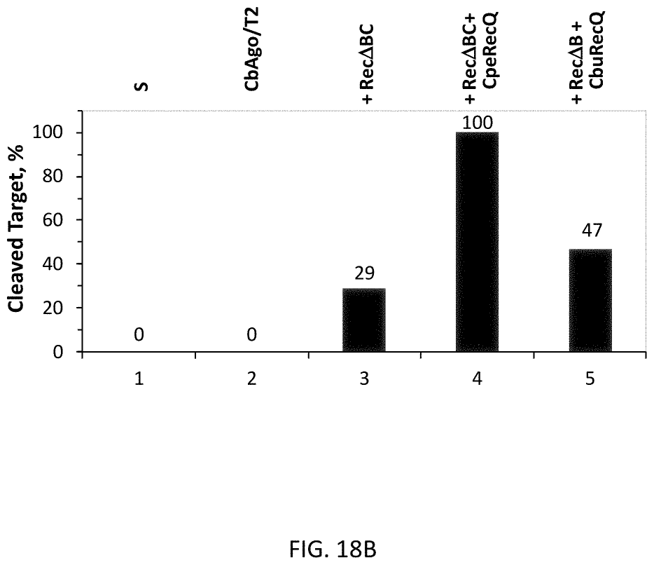

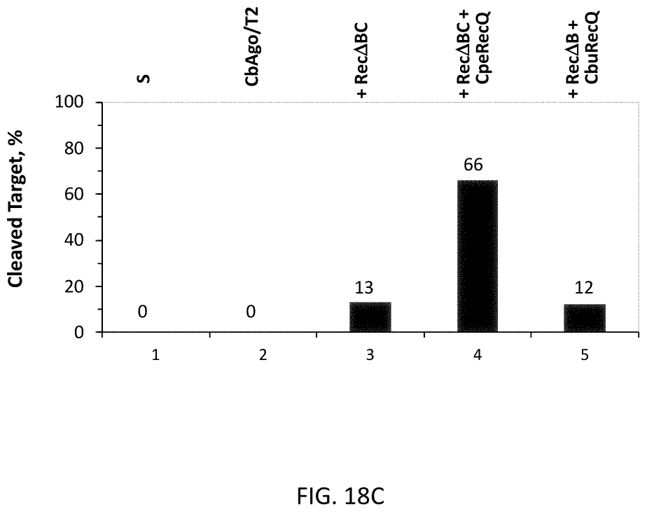

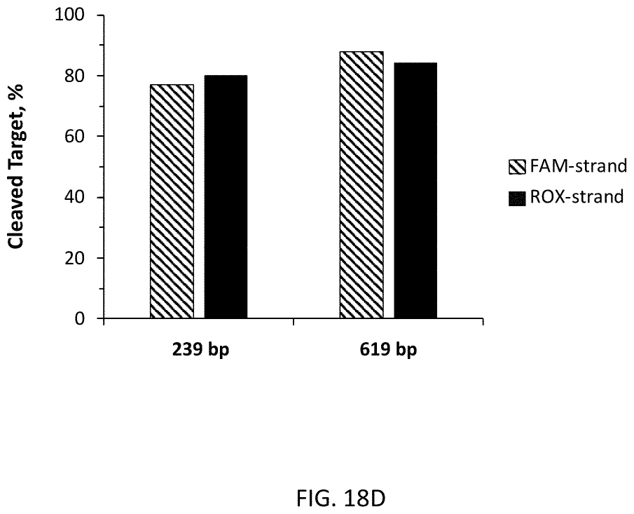

In the presence of RecΔBC DNA helicase CbAgo was capable of cleaving up to 25-30% of targets on blunt-ended DNA (FIG. 18). However, when strand unwinding was carried out concurrently with RecΔBC and CpeRecQ helicases CbAgo cleavage of 239-619 bp blunt-ended DNA increased up to 80% (FIG. 18D). Without limiting any embodiment to a specific mechanism of action, more rapid and processive DNA unwinding activity occurs when RecB subunit forms a complex with RecC subunit. Deletion of the 30 kD C-terminal nuclease domain may have destabilized interactions between the RecΔB and RecC subunits resulting in catalytically unstable RecΔBC complex. The RecD subunit may be used to form a stable RecΔBCD complex and thus, to increase DNA unwinding activity to levels that allow efficient cleavage of long ds DNAs by CbAgo. Likewise, a truncated RecB variant lacking nuclease activity but capable of forming a stable complex with RecC subunit can be created and used in combination with CbAgo for efficient cleavage of long ds DNA at physiological temperatures.

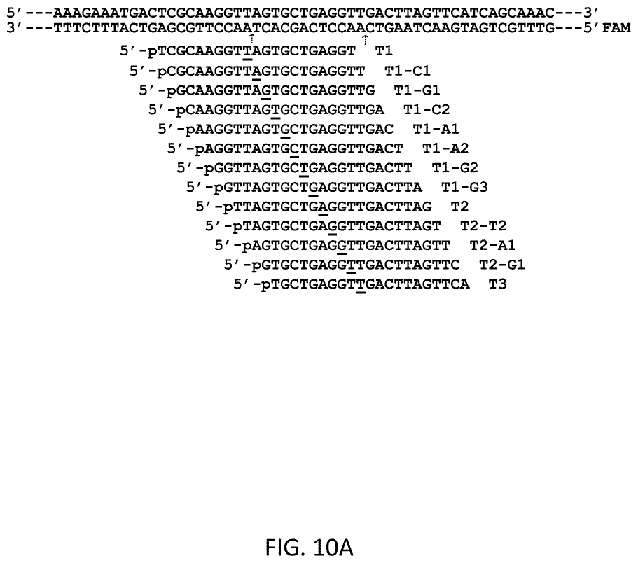

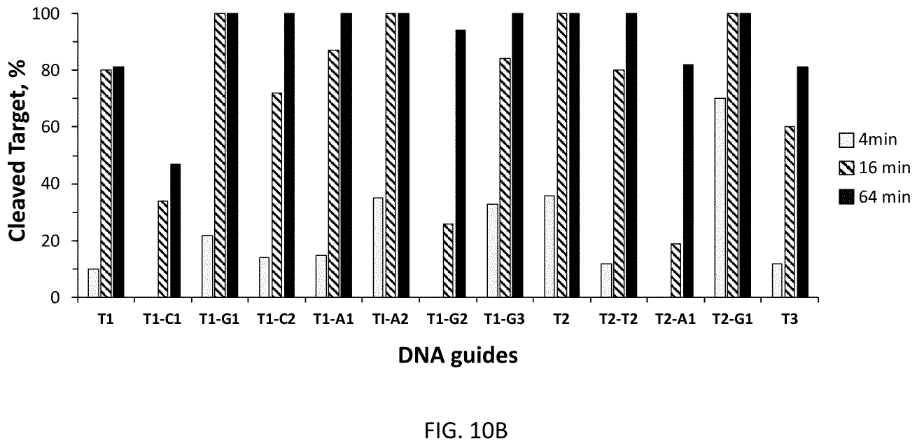

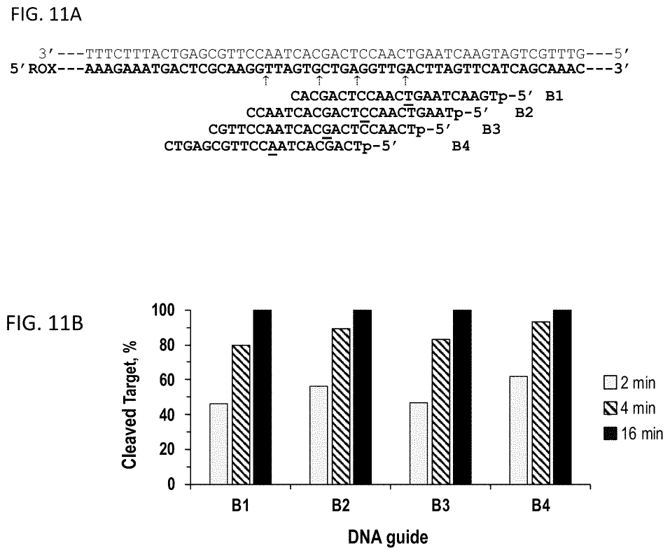

In contrast to RecΔBC, DNA unwinding by RecB^{exo-}C helicase permits CbAgo to efficiently access single-stranded targets on otherwise inaccessible double-stranded substrates. Coupling CbAgo cleavage with DNA strand unwinding by RecB^{exo-}C DNA helicase for the first time allowed argonaute's properties on double-stranded substrates to be evaluated at 37° C. temperature. A detailed analysis of CbAgo cleavage products was carried out using high-throughput capillary gel electrophoreses to identify the most effectual pre-arrangement of the guides for ds target cleavage. An array of 21-nt long DNA guides was used to evaluate cleavage efficiency of individual strands within a double-stranded DNA. In the presence of RecB^{exo-}C helicase, CbAgo loaded with 13 different guides was shown to efficiently cleave targeted DNA strand up to 80-100% in 16 minutes at 37° C. (FIG. 10). Opposite DNA strand targeted with CbAgo loaded with four guides was cleaved even faster as 45-60% of the respective targets were cleaved in the first 2 minutes at 37° C., and a complete target cleavage was achieved after 16-minute incubation (FIG. 11). Altogether, the results presented in EXAMPLE 16 demonstrate that CbAgo can rapidly find and efficiently cleave targets imbedded on long ds substrates during ongoing DNA strand unwinding by RecB^{exo-}C helicase.

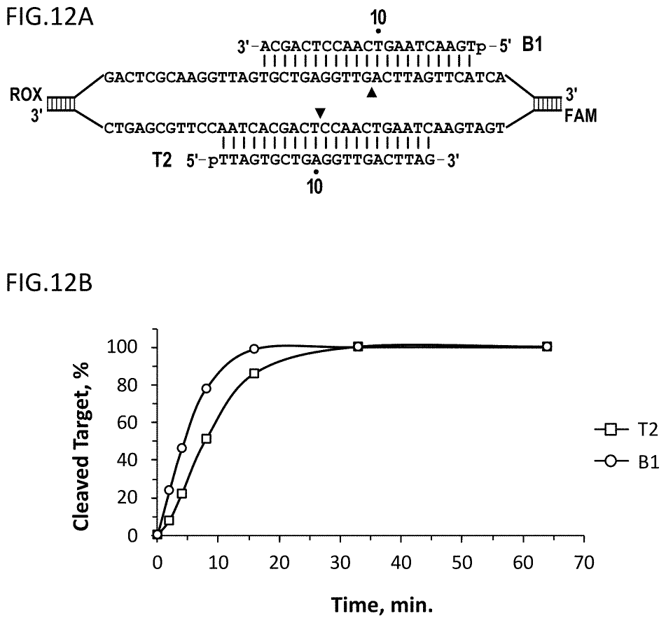

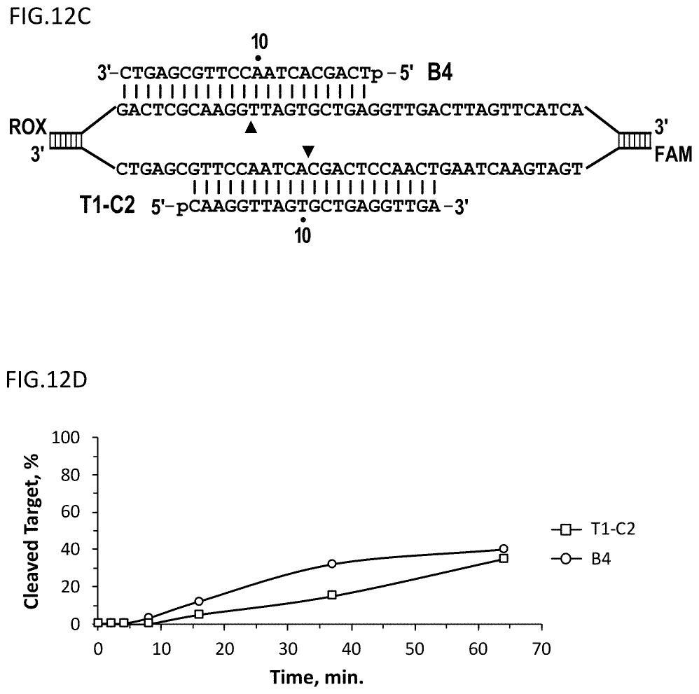

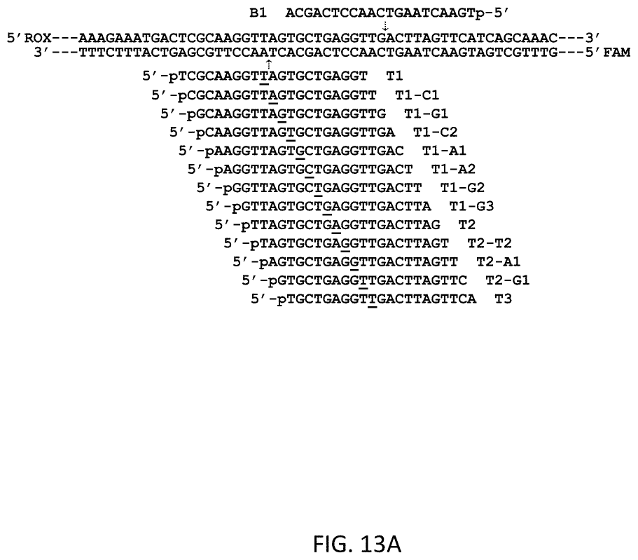

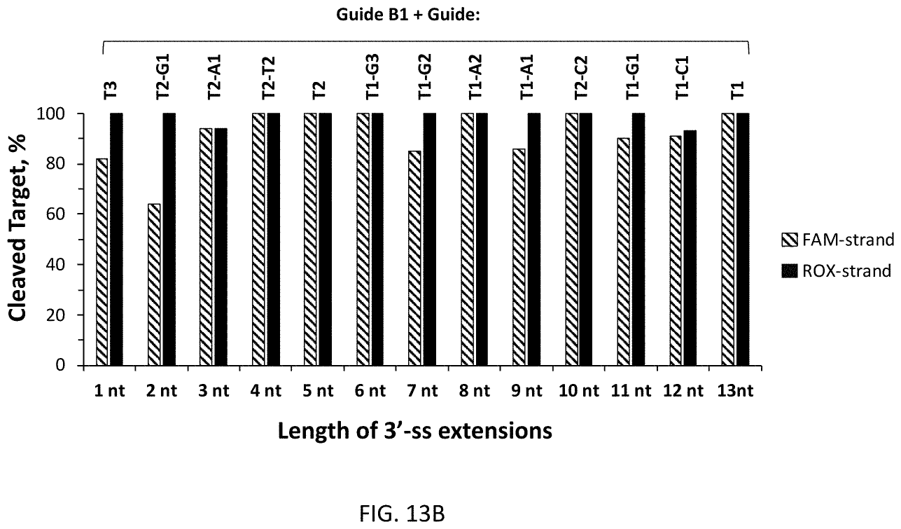

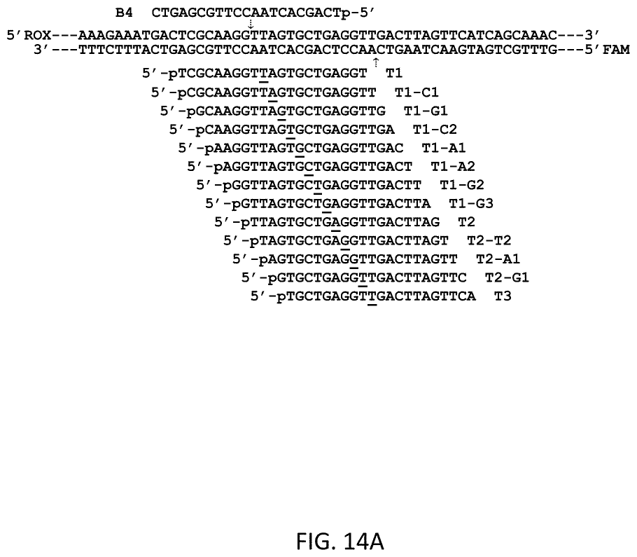

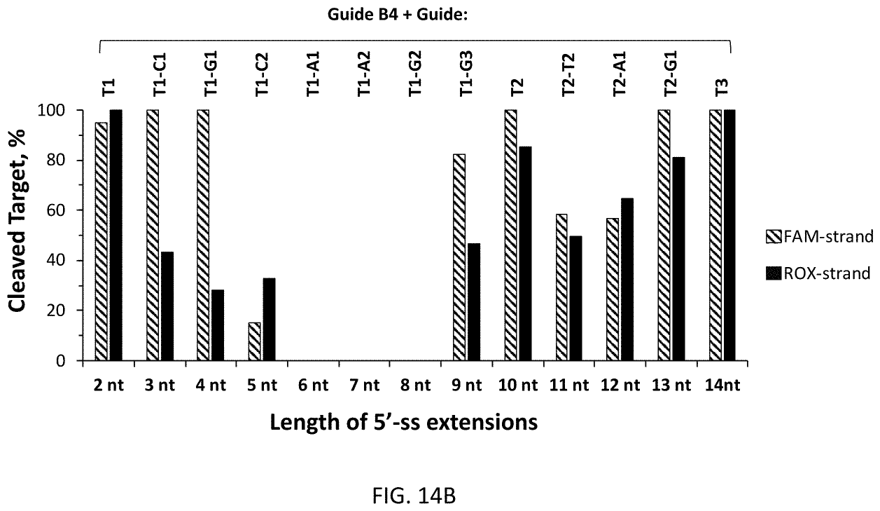

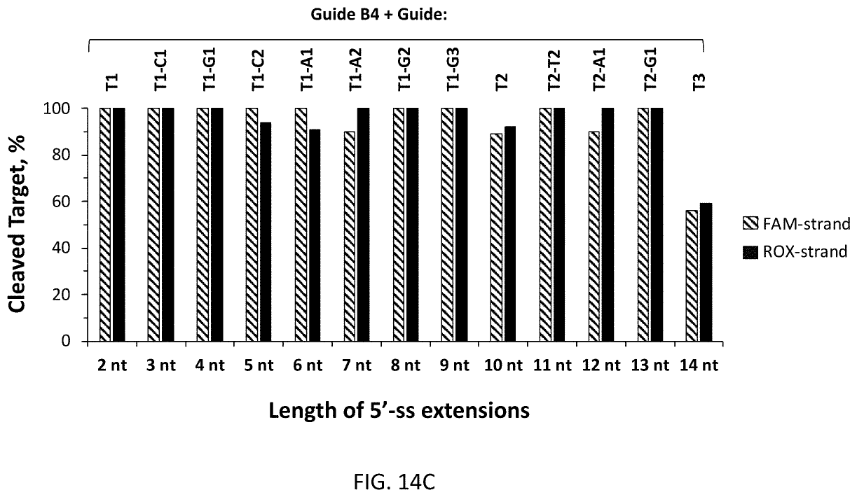

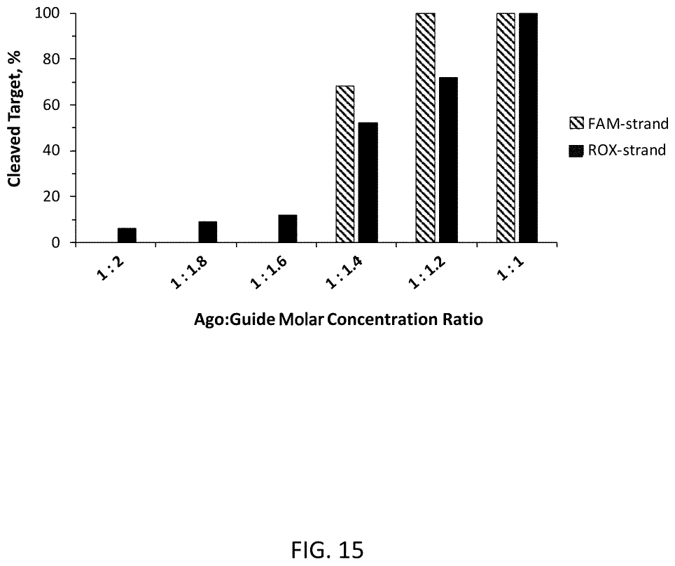

According to some embodiments, individual DNA strands are cleaved independently by two Ago/guide complexes. Cleavage products may be custom-designed (including fragment length, cleavage location, and overhang length, and overhang character (e.g., 5′- or 3′-single-stranded extensions)) by appropriate selection of the guide used to target each DNA strand. In some embodiments, cleavage products may be (further) designed or controlled by adjusting reaction conditions including, for example, molar ratio of Argonaute to guide, molar ratio of Argonaute to substrate, molar ratio of helicase to substrate, reaction temperature, reaction time, salt(s) present (if any) and concentration of such salt(s), pH, buffer(s) present (if any) and concentration of such buffer(s), and combinations thereof. For concurrent cleavage of opposite DNA strands, two guide pairs were initially tested. CbAgo loaded with one guide pair was expected to yield DNA fragments tailed with 5 nt-long 3′-ss extensions (FIG. 12A), whereas CbAgo loaded with a second guide pair was expected to yield DNA fragments tailed with 5 nt-long 5′-ss extensions (FIG. 12C). DNA cleavage by CbAgo loaded with a guide pair that yielded 3′-ss staggered cut advanced to completion and displayed cleavage rates similar to the rates observed in single-guided reactions (FIG. 12B). In contrast, when guides were prearranged to yield a 5′-ss staggered cut, the CbAgo cleavage of both strands declined by ˜10-fold if compared to the respective single-guided reactions suggesting that present arrangement of opposite guides might have an inhibitory effect on ds DNA cleavage (FIG. 12D). The observed differences in CbAgo cleavage efficiency were further investigated using two sets of 13 guide pairs which permitted CbAgo to generate cleavage products flanked with either 3′- or 5′-ss extensions of varying length. Detailed analysis of CbAgo cleavage over time revealed that CbAgo efficiently cleaved both DNA strands when guides were prearranged to yield ds fragments flanked with 3′-ss extensions varying from 1-nt up to 13-nt in length (FIG. 13B). In contrast, CbAgo cleavage efficiency was significantly lower with guides designed to produce ds fragments flanked with 5′-ss extensions (FIG. 14B). Under reaction conditions tested, the CbAgo:guide complexes were formed using a 1:2 molar concentration ratio with intent to abolish a non-specific DNA “chopping” by a guide-free CbAgo. However, at the 1:2 molar concentration ratio tested, free guides are present in a guide loading reaction and the surplus of free guides is then transferred to CbAgo cleavage reaction possibly impacting target cleavage. Additional experiments were conducted using guide pairs which yielded ds fragments flanked with 5′-ss extensions, but CbAgo:guide complexes were formed at a 1:1 molar concentration ratio. CbAgo cleavage efficiency significantly increased when CbAgo was loaded with guides at the 1:1 molar concentration ratio (FIG. 14C). Reactions with varying CbAgo:guide molar concentration ratios performed using a T1-A1+B4 guide pair showed that CbAgo activity significantly increased when CbAgo:guide molar concentration ratio was equal or lower than 1:1.4, and the highest activity was observed at the 1:1 CbAgo:guide molar concentration ratio (FIG. 15).

In some embodiments, guides that target opposite DNA strands may partially complement each other. Complimentary regions may be identified by aligning nucleotide sequences of two guides which are employed concurrently for double-stranded cleavage. For example, the two 21-nt long guides complement each other by 20 nucleotides if they are arranged to yield a blunt-ended ds cut. In case of either 5′- or 3′-staggered cuts which create ss overhangs of 13 nucleotides in length, the 21-nt long guides complement each other by 9 or 11 nucleotides respectively. Sequence alignment also reveals that guides complement each other through 3′-terminal sequences if they are pre-arranged to yield cleavage products tailed with 3′-ss extensions. To generate cleavage products tailed with 5′-ss extensions, the guides must complement each other through their 5′-terminal sequences. FIGS. 12-13 show that regardless of the presence of free guides, CbAgo cleavage may be very efficient if guides complement each other throughout their 3′-ends. But ds DNA cleavage may decline significantly if cleavage reaction carries an excess of free guides that have complementary sequences on their 5′-ends (FIGS. 14-15).

The 5′-end of the guide may play a key role in target recognition and cleavage by Ago nuclease. Nucleotides 2-8 of the guide counted from a 5′-phosphorylated end are termed the “seed” region. In an Ago/guide complex, the bases of the seed region are solvent exposed, therefore they can readily base pair with a matching sequence on the target strand. If targeted sequence matches the guide sequence over more than 15 nucleotides, then Ago readily cleaves targeted sequence at the scissile phosphate which is located between nucleotides 10 and 11 counting from the 5′-phosphate on the guide sequence. When two opposing CbAgo:guide complexes are formed at a 1:2 molar concentration ratio and then combined in the same cleavage reaction, free guides may hybridize to each other through complementary regions and form inactive double-stranded structures. However, in some embodiments, the free guides may also base pair with the seed region of the already formed CbAgo/guide complex, thus, turning into unintentional targets. Without limiting any embodiment to any particular mechanism of action, CbAgo may cleave such unintentional target, for example, where CbAgo/guide complex is capable of base pairing with the free guide by more than 15 nucleotides. Close inspection of selected CbAgo reactions performed with guides sharing lengthy complementary regions revealed slow cleavage of ds targets during early time points followed by a rapid acceleration in cleavage at the later time points, thus, indicating that single-stranded free guides possibly were cleaved prior to double-stranded DNA targets. In some embodiments, CbAgo cleavage of ds DNA may be inhibited, possibly severely inhibited, in the presence of free guides which are highly complementary to each other across the 5′-terminal sequences. Without limiting any particular embodiment to any specific mechanism of action, this inhibition may be due to the formation of uncleavable double-guided complexes.

The present disclosure provides methods for effective pre-arrangement of the guides for ds DNA cleavage by CbAgo in the presence of RecB^{exo-}C DNA helicase. For example, efficient methods for double-strand cleavage by CbAgo may include targeting ds DNA with guide pairs programmed to yield 3′-ss staggered breaks. Efficient methods for double-strand cleavage by CbAgo may include targeting ds DNA with guide pairs programmed to produce 5′-ss staggered breaks, wherein the CbAgo:guide molar concentration ratio is equal to or lower than 1:1.4.

CbAgo loaded with several particular guides (e.g., guides T1-C1 or T2-A1) cleaved single-stranded target imbedded within a double-stranded DNA (FIG. 10B) less effectively than other guides. However, the ds cleavage efficacy with these guides improved when combined with a guide (e.g., B1) which was effective in targeting complementary strand (FIG. 10B and FIG. 13B). The apparent stimulation of one CbAgo/guide complex by the opposite CbAgo/guide complex suggests a possible incidental cooperation between the two complexes. Without limiting any embodiment to any particular mechanism of action, the fast-acting CbAgo/guide complex (e.g., CbAgo/B1) may remain bound to DNA after rapid target cleavage, thus slowing down re-annealing of complementary DNA strands. As a result, DNA strands stay separated long enough for cleavage of the opposite DNA strand by an otherwise less effective CbAgo/guide complex (e.g., CbAgo/T1-C1 or CbAgo/T2-A1). The present disclosure suggests that to achieve an efficient double-strand cleavage a selected guide pair should consist of at least one guide which rapidly cleaves the targeted strand.

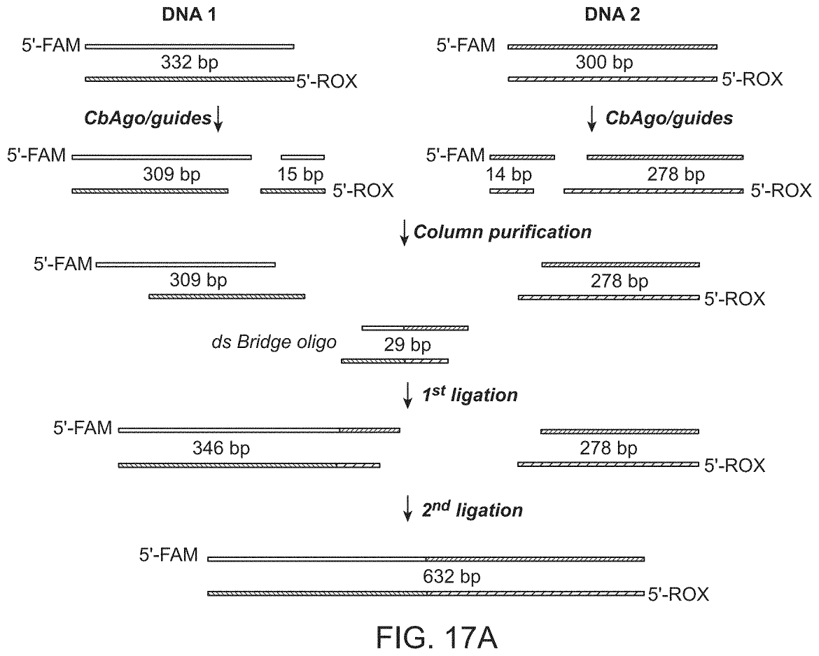

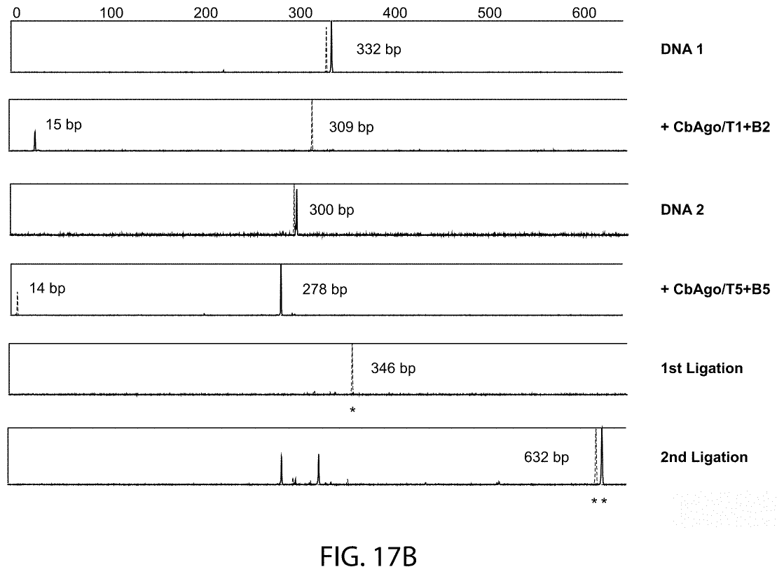

The present disclosure provides mesophilic Ago which can rapidly cleave ds targets during concurrent DNA unwinding by DNA helicase RecB^{exo-}C deficient in nuclease activity. As elaborated in EXAMPLES 17-18, in the presence of RecB^{exo-}C helicase CbAgo efficiently cleaves linear ds DNAs ranging from 300 bp up to 25 kb in length. According to some embodiments, CbAgo/RecB^{exo-}C combination cleaves ds targets located as far as 11-12 kb away from the end of linear DNA. But also, the CbAgo/RecB^{exo-}C can be used to cleave close to the end of linear DNA, thus yielding DNA fragments with sequence-specific ss extensions of any desirable length that are ready for ligation without further enzymatic treatment. EXAMPLE 19 discloses methods for assembly of natural linear DNA molecules by using CbAgo/RecB^{exo-}C programmable DNA endonuclease (FIG. 17).

According to some embodiments, an argonaute and a helicase, for example, CbAgo and RecB^{exo-}C, may be combined for efficient mesophilic DNA-guided DNA-cleaving programmable endonuclease activity. These may be used in vitro for development of new synthetic biology tools that require or benefit from sequence-specific nicking/cleavage of double-stranded DNA at otherwise inaccessible locations.

### General Considerations

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation. As used herein, “can” and “may” are intended to convey an optional, possible, and/or permissive condition of or for operability.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “Argonaute” refers to an endonuclease that catalyzes cleavage of a single stranded nucleic acid governed by the sequence of a bound guide and may comprise an N-terminal domain (e.g., facilitating release of a target nucleic acid after cleavage), a PAZ domain (e.g., which may hold the 3′ end of the guide pending hybridization of the guide with a complimentary sequence), a MID domain (e.g., which binds a short, single-stranded oligonucleotide guide), and/or a PIWI domain (e.g., having a metal-dependent, RNase H-like endonuclease with activity conditioned on whether the PAZ domain is bound to the 3′ end of the guide and/or whether the guide is hybridized to a complementary target sequence). In some embodiments, an Argonaute may be a naturally occurring protein. In some embodiments, an Argonaute may be a non-naturally occurring protein. An Argonaute may have an amino acid sequence having at least 80%, at least 90%, at least 95%, or 100% sequence identity to a wild type Argonaute polypeptide (e.g., Argonaute from Thermus thermophilus). Examples of Argonautes include, without limitation, Argonautes from Aquifex aeolicus, Microsystis aeruginosa, Clostridium bartlettii, Exiguobacterium, Anoxybacillus flavithermus, Halogeometricum borinquense, Halorubrum lacusprofundi, Aromatoleum aromaticum, Thermus thermophilus, Synechococcus, (e.g., Synechococcus elongatus), Thermosynechococcus elogatus, C. butyricum (CbAgo), C. disporicum (CdAgo), C. perfringens (CpAgo), C. sartagoforme (CsAgo), C. saudiense (CaAgo), I. bartlettii (IbAgo), and/or an Argonaute listed in Table 2 or Table 3.

Argonautes include eukaryotic (e.g., mouse AGO2) and prokaryotic Argonautes. Argonaute may comprise an amino acid change relative to a reference sequence (e.g., a naturally occurring sequence) such as a deletion, insertion, substitution, variant, mutation, fusion, chimera, or any combination thereof. This term refers to any modified (e.g., shortened, mutated, lengthened) polypeptide sequence or homologue of the Argonaute. An Argonaute can be enzymatically inactive, partially active, constitutively active, fully active, inducibly active and/or more active, (e.g., more than the wild type homologue of the protein or polypeptide). A “thermostable” Argonaute is a protein that remains catalytically active for at least 5 minutes or 10 minutes at elevated temperatures such as above 45° C., 50° C. or 55° C. An Argonaute catalytically active at physiological temperatures (e.g., 25-45° C.) may be referred to as a “mesophilic Argonaute”. With its guide bound to a complementary target sequence, an Argonaute creates a break in the phosphodiester backbone of the complementary target nucleic acid. In the case of double-stranded substrates, a break is only created in the strand which is complementary to the guide nucleic acid. As disclosed herein, a break in the other strand may be introduced using a second Argonaute with a second guide.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include, for example, Tris, HEPES, TAPS, MOPS, tricine, or MES.

In the context of the present disclosure, “double-strand break” refers to any breakage of the phosphate backbones of each strand in a double stranded polynucleotide (e.g., RNA, DNA, RNA/DNA hybrids). Double stranded polynucleotides may have any helical (e.g., α, β, ζ) or non-helical conformation. Double strand breaks include breaks that leave blunt ends (Class I) or overhangs (Classes II and III). Class II overhangs may be from 1-50 nucleotides in length (e.g., 1-4 nts, 5-12 nts, 13-25 nts, 26-50 nts) with the stringency of conditions needed for separation of such overhangs generally increasing with length of the overhang. Overhangs of 1-4 nts may be generated by restriction endonucleases. Overhangs of 5-12 nts may be dissociated, for example, at 37 C. Overhangs of 13 nts or beyond may require higher temperatures and/or chemical denaturants. Class III overhangs may be over 50 nucleotides in length. A double-strand break in a double-stranded polynucleotide may produce two fragments of the original double-stranded polynucleotide. A double strand break may not result in two separate fragments, for example, if an overhang is long and/or conditions permit the overlapping regions of each strand to remain base paired. In such cases, the overhang may be destabilized (e.g., by heat, salt, and/or pH adjustments) to release the overlapping strands from one another.

In the context of the present disclosure, “guide” refers to a single-stranded oligonucleotide (a) capable of binding (e.g., hybridizing to) a polynucleotide having a complimentary sequence, (b) capable of binding an Argonaute, and (c) comprising (i) at least 12 nucleotides (e.g., 12-60 nucleotides), (ii) at least 50% deoxyribonucleotides (e.g., at least 60%, at least 70%, at least 80%, at least 90%, or 100% deoxyribonucleotides), (iii) up to 50% ribonucleotides (e.g., up to 10%, up to 25%, up to 35%, up to 45%, up to 50% ribonucleotides), (iv) optionally, a phosphorylated 5′ end, (v) optionally, a nucleotide sugar modification, and (vi) optionally, a nucleotide substitution. In some embodiments, a guide may comprise a phosphorylated 5′ end or another chemical modification at its 5′ end. A guide may be engineered or synthetic with a sequence selected to complement a desired target sequence. A guide maybe capable of directing an Argonaute polypeptide:guide DNA complex to a target polynucleotide. A DNA guide may be an oligonucleotide or polynucleotide that is synthetic or from a natural source such as genomic DNA, cDNA, extrachromosomal DNA, microbial DNA or viral DNA (e.g., the natural source differing from the Argonaute such that the guide and Argonaute together form a non-naturally occurring combination). The guide DNA is generally single stranded when used with Argonaute although it may be derived from dsDNA.

In some embodiments, a guide length suitable for Argonaute cleavage of dsDNA (e.g., in the presence of a helicase or single strand binding protein) may comprise at least 12 nucleotides, for example, having a size range of 12-60 nucleotides, 14-50 nucleotides, 15-40 nucleotides, 16-35 nucleotides, 15-24 nucleotides, or 16-21 nucleotides. In some embodiments, a guide DNA may be greater than 21 nucleotides or at least 24 nucleotides in length. In some embodiments, a guide may be 16-21 nucleotides in length (e.g., 16, 17, 18, 19, 20 or 21 nucleotides).

In some embodiments, a guide may comprise a nucleotide sugar modification or a nucleotide substitution. In some embodiments, a nucleotide sugar modification comprises a 2′ sugar modification and maybe selected from the group consisting of a 2′-O—CH₃, a 2′-F, and a 2′-MOE modification. In some embodiments, a nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In some embodiments, a guide molecule comprises a sugar modification and a nucleotide substitution.

The nucleotide sequence of a guide may or may not be degenerate. For example, guides with degenerate sequences may be useful for targeting nucleic acid sequences that are not fully known and/or for targeting more than one variant in a population of polynucleotides.

In the context of the present disclosure, “helicase” refers to a motor protein that moves linearly along double stranded nucleic acids unwinding or otherwise separating the component strands along base paired nucleosides. A helicase may or may not form a ring structure surrounding a nucleic acid substrate. A helicase may or may not unwind molecules that comprise partially single stranded nucleic acids (“a helicases”). Examples of helicases include, without limitation, RecQ-family helicases (e.g., EcoRecQ DNA helicase from Escherichia coli (WP_096324295.1), CpeRecQ from Clostridiumperfringens (WP_011590145.1), Cbu RecQ from Clostridium butyricum (WP_003411240.1)); DNA helicases from T4-like bacteriophages (e.g., T4 gp41, T4 gp41 associated with T4 gp59, T4 UvsW, T4 Dda and Slur07 Dda); T7 bacteriophage gp4 DNA helicase; RecBCD-family helicases (e.g., E. coli RecBCD DNA helicase); modified RecBCD helicases (e.g., RecB^{exo-} helicase, RecB^{exo-} C, RecB^{exo-}CD, RecΔB, RecΔBC, RecΔBCD); UvrD/PcrA family helicases, e.g., E. coli EcoUvrD, E. coli Rep, M. tuberculosis PcrA, M. leprae PcrA; and/or E. coli Tra helicase. A helicase may unwind, for example, linear, nicked circular, and/or supercoiled circular DNA.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., ATP, TTP, CTP, GTP, a buffer, a detergent, a dye, a solvent or a preservative).

In the context of the present disclosure, “oligonucleotide” refers to a polymer of nucleotides comprising naturally occurring nucleotides, non-naturally occurring nucleotides, derivatized nucleotides, or a combination thereof. As used herein, the term “complementarity” refers to the ability of nucleotides, or analogues thereof, to form Watson-Crick base pairs. Complementary nucleotide sequences will form Watson-Crick base pairs and non-complementary nucleotide sequences will not.

In the context of the present disclosure, “single-stranded DNA binding protein” refers to a protein that binds to ssDNA. The genomes of most organisms, including bacteria (e.g., E. coli), viruses (e.g., herpes viruses) and mammals, encode at least one SSB. SSBs of interest include, but are not limited to, ET SSB, E. coli recA, T7 gene 2.5 product (gp2.5), T4 gene 32 product (gp32), E. coli SSB, replication protein A (RPA) from archaeal and eukaryotic organisms, Nanoarchaeum equitans SSB-like protein, UvrD, RadA, Rad51, phage lambda RedB or Rac prophage RecT. An SSB may be thermostable or mesolabile. An SSB may have at least 80%, at least 90%, at least 95%, or 100% sequence identity to a wild type SSB.

In the context of the present disclosure, a “substitution” at a position in a comparator amino acid sequence refers to any difference at that position relative to the corresponding position in a reference sequence, including a deletion, an insertion, and a different amino acid, where the comparator and reference sequences are at least 80% identical to each other. A substitution in a comparator sequence, in addition to being different than the reference sequence, may differ from all corresponding positions in naturally occurring sequences that are at least 80% identical to the comparator sequence.

In the context of the present disclosure, “target” refers to a nucleic acid having a nucleic acid sequence, which may be a chromosomal sequence or an extrachromosomal sequence, (e.g., an episomal sequence, a minicircle sequence, a plasmid, a mitochondrial sequence, a chloroplast sequence, etc.). A target nucleic acid can be a dsDNA or ssDNA; a target nucleic acid may also be an RNA.

All publications, patents, and patent applications mentioned in this specification are herein incorporated by reference to the same extent as if each individual publication, patent, or patent application was specifically and individually indicated to be incorporated by reference. All reagents referenced, if unavailable elsewhere, may be obtained from the indicated source and/or New England Biolabs, Inc. (Ipswich, Mass.).

### Compositions

The present disclosure provides, in some embodiments, compositions for producing a break in double-stranded DNA (e.g., linear DNA, open circular DNA, negatively supercoiled DNA, and/or positively supercoiled DNA). In some embodiments, compositions may produce a break in double-stranded DNA at temperatures from, for example, 25° C. to 45° C. (e.g., 25° C. to 30° C., 30° C. to 35° C., 35° C. to 40° C., or 40° C. to 45° C.). A composition may comprise, for example, a helicase, an Argonaute bound to a first guide, and optionally, an Argonaute bound to a second guide. In some embodiments, a single Argonaute with a single guide may produce a double-stranded break. For example, if a palindromic ds sequence (e.g., 5′-CGTAATTCGTACGAATTACG-3′/3′-GCATTAAGCATGCTTAATGC-5′; SEQ ID NO:94/SEQ ID NO:94) is targeted, an Argonaute having a guide 5′-pCGTAATTCGTACGAATTACG-3′ (SEQ ID NO:94) (a) may bind a target's top strand resulting in cleavage of the top strand and (b) may bind complementary bottom strand resulting in cleavage of the bottom strand, thereby producing a double-stranded break. Double-stranded DNA with longer and/or more complex repeating sequences may also be cleaved with an Argonaute bound to a single guide sequence.

Compositions for cleaving complex and/or non-palindromic/non-repeating target sequences may comprise, a helicase, an Argonaute bound to a first guide, and an Argonaute bound to a second guide. The first and second guides may be selected to target sequences on opposite strands such that the resulting single-stranded breaks are on opposite strands and close enough to one another to constitute a double-strand break. In some embodiments, a composition may include or exclude a single-stranded DNA binding protein.

A composition, according to some embodiments, may include or exclude components beyond a helicase and an Argonaute. For example, a composition may include one or more polynucleotides that are actual or potential substrates for programmed cleavage (e.g., plasmids, phage, vectors, genomic DNA, organellar DNA, library DNA), detection agents, nucleotide triphosphates (e.g., ATP, GTP, CTP, TTP or modified versions thereof), buffers, salts, detergents, and/or crowding agents. A composition may include one or more other proteins, examples of which include polymerases, ligases, nucleases, helicase loading proteins (e.g., T4 gp59 protein for T4 gp41 helicase or MutL protein for EcoUvrD helicase), and/or helicase processivity enhancers (e.g., RepD for PcrA helicase).

In some embodiments, a helicase may be in a molar excess relative to Argonaute. In some embodiments, an Argonaute and a helicase may be present in a reaction at molar a ratio of 1 (Argonaute):1 (helicase) to 1(Argonaute):100 (helicase), e.g., 1 (Argonaute):5 (helicase) to 1 (Argonaute):30 (helicase), e.g., about 1 (Argonaute): about 15 (helicase), although ratios outside of these ranges can be used.

In some embodiments, off-target activity may be reduced or absent from compositions and methods of the disclosure. Selection of the length, complexity, and/or G:C content of the guide sequence(s), for example, may result in compositions and methods for cleaving DNA with little or off-target activity.

### Methods

The present disclosure provides, in some embodiments, methods of forming a double strand break in a double-stranded polynucleotide at a (pre-selected) target position in the polynucleotide. According to some embodiments, forming a double strand break in a double-stranded polynucleotide my occur at temperatures from, for example, 25° C. to 45° C. A method may comprise, for example, contacting (a) a double-stranded polynucleotide having a first target sequence on a first strand of the polynucleotide and a second target sequence on the opposite strand, (b) a helicase, (c) an Argonaute with a first bound guide (e.g., having a sequence complimentary to the first target sequence), and (d) an Argonaute (which may be the same as or different from the Argonaute in (c)) with a second bound guide (e.g., having a sequence complimentary to the second target sequence) under conditions that permit hybridization of complimentary sequences and cleavage of the first strand by the (c) Argonaute and cleavage of the second strand by the (d) Argonaute to produce a double strand break in the polynucleotide, the double strand break forming at least a first fragment of the polynucleotide and a second fragment of the polynucleotide. Pre-selecting a position for cleavage may include, in some embodiments, obtaining the nucleotide sequence of at least a portion of each strand of the polynucleotide, selecting for each strand a cleavage site in the obtained sequence, identifying a sequence for a first guide complementary to at least a portion of the obtained sequence for one strand and sufficient to contact an Argonaute bound to such first guide and the cleavage site.

In some embodiments, a method may further comprise detecting at least one of the fragments of the polynucleotide, for example, by gel-electrophoresis, capillary electrophoresis, mass spectrometry, fluorescence (e.g., where a fluorescent tag and/or a quencher is/are attached to the polynucleotide or a fragment), and/or sequencing.

### EXAMPLES

Some specific example embodiments may be illustrated by one or more of the examples provided herein.

### Example 1: pAgo Protein Expression and Purification

T7 Express lysY/I^{q} E. coli (New England Biolabs, Inc., Ipswich, Mass., USA) carrying expression plasmid was grown at 37° C. in 1 L of LB medium containing 0.02 mg/ml kanamycin until OD₆₀₀ 0.6. Protein expression was induced with 0.2 mM IPTG and cell culture continued to grow overnight at 16° C. The cells were harvested by centrifugation, resuspended in 45 ml buffer A (20 mM Tris-HCl, pH 7.5, 300 mM NaCl, 10 mM imidazole) supplemented with protease inhibitor cocktail (Complete mini. EDTA-free; Roche Diagnostics GmbH, Mannheim. Germany) and disrupted by sonication. The cell-free lysate was loaded on a 5 ml HisTrap Nickel HP column (GE-Healthcare, Chicago, Ill., USA) and proteins were eluted with 20-250 mM imidazole gradient. The fractions containing Ago protein were pooled, diluted 2-fold with buffer B (20 mM Tris-HCl, pH 7.5, 0.1 mM EDTA), loaded on 5 ml HiTrap Heparin HP column (GE-Healthcare, Chicago, Ill., USA) and proteins were eluted with 0.15-1 M NaCl gradient. The fractions containing Ago protein were pooled, diluted with buffer B to 150 mM NaCl concentration, loaded on 5 ml HiTrap Capto S Sepharose HP column (GE-Healthcare, Chicago, Ill., USA) and eluted with 0.15-0.5 M NaCl gradient. The fractions containing Ago were pooled and loaded on 5 ml Bioscale CHTP column (Bio-Rad, Hercules, Calif., USA). The proteins were eluted with 0.02-0.3 M KPO4 gradient. The purified protein was concentrated by dialysis against 20 mM Tris-HCl (pH 7.5), 300 mM NaCl, 0.1 mM EDTA, 0.1 mM TCEP and 50% (vol/vol) glycerol and stored at −80° C. The purified Argonaute proteins were >95% homogenous as determined by Coomassie-stained SDS-PAGE.

### Example 2: RecB Protein Purification

T7 Express E. coli (New England Biolabs, Inc., Ipswich, Mass., USA) carrying RecB^{exo-} expression plasmid was grown at 37° C. in until OD₆₀₀=0.6. Protein expression was induced with 0.2 mM IPTG and cell culture continued to grow overnight at 16° C. The cells were harvested by centrifugation, resuspended in 150 ml buffer A (20 mM potassium phosphate, pH 7.0, 300 mM NaCl, 20 mM imidazole. 5% glycerol) and disrupted by sonication. The clarified lysate was loaded on a HisTrap Nickel HP column (5 ml) and proteins were eluted using 20-250 mM imidazole gradient in buffer A. The fractions containing RecB^{exo-} protein were diluted 2-fold with buffer B (20 mM potassium phosphate. pH 7.0, 0.1 mM EDTA, 5% glycerol) to bring NaCl concentration to 100 mM and loaded on HiTrap Heparin HP column (5 ml). The proteins were eluted using 0.1-0.8 M NaCl gradient in buffer B. The fractions containing RecB^{exo-} protein were diluted 2-fold with buffer B and loaded on Bioscale CHTP column (5 ml). The proteins were eluted with 0.02-0.4 M KPO4 gradient in buffer B containing 250 mM NaCl. The purified protein was concentrated by dialysis against 10 mM Tris-HCl, pH 7.4, 300 mM NaCl, 0.1 mM EDTA, 0.1 mMDTT and 50% glycerol. The protein was >95% homogenous as determined by Coomassie-stained SDS-PAGE.

### Example 3: RecC Protein Purification

T7 Express E. coli carrying RecC expression plasmid was grown and expressed as described above for RecB^{exo-} protein. The cells were resuspended in 80 ml of 20 mM potassium phosphate buffer, pH 7.0 containing 450 mM NaCl, 20 mM imidazole and disrupted by sonication. The clarified lysate was loaded on a HisTrap Nickel HP column (5 ml) and proteins were eluted with 20-250 mM imidazole gradient in the buffer that contained 300 mM NaCl. The pooled RecC protein was diluted 3-fold with 20 mM potassium phosphate buffer. pH 7.0, 0.1 mM EDTA, 5% glycerol to bring NaCl concentration to 100 mM and was loaded on HiTrap Heparin HP column (5 ml). RecC protein did not bind to the Heparin column at 100 mM NaCl. The flow through fraction was further diluted to 75 mM NaCl concentration and loaded on HiTrap Q HP column (5 ml). RecC protein was eluted with 0.75-1.0 M NaCl gradient and concentrated by dialysis against 10 mM Tris-HCl, pH 7.4, 300 mM NaCl, 0.1 mM EDTA, 0.1 mM DTT and 50% glycerol. The protein was >90% homogenous as determined by Coomassie-stained SDS-PAGE.

### Example 4: Constructs for pAgo Expression and Purification

The codon optimized genes encoding Argonaute proteins were ordered in pET29a expression vector from GenScript (Piscataway, N.J., USA). Analytical amounts of twenty Argonaute proteins were synthesized from pET29a plasmids using PURExpress In Vitro Protein Synthesis kit (New England Biolabs, Inc., Ipswich, Mass., USA). For large scale expression and purification of CbAgo, CpAgo, CdAgo, IbAgo, CsAgo and CaAgo, the respective genes were subcloned into pET28c expression vector in frame with the N-terminal 6×His tag. Argonaute protein expression and purification procedures are provided in EXAMPLE 1.

### Example 5: Construction of recB^{exo-} and recC Expression Clones

Exonuclease V encoding plasmid construct (New England Biolabs, Inc. Ipswich, Mass.) was used as a template to construct nuclease deficient mutant of RecB DNA helicase. A full-length RecB^{exo-} mutant was created by introducing three mutations within the catalytic site of nuclease domain. The recB coding sequence was amplified as three overlapping PCR fragments B1 (3080 bp). B2 (200 bp) and B3 (340 bp). To introduce E1020A mutation, the GAG codon was replaced by GCG codon in the overlapping primers used for amplification of fragments B1 and B2. Similarly, codon GAC was changed to GCC and codon AAA was changed to GCA in the overlapping primers for amplification of fragments B2 and B3 that resulted in D1080A and K1082A mutations (see TABLE 1 for complete primer sequences). The three recB fragments were directly assembled into pET28c vector in frame with the N-terminal 6×His tag employing NEBuilder HiFi DNA Assembly Cloning Kit (New England Biolabs. Inc., Ipswich. Mass.). The fragment of recB gene encoding for the N-terminal 1-929 amino acids was amplified by PCR and assembled into pET28c vector in frame with the N-terminal 6×HIs tag to create a RecΔB deletion mutant. Wild type RecC encoding gene was individually sub-cloned into pET28c vector in frame with the N-terminal 6×His tag. RecΔB, RecB^{exo-} and RecC protein expression and purification is provided in EXAMPLE 2 and EXAMPLE 3.

### Example 6: Argonaute Cleavage Assays on Single-Stranded DNA or RNA Substrates

All substrate oligonucleotides (DNA or RNA) and 5′-phosphorylated guides were purchased from Integrated DNA technologies (Coralville, Iowa, USA). Nucleotide sequences for all substrates and guides can be found in TABLE 1.

To test activity of in vitro expressed pAgo proteins a 1 μl of PURExpress sample was mixed with 250 nM guide G-1 (21 nt) in 10 μl of buffer containing 20 mM Bis-Tris propane, pH 8.0, 50 mM NaCl, 2 mM MgCl₂, 0.1% (v/v) Tritox X-100 and incubated for 20 minutes at 37° C. to form a pAgo/guide complex. The pAgo/G-1 complex was then combined with 50 nM 5′-FAM labeled ss DNA target T-1 in 20 μl reaction and incubated for 1 hour either at 37° C. or at 65° C. temperature. The reactions were terminated by adding an equal volume of stop buffer (95% Formamide, 0.025% Bromophenol Blue, 0.025% Xylene Cyanol, 5 mM EDTA) and heating the samples for 5 minutes at 95° C. The cleavage products were separated by gel electrophoresis on 15% denaturing polyacrylamide gel containing 7.5M urea and 24% formamide and visualized using Typhoon 9400 Scanner (GE Healthcare Chicago, Ill., USA).

Activity assays performed with purified Ago proteins were carried out with 17 nt long guides G-2 (DNA guide) and G-3 (RNA guide). Argonaute cleavage reactions were performed at 5:5:1 Ago:guide:target molar concentration ratio. For guide loading, 250 nM Ago was combined with 250 nM guide in 10 μl buffer containing 20 mM Bis-Tris propane, pH 8.0, 50 mM NaCl, 2 mM MgCl₂, 0.1% (v/v) Triton X-100 and incubated for 20 minutes at 37° C. The Ago/guide mixture was added to a 20 μl cleavage reaction containing 50 nM 5′-FAM labeled ss substrate (either T-2 or T-3), and the reaction was incubated for 1 hour at 37° C. Reactions were terminated by adding 50 mM EDTA. Cleavage products at a 4 nM final concentration were separated by Capillary Electrophoresis on an Applied Biosystems 3730xl DNA Analyzer (Applied Biosystems, Waltham, Mass., USA). The quantitative analysis of flourescent peaks was performed using PeakScanner Software v1.0 (Thermo Fisher Scientific, Inc., Waltham, Mass., USA) and fragment analysis software for in-house use at New England Biolabs as previously described (Hunt E A et al., PLoS One, 2018 Aug. 29; 13(8):e0203073; Greenough L et al., Nucl. Acids Res., 2016, 44 (2), e15).

To evaluate guide efficiency, CbAgo cleavage was examined over time by incubating a 53-nt long ss-phiX174 DNA substrate with CbAgo loaded with thirteen different 21 nt long guides for 1, 16 and 64 minutes at 37° C.

### Example 7: Argonaute Cleavage Assays on Double-Stranded DNA

A 3619-3858 nt segment of phage φX174 DNA was amplified by PCR to generate either 5′-FAM-labeled or 5′-FAM/ROX-labeled 239 bp DNA substrate. A 3393-4012 nt segment of phage φX174 DNA was amplified by PCR to generate either 5′-FAM-labeled or 5′-FAM/ROX-labeled 619 bp DNA substrate. A 3536-3858 nt segment of phage φX174 DNA was amplified by PCR with a 5′FAM- and a 5′ROX-labeled primers to generate a 5′-FAM/ROX-labeled 322 bp DNA substrate. The PCR products were purified using Monarch® PCR and DNA Cleanup kit (New England Biolabs, Inc., Ipswich, Mass.) and DNA concentration was quantified using NanoDrop spectrophotometer (ThermoFisher Scientific, Inc., Waltham, Mass., USA).

For one-strand cleavage experiments the CbAgo/guide complex was formed by incubating 0.5 μM CbAgo and IgM guide for 15 minutes at 37° C. in a 10 μl of 1× CutSmart buffer (50 mM Potassium Acetate, pH 7.9, 20 mM Tris-Acetate, 10 mM Magnesium Acetate, 100 μg/ml BSA). The CbAgo/guide complex at a 0.25/0.5 μM final concentration was combined with the 50 nM ds DNA substrate and indicated elsewhere DNA helicase in a 20 μl of 1× CutSmart buffer (New England Biolabs, Inc., Ipswich, Mass., USA) supplemented with 5 mM ATP and incubated at 37° C.

For two-strand cleavage experiments, two separate 5 μl reactions, each containing CbAgo (0.5 μM) and one guide (1 μM) were carried out to form two individual CbAgo/guide complexes that target opposite strands on ds substrate. Both CbAgo/guide complexes each at a 0.125/0.25 μM final concentration were combined with the 50 nM ds DNA substrate and indicated elsewhere DNA helicase in a 20 μl of 1× CutSmart buffer supplemented with 5 mM ATP and incubated at 37° C. The cleavage reactions were terminated by addition of 50 mM EDTA, DNA samples were diluted to 4 nM final concentration and analyzed by CE as described in EXAMPLE 6. For time course experiments the cleavage reaction volume was increased to 50 μl and 5 μl samples were withdrawn from the reaction at the indicated time points.

Cleavage reactions of φX174, pAd2_BsaI and pAd2-AvrII DNAs were carried out using 0.2 μg of DNA, which were linearized with the indicated restriction enzymes following manufacturer's recommendations (New England Biolabs, Inc., Ipswich, Mass.). The plasmids pAd2_BsaI and pAd2_AvrII were created by Dr. Richard Morgan (New England Biolabs, Inc., Ipswich, Mass.) by cloning either a 22404 bp BsaBI fragment or 19428 bp AvrII fragment of Adenovirus2 genomic DNA into pUC19 vector.

### Example 8: Helicase Assay for DNA Unwinding Activity

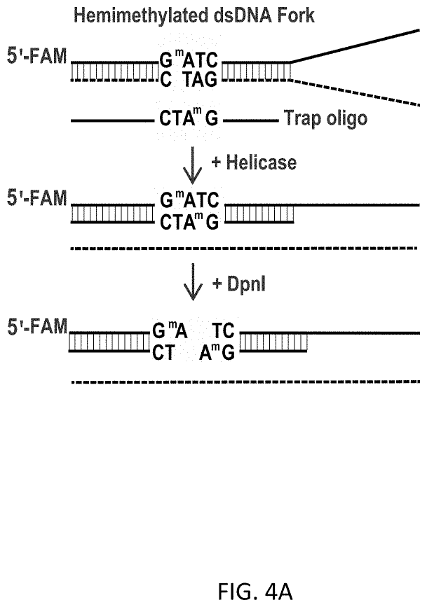

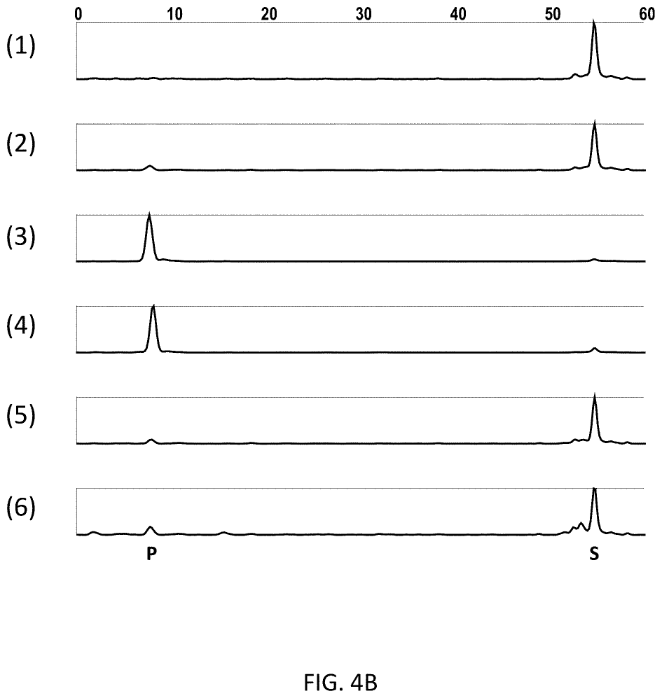

A schematic overview of an assay for monitoring helicase activity is shown in FIG. 4A. DpnI restriction endonuclease, which cleaves a fully methylated G^{m}ATC/G^{m}ATC site but does not cleave a hemimethylated site (G^{m}ATC/GATC) was used. DNA substrate comprised of 30 bp duplex portion that was extended on both strands by 30 nt-long non-complementary fork structure. A G^{m}ATC site was incorporated within the duplex portion of the 5′-FAM labeled DNA strand. The opposite strand carried an unmethylated GATC site. The fork substrate is mixed at 1:50 ratio with a 30-nt long ss trap oligo which also contains the G^{m}ATC site and is complementary to the 5′-FAM labeled strand of DNA substrate. The substrate/trap mixture is incubated with DNA helicase and DpnI. Due to the high concentration ratio of trap to substrate, the trap oligo anneals to the methylated strand as soon as helicase unwinds ds substrate, thus creating a hybrid duplex with a fully methylated site, G^{m}ATC/G^{m}ATC, which becomes a target for DpnI. The progress of DNA strand unwinding by DNA helicase can be clearly monitored by the disappearance of a 5′-FAM labeled substrate and by the accumulation of a 5′-FAM labeled DpnI cleavage product, both visualized as different size peaks obtained after resolving them by CE.

DNA oligonucleotides carrying ^{m6}A residue within a GATC site were synthesized at Organic Synthesis facility of New England Biolabs (Ipswich, Mass., USA). Double-stranded fork substrate for helicase activity assay was created by annealing two complementary ss oligonucleotides (nucleotide sequences are shown in TABLE 1). DNA unwinding reactions were carried out for 15 minutes at 37° C. in 10 μl of 1× CutSmart buffer containing 0.1 μM fork substrate, 5 μM trap oligo, 5 mM ATP, 2 units DpnI and 1 μl of serially diluted helicase. Reactions were terminated by adding 50 mM EDTA. Cleavage products were analyzed by CE as described above.

TABLE 1
Oligonucleotide Sequences

| SEQ# | Name | Sequence* | Description |
|---|---|---|---|
| | Substrates: | | |
| 8 | T-1 | 5′-FAM- | ss DNA target, |
| | | TATTTAAATAATTTAATATACTATACAACCTACTACCTCATATA | 50 nt |
| | | AATTTT | |
| 9 | T-2 | 5′-FAM- | ss DNA target, |
| | | CAGAATGGCTTGGACTCAAGTGTAAATGTACAGGGTTCAGTTT | 50 nt |
| | | TGTAATA | |
| 10 | T-3 | 5′-FAM- | ss RNA target, |
| | | CAGAAUGGCUUGGACUCAAGUGUAAAUGUACAGGGUUCA | 50 nt |
| | | GUUUUGUAAUA | |
| 11 | ss-phiX174 | 5′-FAM- | ss DNA target, |
| | | GTTTGCTGATGAACTAAGTCAACCTCAGCATCAACCTTGCGAG | 53 nt |
| | | TCATTTCTTT | |
| | Oligos for ds fork | | |
| | substrate: | | |
| 12 | Top oligo, 60 nt | 5′FAM- | Anneals to 60 |
| | | ACTCATGCAGGCATGCAGGAACGCAGTCAGATTTATGTGTCAT | nt Bottom oligo |
| | | ATAGTACGTGATTCAAG | |
| 13 | Bottom oligo, 60 nt | 5′- | Anneals to 60 |
| | | TAACGTATTCAAGATACCTCGTACTCTGTACTGACTGCGTTCCT | nt Top oligo |
| | | GCATGCCTGCATGAGT | |
| | Oligos for 50-60 bp | | |
| | substrates: | | |
| 14 | Top oligo, 60 nt | 5′-FAM- | Anneals to |
| | | ACTCATGCAGGCATGCAGGATCGCAGTCAGATTTATGTGTCAT | Bottom 1-4 |
| | | ATAGTACGTGATTCAAG | oligos |
| 15 | Bottom oligo 1, | 5′- | For Blunt |
| | 60 nt | CTTGAATCACGTACTATATGACACATAAATCTGACTGCGATCCT | substrate |
| | | GCATGCCTGCATGAGT | |
| 16 | Bottom oligo 2, | 5′- | For forked |
| | 60 nt | TAACGTATTCGTACTATATGACACATAAATCTGACTGCGATCCT | substrate |
| | | GCATGCCTGCATGAGT | |
| 17 | Bottom oligo 3, | 5′- | For substrate |
| | 50 nt | GTACTATATGACACATAAATCTGACTGCGATCCTGCATGCCTG | with 10 nt 3′-ss |
| | | CATGAGT | end |
| 18 | Bottom oligo 4, | 5′- | For substrate |
| | 70 nt | GTACTCTGTACTTGAATCACGTACTATATGACACATAAATCTGA | with 10 nt 5′-ss |
| | | CTGCGATCCTGCATGCCTGCATGAGT | end |
| | Oligos for 80 bp | | |
| | substrates: | | |
| 19 | 5′-FAM Top oligo, | 5′FAM- | Anneals to 80 nt |
| | 80 nt | GCTAGATAGATTGACAGATTACTCAAGCAGGCATGCAGGATC | Bottom to |
| | | GCAGTCAGATTTATGTGTCATATAGTACGTGATTCAAG | make blunt end |
| 20 | Bottom oligo, | 5′- | Anneals to 80 nt- |
| | 80 nt | CTTGAATCACGTACTATATGACACATAAATCTGACTGCGATCCT | FAM to make |
| | | GCATGCCTGCTTGAGTAATCTGTCAATCTATCTAGC | blunt substrate |
| 21 | 5′-FAM Top oligo 1, | 5′FAM- | Anneals to 80 nt |
| | 90 nt | GCTAGATAGATTGACAGATTACTCAAGCAGGCATGCAGGATC | Bottom to |
| | | GCAGTCAGATTTATGTGTCATATAGTACGTGATTCAAGTACAG | make lOnt 3′-ss |
| | | AGAAC | end |
| 22 | Bottom oligo 1, | 5′- | Anneals to 80 nt- |
| | 90 nt | GTTCTCTGTACTTGAATCACGTACTATATGACACATAAATCTGA | FAM Top to |
| | | CTGCGATCCTGCATGCCTGCTTGAGTAATCTGTCAATCTATCTA | make lOnt 5′-ss |
| | | GC | end |
| 23 | 5′-FAM Top oligo 2, | 5′FAM- | Anneals to 90 nt |
| | 90 nt | GCTAGATAGATTGACAGATTACTCAAGCAGGCATGCAGGATC | Bottom2 to |
| | | GCAGTCAGATTTATGTGTCATATAGTACGTGATTCAAGTACAG | make forked |
| | | AGAAC | end |
| 24 | Bottom oligo 2, | 5′- | Anneals to |
| | 90 nt | TAAGGTATTCCTTGAATCACGTACTATATGACACATAAATCTG | 90 nt-FAM Top2 |
| | | ACTGCGATCCTGCATGCCTGCTTGAGTAATCTGTCAATCTATCT | to make forked |
| | | AGC | end |
| | Guides: | | |
| 25 | B12 | 5′/phos/TGACTGCGATCCTGCAT | DNA guide, 17 |
| | | | nt |
| 26 | 17 nt guide | 5′/phos/TGACTGCGTTCCTGCAT | DNA guide, 17 |
| | | | nt |
| 27 | G-1 | 5′/phos/TGAGGTAGTAGGTTGTATAGT | DNA guide, 21 |
| | | | nt |
| 28 | G-2 | 5′/phos/TGAACCCTGTACATTTA | DNA guide, 17 |
| | | | nt |
| 29 | G-3 | 5′/phos/UGAACCCUGUACAUUUA | RNA guide, 17 |
| | | | nt |
| 30 | T1 | 5′/phos/TCGCAAGGTTAGTGCTGAGGT | DNA guide, 21 |
| | | | nt |
| 31 | T1-C1 | 57phos/CGCAAGGTTAGTGCTGAGGTT | DNA guide, 21 |
| | | | nt |
| 32 | T1-G1 | 5′/phos/GCAAGGTTAGTGCTGAGGTTG | DNA guide, 21 |
| | | | nt |
| 33 | T1-C2 | 5′/phos/CAAGGTTAGTGCTGAGGTTGA | DNA guide, 21 |
| | | | nt |
| 34 | T1-A1 | 5′/phos/AAGGTTAGTGCTGAGGTTGAC | DNA guide, 21 |
| | | | nt |
| 35 | T1-A2 | 5′/phos/AGGTTAGTGCTGAGGTTGACT | DNA guide, 21 |
| | | | nt |
| 36 | T1-G2 | 5′/phos/GGTTAGTGCTGAGGTTGACTT | DNA guide, 21 |
| | | | nt |
| 37 | T1-G3 | 5′/phos/GTTAGTGCTGAGGTTGACTTA | DNA guide, 21 |
| | | | nt |
| 38 | T2 | 5′/phos/TTAGTGCTGAGGTTGACTTAG | DNA guide, 21 |
| | | | nt |
| 39 | T2-T2 | 5′/phos/TAGTGCTGAGGTTGACTTAGT | DNA guide, 21 |
| | | | nt |
| 40 | T2-A1 | 5′/phos/AGTGCTGAGGTTGACTTAGTT | DNA guide, 21 |
| | | | nt |
| 41 | T2-G1 | 5′/phos/GTGCTGAGGTTGACTTAGTTC | DNA guide, 21 |
| | | | nt |
| 42 | T3 | 5′/phos/TGCTGAGGTTGACTTAGTTCA | DNA guide, 21 |
| | | | nt |
| 43 | B1 | 5′/phos/TGAACTAAGTCAACCTCAGCA | DNA guide, 21 |
| | | | nt |
| 44 | B2 | 5′/phos/TAAGTCAACCTCAGCACTAAC | DNA guide, 21 |
| | | | nt |
| 45 | B3 | 5′/phos/TCAACCTCAGCACTAACCTTG | DNA guide, 21 |
| | | | nt |
| 46 | B4 | 5′/phos/TCAGCACTAACCTTGCGAGTC | DNA guide, 21 |
| | | | nt |
| 47 | XhoT | 5′/phos/TTTGGTCTAACTTTACCGCTA | DNA guide, 21 |
| | | | nt |
| 48 | XhoB | 5′/phos/TTAGTAGCGGTAAAGTTAGAC | DNA guide, 21 |
| | | | nt |
| 49 | Sap21F | 5′/phos/TGGTCAGATTGGTCGTCTTAT | DNA guide, 21 |
| | | | nt |
| 50 | SapB | 5′/phos/TAAGACGACCAATCTGACCAG | DNA guide, 21 |
| | | | nt |
| 51 | StuT | 5′/phos/TGATGGAACTGACCAAACGTC | DNA guide, 21 |
| | | | nt |
| 52 | StuB | 5′/phos/TAACGACGTTTGGTCAGTTCC | DNA guide, 21 |
| | | | nt |
| 53 | AdB-450T | 5′/phos/TCAAGCAAGTGTCTTGCTGTC | DNA guide, 21 |
| | | | nt |
| 54 | AdB450B | 5′/phos/TAAAGACAGCAAGACACTTGC | DNA guide, 21 |
| | | | nt |
| 55 | AdA-20860T | 5′/phos/TGTCATCTTTTAAGCTTGCCT | DNA guide, 21 |
| | | | nt |
| 56 | AdA-20860B | 5′/phos/TACCAGGCAAGCTTAAAAGAT | DNA guide, 21 |
| | | | nt |
| 57 | T5 | 5′/phos/TAGGTATCTCAGTTCGGTGTA | DNA guide, 21 |
| | | | nt |
| 58 | B5 | 5′/phos/AACGACCTACACCGAACTGAG | DNA guide, 21 |
| | | | nt |
| | PCR Primers: | | |
| 59 | 239-Fw | 5′-GCCAAATGCTTACTCAAGCTCAAAC | PCR primer for |
| | | | 239 bp |
| | | | substrate, 25 nt |
| 60 | 239-ROX-Fw | 5′/ROX/GCCAAATGCTTACTCAAGCTCAAAC | PCR primer for |
| | | | 239 bp |
| | | | substrate, 25 nt |
| 61 | 239-FAM-Rv | 5′/FAM/TCCAAGTATCGGCAACAGCTTTAT | PCR primer for |
| | | | 239 bp |
| | | | substrate, 24 nt |
| 62 | 619-Fw | 5′-CTAAAATGCAACTGGACAATCAGAAAGAGA | PCR primer for |
| | | | 619 bp |
| | | | substrate, 30 nt |
| 63 | 619-ROX-Fw | 5′/ROX/CTAAAATGCAACTGGACAATCAGAAAGAGA | PCR primer for |
| | | | 619 bp |
| | | | substrate, 30 nt |
| 64 | 619-FAM-Rv | 5′/FAM/TGACATTCAGAAGGGTAATAAGAACGAAC | PCR primer for |
| | | | 619 bp |
| | | | substrate, 29 nt |
| 65 | 322-Fw | 5′/ROX/CAGAAGGAGTCTACTGCTCGCGT | PCR primer for |
| | | | 322 bp |
| | | | substrate, 23 nt |
| 66 | 322-Rv | 5′/FAM/TCCAAGTATCGGCAACAGCTTTAT | PCR primer for |
| | | | 322 bp |
| | | | substrate, 24 nt |
| 67 | phiX174-ROX-T1-5 | 5′/ROX/ATGACTCGCAAGGTTAGTGCTGA | PCR primer for |
| | | | 332 bp DNA1, |
| | | | 23 nt |
| 68 | phiX174-FAM-Rv | 5′/FAM/TGACATTCAGAAGGGTAATAAGAACGAAC | PCR primer for |
| | | | 332 bp DNA1, |
| | | | 29 nt |
| 69 | pUC19-300-Fw | 5′/ROX/ACATGTGAGCAAAAGGCCAGCAAAA | PCR primer for |
| | | | 300 bp DNA2, |
| | | | 25 nt |
| 70 | pUC19-300-Rv | 5′/FAM/AGCGAACGACCTACACCGAACTG | PCR primer for |
| | | | 300 bp DNA2, |
| | | | 23 nt |
| 71 | pET28c-Fw4 | 5′TGAGATCCGGCTGCTAACAAAG | PCR primer for |
| | | | pET28c |
| | | | fragment, 22 nt |
| 72 | pET28c-Rv | 5′CATATGGCTGCCGCGCGGCAC | PCR primer for |
| | | | pET28c |
| | | | fragment, 21 nt |
| 73 | B1-nhis-Fw | 5′GCGCGGCAGCCATATGAGTGATGTCGCCGAGACA | PCR primer for |
| | | | recB B1 |
| | | | fragment, 34 nt |
| 74 | B1-A1020-RV | 5′AACTCCATCGCCACCTGTTTATTGCGG | PCR primer for |
| | | | recB B1 |
| | | | fragment, 27 nt |
| 75 | B2-A1020-FW | 5′ACAGGTGGCGATGGAGTTTTATCTGCCGA | PCR primer for |
| | | | recB B2 |
| | | | fragment, 29 nt |
| 76 | B2-A1080-FW | 5′TGCTCGCCTATGCATCCAACTGGTTGGGTGAA | PCR primer for |
| | | | recB B2 |
| | | | fragment, 32 nt |
| 77 | B3-A1080-RV | 5′TTGGATGCATAGGCGAGCAGGTAATAACGCCCT | PCR primer for |
| | | | recB B3 |
| | | | fragment, 33 nt |
| 78 | B3-end-Rv | 5′GTTAGCAGCCGGATCTCATTACGCCTCCTCCAGGGTCAT | PCR primer for |
| | | | recB B3 |
| | | | fragment, 39 nt |
| | Helicase assay: | | |
| 79 | Top strand, | 5′/FAM/ACTCATGCAGGCATGCAGG/m6A/TCGCAGTCAGATT | Anneals to |
| | 60 nt | TATGTGTCATATAGTACGTGATTCAAG | bottom strand |
| 80 | Bottom strand, | 5′TAACGTATTCAAGATACCTCGTACTCTGTACTGACTGCGATC | Anneals to top |
| | 60 nt | CTGCATGCCTGCATGAGT | strand |
| 81 | Trap oligo, | 5′CTGACTGCG/m6A/TCCTGCATGCCTGCATGAGT | Anneals to top |
| | 30 nt | | strand |
| | Oligos for 29 nt ds | | |
| | bridge oligo: | | |
| 82 | Top linker oligo | 5′/phos/GTAGGTCGTTCGCTATGACTCGCAAGGTTAGTGCTG | Anneals to |
| | 37 nt | A | bottom linker |
| | | | oligo |
| 83 | Bottom linker oligo, | 5′/phos/AACCTTGCGAGTCATAGCGAACGACCTACACCGAAC | Anneals to top |
| | 37 nt | T | linker oligo |

*Nucleotides in PCR primers that were changed to introduce mutations in the recB gene are underlined.

### Example 9: Ligation of CbAgo Cleaved DNA Fragments

A 332 bp 5′FAM/ROX-labeled DNA1 was generated by PCR using a3681-4012 nt segment of φX174 DNA as a template. DNA 1 was cleaved with CbAgo loaded with guides T2+B1 to generate a 5′-ROX labeled 15 bp, and a 5′-FAM labeled 309 bp cleavage products. A 300 bp 5′FAM/ROX-labeled DNA2 was generated by PCR using an 806-1105 nt segment of pUC19 DNA as a template. DNA2 was cleaved with CbAgo loaded with guides T5+B5 to generate a 5′-ROX labeled 14 bp, and a 5′-FAM labeled 278 bp cleavage products. All CbAgo cleavage products were flanked with 8 nt long 3′-ss extensions. The 15 bp and 14 bp fragments were discarded by column purification with Monarch® PCR and DNA Cleanup Kit (New England Biolabs, Inc., Ipswich, Mass.).

A 29 bp ds “Bridge” oligonucleotide flanked with an 8-nt long 3′-ss extensions on both ends was created by combining two complementary 5′-phophorylated ss oligonucleotides (1 nmol each) in 100 μl of 10 mM Tris-HCl buffer, pH 7.5 and heating for 5 min at 95° C. followed by slow cooling down to room temperature. DNA fragment ligation was carried out with 400 units of T4 DNA ligase (New England Biolabs, Inc., Ipswich, Mass., USA) in 10 μl of T4 DNA ligase buffer. First, 0.3 pmols of 309 bp DNA1 fragment and 0.3 pmols of 29 bp synthetic Bridge oligonucleotide were ligated for 15 minutes at 25° C. The reaction was then supplemented with 0.3 pmols of 278 bp DNA2 fragment and ligation continued for another 15 minutes at 25° C. Ligation products were analyzed by CE.

### Example 10: Bioinformatics Analysis for Identifying Mesophilic Agos

Candidate Argonaute proteins were identified in a series of steps. First off, three known Argonaute proteins TtAgo (UniProt ID Q746M7), NgAgo (UniProt ID LOAJX6), and PfAgo (UniProt ID Q8U3D2) were aligned using MUSCLE multiple sequence alignment software (Edgar R C, 2004, Nucleic Acids Res. 32(5):1792-1797). The resulting multiple sequence alignment was used as an input to PSI-BLAST to search against UniProt database. The expected threshold was set at 1×10⁻⁴, and PSI-BLAST was running multiple iterations until convergence. All bacterial homologs found were extracted based on taxonomic classification. Only proteins containing PAZ and PIWI domains were considered for further analysis. The presence of catalytic PIWI and PAZ domains in found homologs was checked by running HMM search using domain profiles available in PFAM database (PFAM PF02171 and PF02170 for PIWI and PAZ domain, respectively). PAZ domain profile in PFAM is built mainly on sequences of eukaryotic proteins and resulted in a very few hits when run against bacterial proteins. Therefore, the HMM profile for bacterial PAZ domain was generated from scratch using HMMER software (Eddy S R, 2011, PLOS Comp. Biol., 7:e1002195) based on sequences of known Argonaute proteins. Proteins originating from known thermophilic organisms were discarded. Additionally, only proteins that share less than 90% sequence identity to each other were selected for further analysis. Finally, proteins that do not contain aspartates at conserved PIWI catalytic sites were excluded. The remaining list of forty-five bacterial Argonautes is shown in TABLE 2.

TABLE 2
Bacterial Argonautes

| GenBank | Organism | Argonaute |
|---|---|---|
| CAI09102.1 | Aromatoleum aromaticum EbN1 | ArAgo* |
| WP_091870084.1 | Butyrivibrio sp. INlla16 | BsAgo |
| WP_058142162.1 | Clostridium butyricum | CbAgo |
| WP_055276084.1 | Clostridium disporicum | CdAgo |
| EHP50500.1 | Clostridium perfringens WAL-14572 | CpAgo |
| WP_016205751.1 | Clostridium sartagoforme | CsAgo |
| WP_042399050.1 | Clostridium saudiense | CaAgo* |
| WP_055195547.1 | Dorea longicatena | DlAgo |
| WP_021982449.1 | Eubacteriaceae bacterium | EbAgo |
| WP_009456812.1 | Fischerella thermalis | FtAgo |
| WP_055268308.1 | Fusicatenibacter saccharivorans | FsAgo |
| WP_055087491.1 | Intestinibacter bartlettii | IbAgo |
| WP_010289662.1 | Kurthia massiliensis | KmAgo |
| WP_009787621.1 | Lyngbya sp. PCC 8106 | LsAgo |
| WP_017841076.1 | Methylomicrobium buryatense | MbAgo |
| WP_061432364.1 | Microcystis aeruginosa | MaAgo |
| KKI99518.1 | Prochlorothrix hollandica | PhAgo |
| | PCC 9006 = CALU 1027 | |
| CCQ09996.1 | Pseudoalteromonas luteoviolacea | PuAgo* |
| | B = ATCC 29581 | |
| WP_090164194.1 | Pseudobutyrivibrio xylanivorans | PxAgo |
| WP_100217698.1 | Pseudomonas luteola | PlAgo |
| WP_045054463.1 | Aliterella atlantica | |
| WP_015213401.1 | Anabaena cylindrica | |
| WP_039723517.1 | Aphanocapsa montana | |
| AFY32165.1 | Calothrix sp. PCC 7507 | |
| WP_015159126.1 | Chamaesiphon minutus | |
| WP_015329024.1 | Chamaesiphon minutus | |
| WP_015201773.1 | Crinalium epipsammum | |
| WP_034404411.1 | Deinococcus sp. RL | |
| WP_034806158.1 | Exiguobacterium sp. AB2 | |
| WP_013324485.1 | Gloeothece verrucosa | |
| WP_058998822.1 | Leptolyngbya sp. NIES-2104 | |
| WP_027844945.1 | Mastigocoleus testarum | |
| WP_006630972.1 | Microcoleus vaginatus | |
| WP_015150617.1 | Oscillatoria acuminata | |
| WP_042211195.1 | Paenibacillus borealis | |
| WP_015118671.1 | Rivularia sp. PCC 7116 | |
| WP_039715179.1 | Scytonema millei | |
| WP_015195532.1 | Stanieria cyanosphaera | |
| WP_011244830.1 | Synechococcus elongatus | |
| WP_011434286.1 | Synechococcus sp. JA-2-3B′a(2-13) | |
| ABD00306.1 | Synechococcus sp. JA-3-3Ab | |
| WP_062436241.1 | Synechococcus sp. PCC 73109 | |
| WP_028947682.1 | Synechocystis sp. PCC 6714 | |
| WP_015880612.1 | unclassified Exiguobacterium | |
| WP_012306644.1 | unclassified Synechococcus | |

*Argonaute is named after the bacterium from which it was isolated, using a naming system based on the first letters of bacterial genus and bacterial species. The second letter of the species is used in cases when Argonaute with the identical name already used. For example, previously discovered Argonaute from Aquifex aeolicus was named AaAgo (Yuan YR et al., Mol.Cell. 2005 Aug. 5;19(3):405-19), therefore the name ArAgo is used to name the Argonaute from Aromatoleum aromaticum EbN1.

### Example 11: Identifying Mesophilic DNA-Guided DNA-Cleaving Argonautes

From the compiled list of 45 bacterial Agos, 20 candidates residing in mesophilic bacterial hosts were selected for screening of catalytically active proteins. The names of selected pAgos, bacterial hosts and NCBI Gene bank Accession numbers are listed in TABLE 3. The quick examination of pAgo activity was performed employing proteins expressed with PURExpress® In Vitro Protein Synthesis kit (New England Biolabs, Inc., Ipswich, Mass.). SDS-PAGE analysis confirmed detectable levels of soluble proteins in case of 19 pAgo candidates, except for CbAgo protein from C. butyricum which was found in an insoluble fraction (data not shown). Ten out of twenty tested pAgo candidates revealed DNA-guided DNA cleavage activity at 37° C. and total 13 candidates were active at 65° C. (FIG. 1). Four pAgo candidates displaying highest cleavage activity at 37° C. and 65° C. were found in the hosts that belong to the Clostridium genus. CbAgo showed weak activity at 37° C., but it was attributed to the low levels of soluble protein in PURExpress reaction.

TABLE 3
Bacterial hosts and NCBI Gene bank
Accession numbers of pAgo proteins

| Argonaute | Bacteria | Gene Bank ID# |
|---|---|---|
| ArAgo* | Aromatoleum aromaticum EbN1 | CAI09102.1 |
| BsAgo | Butyrivibrio species INlla16 | WP_091870084.1 |
| CbAgo | Clostridium butyricum | WP_058142162.1 |
| CdAgo | Clostridium disporicum | WP_055276084.1 |
| CpAgo | Clostridium perfringens | EHP50500.1 |
| | WAL-14572 | |
| CsAgo | Clostridium sartagoforme | WP_016205751.1 |
| CaAgo* | Clostridium saudiense | WP_042399050.1 |
| DlAgo | Dorea longicatena | WP_055195547.1 |
| EbAgo | Eubacteriaceae bacterium | WP_021982449.1 |
| FtAgo | Fischerella thermalis | WP_009456812.1 |
| FsAgo | Fusicatenibacter saccharivorans | WP_055268308.1 |
| IbAgo | Intestinibacter bartlettii | WP_055087491.1 |
| KmAgo | Kurthia massiliensis | WP_010289662.1 |
| LsAgo | Lyngbya species PCC 8106 | WP_009787621.1 |
| MbAgo | Methylomicrobium buryatense | WP_017841076.1 |
| MaAgo | Microcystis aeruginosa | WP_061432364.1 |
| PhAgo | Prochlorothrix hollandica | KKI99518.1 |
| | PCC 9006 = CALU 1027 | |
| PlAgo | Pseudomonas luteola | WP_100217698.1 |
| PuAgo* | Pseudoalteromonas luteoviolacea | CCQ09996.1 |
| | B = ATCC 29581 | |
| PxAgo | Pseudobutyrivibrio xylanivorans | WP_090164194.1 |

*Argonaute is named after the bacterium from which it was isolated, using a naming system based on the first letters of bacterial genus and bacterial species. The second letter of the species is used in cases when Argonaute with the identical name already used.

Six pAgos were purified and characterized with a goal to find an Argonaute exhibiting the highest cleavage activity at 37° C. Five selected pAgos originated from hosts assigned to the Clostridium genus. The host for IbAgo also originally was assigned as Clostridium bartlettii, but later the species was re-assigned as Intestinibacter bartlettii (Song Y. L. et al. 2004, Anaerobe, 10(3), 179-184; Gerritsen, J. et al., 2014, J. Syst. Evol. Microbiol, 64(Pt5), 1600-1616). A previously described high-throughput capillary gel electrophoresis-based activity assay was used (Hunt E A et al., PLoS One, 2018 Aug. 29; 13(8):e0203073; 31, Greenough L et al., Nucl. Acids Res., 2016, 44 (2), e15) to rapidly characterize the purified pAgos for guide preference (DNA vs RNA guide) and target preference (DNA vs RNA target). The obtained results are summarized in TABLE 4. All tested pAgos preferred DNA guides and DNA targets over RNA guides and RNA targets. CpAgo was the only Argonaute capable of DNA-guided cleavage of RNA and RNA-guided cleavage of DNA, albeit at a significantly reduced rates when compared to the DNA-guided DNA-target cleavage. The majority of investigated pAgos were capable of cleaving DNA at temperatures spanning from 30° C. up to 75° C. The highest activity was observed at 55-65° C., except for IbAgo which was the most active at 45° C. (data not shown). This suggests that the investigated pAgos still prefer higher temperatures for the optimal catalysis even if they adapted to function in mesophilic hosts. The efficiency of DNA target cleavage at 37° C. was evaluated at different time points ranging from 5 to 120 minutes. The results presented in FIG. 2 demonstrate that CbAgo cleaves DNA targets much faster that other tested pAgos and yields close to complete target cleavage levels in 20-40 minutes at 37° C. Based on target cleavage efficiency at 37° C. the investigated pAgos were ranked in the following order: CbAgo>CpAgo>CsAgo>IbAgo>CaAgo>CdAgo. Because of the robust activity the CbAgo was selected for forward studies aimed at finding reaction settings that support double-stranded DNA cleavage at 37° C. temperature.

TABLE 4
Enzymatic properties of Ago Proteins

| | | DNA-Guided Cleavage of | | RNA-Guided Cleavage of | |
|---|---|---|---|---|---|
| Species | Argonaute | DNA | RNA | DNA | RNA |
| Clostridium perfringens | CpAgo | +++ | ++ | + | − |
| Clostridium disporicum | CdAgo | +++ | − | − | − |
| Clostridium butyricum | CbAgo | +++ | − | − | − |
| Clostridium sartagoforme | CsAgo | +++ | − | − | − |
| Clostridium saudiense | CaAgo | +++ | − | − | − |
| Clostridium bartlettii | IbAgo | +++ | − | − | − |

### Example 12: The Effect of Guide Nucleotide Sequence on Cleavage Efficiency of CbAgo

An array of 13 different guides of 21 nt in length were designed to hybridize with a 53-nt long ss DNA substrate at positions that were shifted by one nucleotide to the right with respect to each other (FIG. 3A). Such guide arrangement allowed to shift the cleavage position of CbAgo by one nucleotide along the substrate DNA. The efficiency of target cleavage by each CbAgo/guide complex was evaluated after 1-, 4- and 16-minute incubation at 37° C. Results presented in FIG. 3B show that efficacy of the guides may change substantially when they are moved by one nucleotide along the target sequence. For example, CbAgo loaded with guide T1-C1 or guide T2-A1 could cleave the target only to 50% or 35% completion in 16 minutes, respectively (FIG. 3B). The target cleavage was not completed even after 64 minutes (data not shown). However, by shifting T1-C1 guide sequence by one nucleotide in the 3′ direction along the target sequence produced a very efficient guide T1 which in 4 minutes increased CbAgo cleavage up to 74%. Similarly, by shifting the T2-A1 guide sequence by one nucleotide in the 5′ direction along DNA substrate generated a very efficient guide T2-GI which in 4 minutes increased CbAgo cleavage up to 80%. But after 64-minute incubation at 37°, the majority of guides were capable to direct CbAgo for a specific ss target cleavage despite the observed clear differences in guide efficiencies at earlier time points.

### Example 13: Search for Accessory Proteins that can Help Mesophilic Argonautes to Separate Ds DNA Strands at 37° C.

A variety of proteins which have an ability either to intercalate into ds DNA or to stabilize ss DNA regions during various cell processes, such as DNA replication, DNA recombination and DNA repair, were tested in combination with CbAgo for ds DNA cleavage at 37° C. The tested proteins include E. coli RecA protein (New England Biolabs, Inc., Ipswich, Mass.) which catalyzes the pairing of ssDNA with complementary regions of dsDNA; Spy dCas9 protein (New England Biolabs, Inc., Ipswich, Mass.), an inactive mutant of Cas9 nuclease, which retains ability to intercalate duplex DNA for programmable DNA binding activity; and three single-stranded DNA binding proteins, T4 gp32, extreme thermostable single-stranded DNA binding protein ET SSB (T4 gp32 and ET SSB, New England Biolabs, Inc., Ipswich, Mass., USA), and E. coli SSB (ThermoFisher Scientific, Inc. Waltham, Mass., USA), which can bind to ss DNA regions thus preventing hybridization of complementary DNA strands. However, neither of the above listed accessory proteins were capable of increasing CbAgo cleavage activity on ds DNA targets at 37° C.

The possibility was explored that unwinding of ds DNA substrates by mesophilic helicases might help CbAgo to access single-stranded targets at 37° C. The commercially available mesophilic DNA helicases, EcoRecQ (Abcam, Inc., Cambridge, Mass., USA), EcoUvrD (MyBioSource, Inc. San Diego, Calif., USA), T4 gp41 (GoldBio Inc., St. Louis Mo., USA) and T4 DNA Helicase (McLab, Inc., South San Francisco, Calif., USA) first were screened for their ability to unwind forked ds DNA. A schematic overview of assay which was used to test these helicases for DNA unwinding activity is shown in FIG. 4A and a detail protocol is outlined in EXAMPLE 8. Among four commercial helicases only EcoRecQ and T4 DNA Helicase displayed a robust DNA unwinding activity (FIG. 4B). The low DNA unwinding activity of Eco UvrD or T4 gp41 helicases is consistent with observations that these helicases require self-assembly and interaction with accessory proteins to activate their unwinding activity in vitro. Close inspection of T4 DNA Helicase obtained from McLab, Inc. and T4 gp41 helicase obtained from GoldBio, Inc. revealed that they display different protein sizes on SDS-PAGE. A proteomic analysis showed that T4 DNA Helicase amino acid sequence is identical to Dda helicase from T4-like phage Slur07, therefore this helicase is further referred to as Slur07 Dda.

A capillary electrophoreses-based assay was employed to test if EcoRecQ and Slur07Dda DNA helicases can help CbAgo in cleaving ds DNA targets at 37° C. A schematic overview of the assay is shown in FIG. 5A and is described in detail in EXAMPLE 7. The assay is based on the principle that as soon as helicase unwinds DNA strands CbuAgo/guide complex can cleave ss target prior to strand re-annealing may occur. Initially, a DNA substrate comprised of 30 bp long duplex region flanked with 30 nt-long ss extensions on both strands (fork structure) was used in this assay as it was shown to be the best substrate for various DNA helicases. The 21 nt guide was complement to the sequence within a 30 bp duplex portion of the 5′-FAM labeled strand and DNA-guided cleavage by CbAgo was expected to generate a 16-nt long 5′-FAM labeled ss product. CbAgo at first is pre-incubated with a guide at 1:2 molar concentration ratio to form a CbAgo/guide complex. The complex is then combined with ds DNA substrate and DNA helicase and the reaction is incubated at 37° C. No ds substrate cleavage takes place when only CbAgo/guide complex is added to the reaction indicating that CbAgo is unable to initiate DNA-guided cleavage within a 30 bp portion of ds DNA (FIG. 5, LANE 2). However, in the presence of either EcoRecQ or Slur07Dda helicases CbAgo/guide was capable of cleaving DNA target in a guide-specific mode as indicated by disappearance of the uncut substrate strand and by the appearance of short cleavage product (FIG. 5, LANES 3 and 4).

In summary the obtained results confirmed that DNA helicases have a potential to act as accessory proteins in helping mesophilic Argonautes to access the single-stranded targets within a duplex DNA at 37° C.

### Example 14: Characterization of CpeRecQ and CbuRecQ DNA Helicases

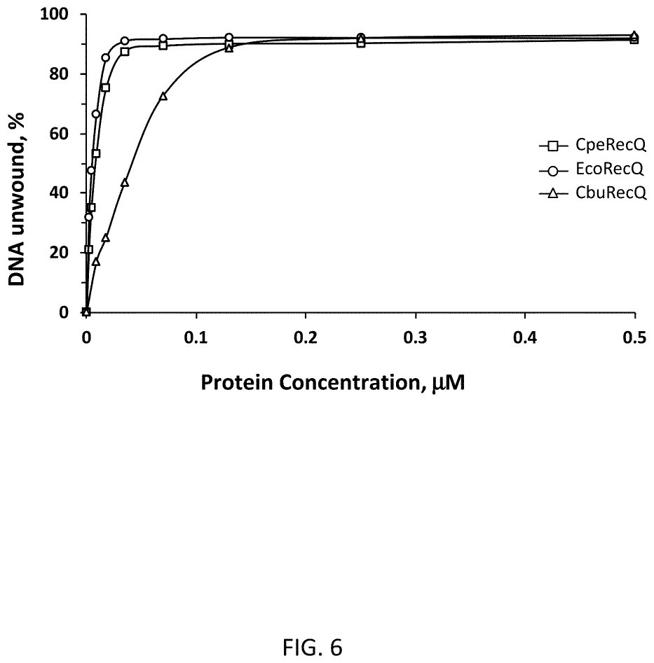

RecQ helicases are highly conserved among bacteria throughout evolution as they are important for the maintenance of genome stability. Using the known protein sequence of EcoRecQ as a reference, we have identified putative RecQ-like DNA helicases in Clostridium butyricum (WP_003411240.1) and Clostridium perfringens (WP_011590145.1) which also are the hosts for CbAgo and CpAgo, respectively. Detailed protein sequence comparison showed that CbuRecQ and CpeRecQ share strong similarity with EcoRecQ at the N-terminal protein region where reside the conserved helicase, RecQ-Ct and HRDC domains (Morozov V. et al., Trends Biochem. Sci., 1997, 22(11), 417-418). However, CbuRecQ and CpeRecQ proteins have an additional C-terminal domain of unknown function that is missing in EcoRecQ. The genes encoding EcoRecQ, CpeRecQ and CbuRecQ helicases were ordered in pET28c vector from GenScript (Piscataway, N.J., USA). The purified RecQ proteins were compared for DNA unwinding activity. The activity assay permitted to confirm that both putative proteins, CpeRecQ and CbuRecQ were functional DNA helicases. The comparison of three RecQ proteins indicated that EcoRecQ and CpeRecQ possess similar specific activity (FIG. 6). In contrast, CbuRecQ was less efficient as approximately 8-fold higher protein concentration was necessary to reach a maximum level of DNA substrate unwinding (FIG. 6).

As seen from the results presented in FIG. 5, the ability of CbAgo to cleave ds DNA relies on efficient DNA strand unwinding by DNA helicase. Most DNA helicases require single-stranded tails as an initiation point to start unwinding DNA duplex, and exhibit either 3′→5′ or 5′→3′ unwinding polarity. For example, RecQ is a 3′→5′ DNA helicase which prefers 3′-ss tails, whereas Dda is a 5′→3′ DNA helicase and prefers 5′-ss tails. RecQ family helicases have been shown to be capable of unwinding a wide array of DNA duplexes, including a fully paired ds DNA. For this reason, it must be considered that CbAgo cleavage might rely on a different helicase in each case when ds DNA substrate is flanked by a different end structure.

A series of experiments were carried out to test double-stranded cleavage activity of CbAgo in the presence of Slur07 Dda, EcoRecQ and CpeRecQ DNA helicases. In addition, the CbAgo cleavage was tested employing DNA helicase mixtures made up by combining either EcoRecQ or CpeRecQ with Slur07 Dda helicase. Cleavage efficiency was compared using two sets of four double-stranded substrates. In one set, the substrates carried a common 50-60 bp duplex region (FIG. 7A). The substrates in the second set carried a common 80 bp duplex region (FIG. 8A). Three substrates in each set were flanked with 10-nt long single-stranded regions on one or both strands, whereas the fourth substrate was fully double-stranded. DNA substrates were created by combining two complementary oligonucleotides (1 nmol each) in 100 μl of 10 mM Tris-HCl buffer, pH 7.5 and heating for 5 min at 95° C. followed by slow cooling down to room temperature. CbAgo/guide complex was formed by incubating 0.5 μM CbAgo and 0.5 μM guide B12 for 15 minutes at 37° C. in a 10 μl of 1× CutSmart buffer (50 mM Potassium Acetate, pH 7.9, 20 mM Tris-Acetate, 10 mM Magnesium Acetate, 100 μg/ml BSA). The CbAgo/B12 complex at a 0.25/0.25 μM final concentration was combined with either 5 μM RecQ or with 0.6 μM Slur07 Dda helicase and 50 nM of 5′-FAM labeled DNA substrate in a 20 μl of 1× CutSmart buffer supplemented with 5 mM ATP and incubated for 1 hour at 37° C. The cleavage reactions were terminated by addition of 50 mM EDTA, DNA samples were diluted to 4 nM final concentration and analyzed by CE as described in EXAMPLE 6.

Among the set of substrates with 50-60 bp duplex portion (FIG. 7A), the CbAgo cleavage was most efficient on Substrate 1 implying that employed DNA helicases were able to efficiently unwind duplex DNA tailed with a fork structure. As projected, on a 3′-tailed ds DNA (Substrate 2) CbAgo activity was higher in the presence of RecQ helicases, whereas on a 5′-tailed ds DNA (Substrate 3) CbAgo revealed higher activity in the presence of Slur07 Dda helicase (FIG. 7B). In the presence of any of the three DNA helicases CbAgo displayed a substantial cleavage activity on blunt-ended ds DNA (Substrate 4) indicating that both RecQ and Slur07 Dda helicases were capable of unwinding blunt-ended DNA duplex. CbAgo cleavage of blunt-ended substrate was even more efficient when a mixture of two DNA helicases (either EcoRecQ+Slur07Dda or CpeRecQ+Slur07Dda) was used for strand unwinding (FIG. 7B). These results suggest that CbAgo activity on ds DNA may be significantly enhanced if DNA duplex unwinding is carried out simultaneously by several helicases.

CbAgo activity declined when duplex DNA portion was increased from 50-60 bp to 80 bp suggesting that RecQ and Dda helicases were not able to efficiently unwind 80 bp DNA duplex (FIG. 8B). These results indicate that unwinding processivity of RecQ and Dda helicases may not support CbAdo cleavage of longer DNA targets under the conditions tested. Yet, the highest level of CbAgo activity on any 80-bp long DNA substrate was observed in the presence of CpeRecQ+Slur07 Dda helicase mixture (FIG. 8B), thus, implying that CbAgo has a potential to cleave longer ds DNA substrates, but a highly processive and a highly efficient DNA helicase compatible with CbAgo activity must be identified.

### Example 15: Characterization of RecB^{exo-}C DNA Helicase

Crystal structure of RecBCD enzymes revels four conserved residues (Glu1020, Asp1067, Asp1080 and Lys 1082) in the active site of the RecB nuclease domain (Singleton M R et al., Nature, 2004, 432, 187-193. doi: 10.1038/nature02988). Nuclease deficient variant, referred to as RecB^{exo-}, was constructed by replacing three catalytic residues of the nuclease domain, E1020, D1080 and K1082 with an alanine residue. The purified RecB^{exo-} and RecC subunits were mixed at 1:1 stoichiometry to the final 10 μM concentration to reconstitute RecB^{exo-}C helicase.

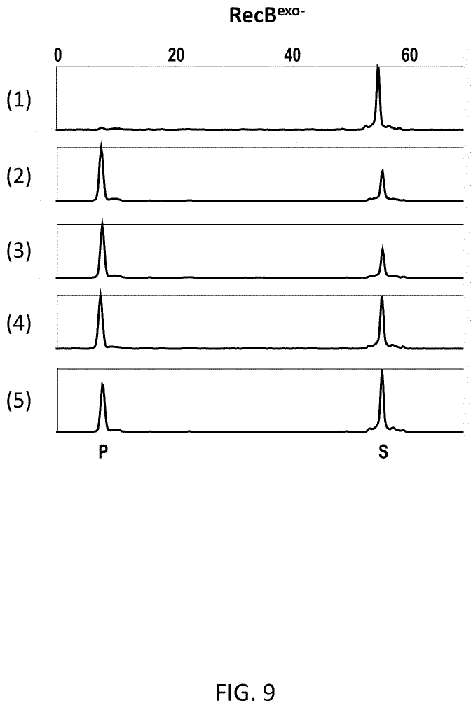

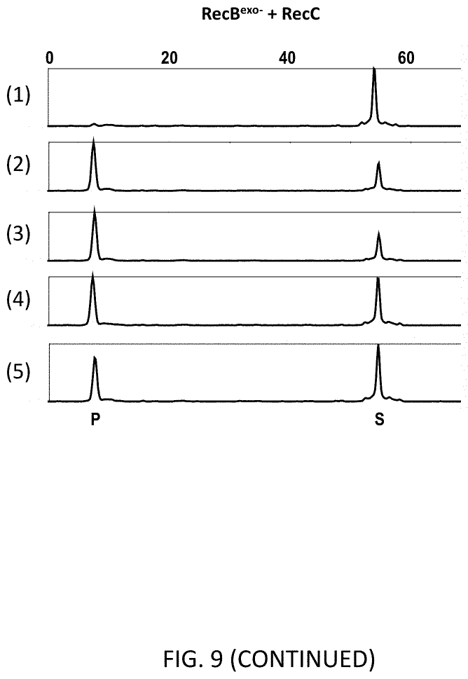

DNA unwinding activity of either RecB^{exo-} or RecB^{exo-}C DNA helicases was analyzed using the unwinding assay described in EXAMPLE 8 and shown in FIG. 4A. The obtained results confirmed that RecB^{exo-} alone is a weak DNA helicase, but its unwinding activity significantly increases after interaction with RecC protein (FIG. 9).

### Example 16: RecB^{exo-}C DNA Helicases Assist CbAgo in Cleaving Targets on Ds Substrates

A 322 bp long ds DNA fragment labeled with a 5′-FAM on one strand and a 5′-ROX on the opposite strand was amplified by PCR using a 3536-3858 nt locus of φX174 phage DNA as a template. The generated substrate in the middle encompassed a 53 nt sequence segment which was used to characterize the effect of guide sequence on the CbAgo cleavage of single-stranded DNA (FIG. 3A). Therefore, the same array of 13 guides could be used to investigate the cleavage of the respective targets but now imbedded within a 322 bp ds DNA (FIG. 10A). CbAgo was loaded with each individual guide and CbAgo/guide complex activity was monitored over time in the presence of 0.25 μM RecB^{exo-}C helicase as described in EXAMPLE 7. Results presented in FIG. 10B clearly indicate that CbAgo is capable of cleaving targets imbedded within ds DNA substrate when reaction is supplemented with RecB^{exo-} C helicase. Overall, CbAgo loaded with majority of the tested guides cleaved the respective targets up to 80-100% in 16 minutes indicating that CbAgo cleavage rates on 322 bp DNA were similar to those observed on 53 nt ss DNA (FIG. 10B and FIG. 3B). But guides T1-C1 and T2-A1 which resulted in a low ss DNA cleavage efficiency also showed the lowest cleavage efficacy on ds DNA.

Additional guides, B1, B2, B3 and B4, were designed to target the opposite strand of 322 bp DNA fragment (FIG. 11A). CbAgo loaded with these guides showed even faster cleavage rates as 45-60% of the respective targets were cleaved in the first 2 minutes at 37° C., and a complete target cleavage was achieved after 16-minute incubation. Altogether, the obtained results presented a strong evidence that CbAgo effectively can find and cleave targets imbedded on long ds substrates after RecB^{exo-}C helicase rapidly unwinds DNA strands.

### Example 17: RecB^{exo-}C DNA Helicase Assist CbAgo in Cleaving Double-Stranded Targets on DNA Substrates

Individual DNA strands are cleaved independently by two CbAgo/guide complexes therefore, the two guides can be prearranged to produce any custom-designed cleavage products which might be tailed with either 5′- or 3′-single-stranded extensions of varying length. The concurrent cleavage of opposite DNA strands was explored using a 322 bp ds DNA fragment labeled with a 5′-FAM on one strand and a 5′-ROX on the opposite strand. Two guide pairs were initially selected to test the efficiency of double-strand cleavage by CbAgo in the presence of RecB^{exo-}C helicase. CbAgo loaded with guide pair T2+B1 was expected to produce cleavage products tailed with 5 nt-long 3′-ss extensions (FIG. 12A), whereas CbAgo loaded with guide pair T1-C2+B4 was expected to produce cleavage products tailed with 5 nt-long 5′-ss extensions (FIG. 12C). CbAgo was pre-loaded with individual guides at a 1:2 molar concentration ratio, then the two opposing CbAgo/guide complexes were combined in the same reaction with RecB^{exo-} C helicase and cleavage of each DNA strand was monitored over time. Data presented in FIG. 12B revealed that double-strand cleavage by CbAgo/T2+B1 proceeded to completion and displayed cleavage rates similar to the rates observed in single-guided reactions. In contrast, the CbAgo/T1-C2+B4 cleavage was significantly slower and both strands remained only partially cleaved even after 64 minutes (FIG. 12D). Each DNA strand was cleaved to completion if CbAgo/T1-C2 and CbAgo/B4 complexes were used in separate reactions to cleave ds DNA (FIG. 10B and FIG. 11B). One-strand cleavage results allowed to assume that decline in double-strand cleavage most likely was triggered by the arrangement of T1-C2+B4 guide pair on ds target that was aimed to produce cleavage products tailed with 5 nt-long 5′-ss extensions.

To further explore the observed bias, CbAgo cleavage of 322 bp 5′FAM/ROX-labeled DNA was compared in a series of experiments using a set of previously designed thirteen guides which were programmed to cleave the 5′FAM-labeled DNA strand at positions shifted by one nucleotide along substrate sequence. For cleavage of the 5′ROX-labeled strand either guide B1 or guide B4 was used. In one series of experiments, the use of 13 guides in combination with the guide B1 allowed to generate CbAgo cleavage products tailed with 3′-single-stranded extensions varying from 1 nt up to 13 nt in length (FIG. 13A). In another series of experiments, the thirteen guides were combined with guide B4 to generate CbAgo cleavage products tailed with 5′-single-stranded extensions varying from 2 nt up to 14 nt in length (FIG. 14A). CbAgo rapidly cleaved DNA targets when loaded with guide pairs which had been programmed to produce 3′-staggered cuts varying from 1 nt to 13 nt in length (FIG. 13B). However, if the guides were programmed to produce 5′-staggered cuts, CbAgo cleavage efficiency was reduced resulting in only partial target cleavage after 64-minute incubation (FIG. 14B). The largest decline was observed with guide pairs which were positioned to produce 5′-staggered double-stranded breaks varying from 5 nt to 8 nt in length, although CbAgo activity partially recovered when the 5′-staggered double-stranded break was increased from 9 nt up to 13 nt in length (FIG. 14B). Under reaction conditions here, CbAgo was loaded with each guide at a 1:2 molar concentration ratio to abolish a non-specific DNA “chopping” by a guide-free CbAgo. When two CbAgo/guide complexes are combined, the presence of free guides potentially might interfere with ds DNA cleavage by CbAgo. To verify this concept, CbAgo/guide complexes were formed using a 1:1 molar concentration ratio, and ds target cleavage was evaluated again with guide pairs which produced cleavage products tailed with 5′-single-stranded extensions (FIG. 14A). The obtained results indicated that CbAgo cleavage efficiency significantly increased when CbAgo was loaded with guides at the 1:1 molar concentration ratio (FIG. 14C). The detailed analysis of varying CbAgo:guide molar concentration ratios was performed using a T1-A1+B4 guide pair. Reduced double-strand cleavage by CbAgo/T1-A1+B4 was observed when guide concentration was 21.6-fold higher than CbAgo concentration. However, CbAgo activity gradually increased when Ago:guide molar concentration ratio was less than 1:1.4, proceeding to complete cleavage of both DNA strands at the 1:1 Ago:guide molar concentration ratio (FIG. 15). Taken together the results obtained indicate that CbAgo cleavage activity might be inhibited by the presence of free guides in the reaction, but mainly if guides are designed to yield cleavage products tailed with 5′-single-stranded extensions. In contrast, no double-strand cleavage inhibition occurs when a 2-fold molar excess of guides over CbAgo is used to form CbAgo/guide complexes which produce 3′-staggered breaks of varying length.

### Example 18: CbAgo Acts as Programmable DNA-Guided Endonuclease on Linear Double-Stranded DNA

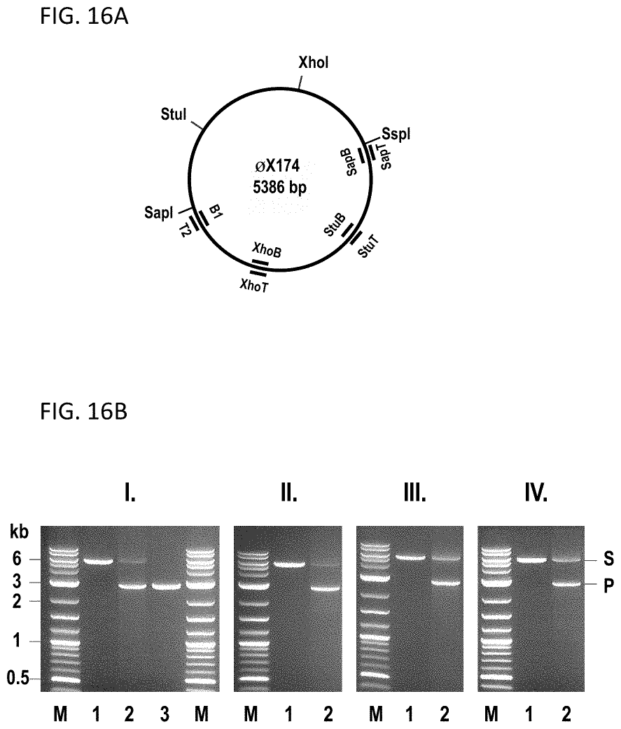

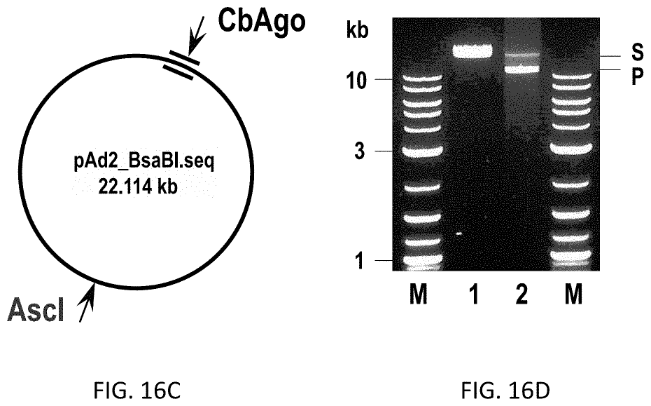

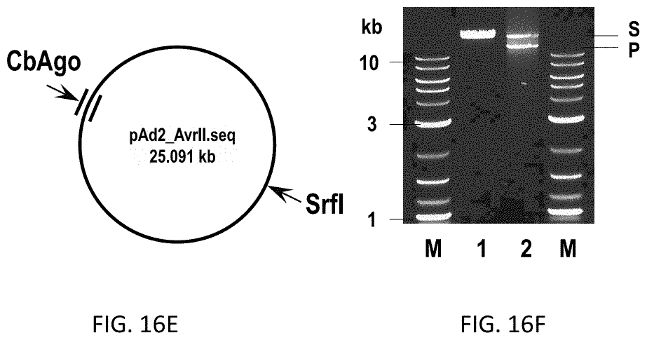

The linearized φX174 phage DNA was used to explore if CbAgo can rapidly locate and cleave targets on substantially longer ds DNA during strand unwinding progression catalyzed by RecB^{exo-}C helicase. The schematic overview of employed guide pairs on the circular φX174 DNA is shown in FIG. 16A. On SspI-linearized φX174 DNA the CbAgo/T2+B1 was programmed to generate two identical size DNA fragments (2.69 kb each) that could be detected by agarose gel-electrophoresis (FIG. 16B, PANEL I). The obtained results show that as soon as RecB^{exo-} C unwinds 5.386 kb DNA, the two CbAgo/guide complexes find the respective complementary targets and efficiently cleave both DNA strands (FIG. 16B, LANE 2 in PANEL I). The CbAgo cleavage is directed by the guide as the size of cleavage products is identical to the size of fragments generated by SapI restriction endonuclease (FIG. 16B, LANE 3 in PANEL I). Three additional locations of φX174 DNA were targeted with CbAgo programmed with different guide pairs (FIG. 16B, PANELS II-IV). All targeted locations were specifically cleaved by CbAgo, however some differences in cleavage efficiency were observed. Two sites targeted by CbAgo programmed with either guides T2+B1 or guides XhoT+XhoB were cleaved close to completion (FIG. 16B, PANELS I and II). But CbAgo cleaved slightly less efficient at the other two sites targeted with guide pairs SapT+SapB or StuT+StuB as in both cases the 60-minute incubation yielded incomplete cleavage of substrate DNA (FIG. 16B, PANELS III and IV). Guide pairs were designed to generate cleavage products with 5 nt-long 3′-ss extensions at the cut site. The observed differences in ds DNA cleavage most likely are due to the bias for certain nucleotide sequence within the guides, the phenomenon previously observed on a shorter substrate with different guide pairs (FIG. 10B).

Cleavage of longer than 5-6 kb DNA at 37° C. was tested with a CbAgo/RecB^{exo-}C combination. Two plasmids, pAd2_BsaBI (22.114 kB in length) and pAd2_AvrII (25.091 kB in length) were linearized with AscI and SrfI, respectively, and two guide pairs were designed to target each DNA at the midpoint (FIG. 16C and FIG. 16E). In the presence of RecB^{exo-}C, CbAgo programmed with the respective guide pair specifically cleaved targeted sites as indicated by the appearance of 11 kb and 12.5 kb cleavage products (LANE 2 in FIG. 16D and FIG. 16F). The results confirmed that RecB^{exo-}C is a processive and fast DNA helicase. But the most exciting outcome is that CbAgo can rapidly locate and cleave complementary targets on long ds DNAs at physiological temperature before strand reannealing takes place.

### Example 19: PCR-Free Method for Seamless Assembly of DNA Fragments by Using CbAgo and RecB^{exo-}C

A method allowing seamless assembly of CbAgo-cleaved DNA fragments was developed (FIG. 17A). If CbAgo targets are selected close to DNA end, the CbAgo/RecB^{exo-}C cleavage can result in generating long ds DNA flanked with single-stranded termini of any preferred length and composition. But ds cleavage close to the end also removes from DNA short terminal fragments, thus resulting in a loss of sequence information. To restore the removed nucleotide sequence, two complementary single-stranded oligonucleotides may be synthesized. The sequence of each oligonucleotide consists of the consecutively linked sequences that are identical either to the top strands or to the bottom strands on the removed DNA fragments. The complementary oligonucleotides are then annealed to form a double-stranded “Bridge” oligonucleotide, which on both ends is flanked by single-stranded extensions. When combined, two DNA fragments and Bridge oligonucleotide can directionally assemble into a recombinant molecule via complementary single-stranded extensions (FIG. 17A).

Assembly of 332 bp and 300 bp 5′-FAM/ROX labeled PCR fragments (referred to as DNA1 and DNA2, respectively) was evaluated. On DNA1, the CbAgo target site was selected close to the 5′-ROX labeled end, whereas on DNA2 the CbAgo target site was selected close to the 5′-FAM-labeled end. The guide pairs T1+B2 and T5+B5 complemented DNA1 and DNA2, respectively, and both guide pairs were arranged to create 8 nt-long 3′-ss extensions on CbAgo cleavage products. In this arrangement, the CbAgo cleavage of either DNA produced throwaway terminal fragments, a 5′-ROX labeled 15 bp fragment and a 5′-FAM labeled 14 bp fragment, which were eliminated by column purification (FIG. 17B). DNA1 and a synthetic 29 bp-long Bridge oligonucleotide (0.3 pmols each) were then ligated for 15 minutes at 37° C. to generate a 346 bp intermediate ligation product. The ligation reaction was then supplemented with 0.3 pmols of DNA2 and ligation continued for another 15 minutes. Ligation results presented in FIG. 17B, revealed the appearance of 346 bp 5′-FAM labeled ligation product during the 1^{st} ligation step, and the appearance of 632 bp 5′-FAM/ROX labeled ligation product during the 2^{nd} ligation step indicating that both CbAgo cleavage products and Bridge oligonucleotide were directionally assembled into a recombinant DNA. Taken together the obtained results provided a strong evidence that CbAgo can generate sequence-specific single-stranded extensions that are ready for ligation without any further enzymatic treatment, thus suggesting that PCR-free seamless assembly of natural DNA molecules can be accomplished by using CbAgo/RecB^{exo-}C programmable DNA endonuclease.

### Example 20: RecΔBC DNA Helicase Assist CbAgo in Cleaving Double-Stranded Targets on DNA Substrates

RecB is organized into a 100-kDa N-terminal helicase domain and 30-kDa C-terminal exonuclease/endonuclease domain. The C-terminal domain functions independently as an endo- and exonuclease and is responsible for all nuclease activities associated with the RecBCD. A truncated RecB variant comprising a helicase domain (RecB₁₋₉₂₉) has ATP-dependent DNA unwinding activity, but no longer exhibits nuclease activity. Based on the above information, a truncated RecB₁₋₉₂₉ C helicase that lacks nuclease activity was tested for ability to help CbAgo in cleaving double-stranded targets at 37° C.

RecB₁₋₉₂₉ variant, referred here as RecΔB, was created as described in EXAMPLE 5. The purified RecΔB subunit was mixed with RecC subunit at 1:1 stoichiometry to the final 10 μM concentration to reconstitute RecΔBC helicase. RecΔBC was evaluated as a CbAgo partner in the cleavage of 80 bp blunt-ended DNA substrate at 37° C. In the presence of RecΔBC DNA helicase, only 25% of substrate was cleaved by CbAgo/guide B12 complex in the first 15 minutes (FIG. 18A, LANE 3). The CbAgo cleavage efficiency was increased by 2-fold when the reaction containing RecΔBC was supplemented with CbuRecQ DNA helicase (FIG. 18A, LANE 4). Addition of the third DNA helicase to the same reaction, Slur07Dda, did not help to further increase CbAgo cleavage efficiency (FIG. 18A, LANE 5).

CbAgo cleavage of 239 bp and 619 bp ds DNA was evaluated either in the presence of RecΔBC alone or in the presence of two helicase mixtures, RecΔBC+CpeRecQ and RecΔBC+CbuRecQ. 239 bp and 619 bp FAM-labeled DNA substrates were generated by PCR amplification using phage φX174 DNA as a template as described in EXAMPLE 7. For single-strand cleavage experiments, CbAgo was loaded with a guide T2 during a 15-minute incubation at 37° C. The CbAgo cleavage reaction was assembled in a 20 μl of 1× CutSmart buffer containing 50 nM DNA substrate, CbAgo/T2 at a 0.25/0.25 μM final concentration, 0.25 μM RecΔBC and 5 mM ATP. When applied, either CpeRecQ or CbuRecQ was added to the reaction at a final 5.5 μM or 6.5 μM concentration, respectively, and the reactions were incubated for 1 hour at 37° C. Single-strand cleavage results presented in FIGS. 18B and 18C revealed that only RecΔBC+CpeRecQ helicase mixture efficiently stimulated CbAgo/T2 cleavage of ds DNA, however, the cleavage efficiency declined when the length of DNA substrate was increased from 239 bp to 619 bp. For double-stranded cleavage, CbAgo was programmed with guides T2 and B1 in two separate reactions and then both CbAgo/guide complexes were combined with RecΔBC+CpeRecQ as described above. Results presented in FIG. 18D revealed that up to 80% of either 239 bp or 619 bp substrate was cleaved on both DNA strands.

In summary, the results indicated that RecΔBC DNA helicase did not support CbAgo programmable cleavage of ds DNA under conditions tested. The unwinding activity of RecΔBC was increased only in the presence of a high concentration of CpeRecQ DNA helicase. For a rapid and processive DNA unwinding activity, RecB subunit must form a complex with RecC subunit. Most likely, deletion of 30 kD C-terminal nuclease domain destabilized interactions between the RecΔB and RecC subunits, thus making a structurally unstable RecΔBC complex and causing RecΔB helicase to act alone. Reconstitution of RecB₁₋₉₂₉ with RecC and RecD subunits leads to processive unwinding of a 4.3 kb linearized ds DNA plasmid. Possibly, the RecD subunit can be used to form a stable RecΔBCD complex and thus, to increase DNA unwinding activity to levels that allow efficient cleavage of long ds DNAs by CbAgo. Also, data presented in EXAMPLE 15 shows that full-length RecB^{exo-}C variant carrying a catalytically inactive C-terminal nuclease domain exhibits vary rapid and processive DNA unwinding activity that is sufficient for CbAgo cleavage of 22-25 kb long double-stranded DNAs. Potentially, a RecB variant with a partially deleted nuclease domain can be created which can form a stable complex with RecC subunit, but no longer exhibits nuclease activity.

### Example 21: CbAgo/RecB^{exo-}C Programmable Endonuclease is Active at 25-45° C.

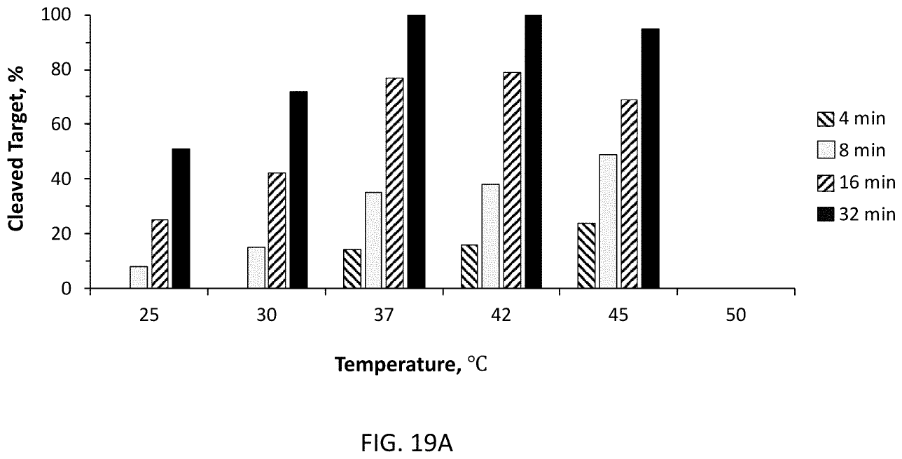

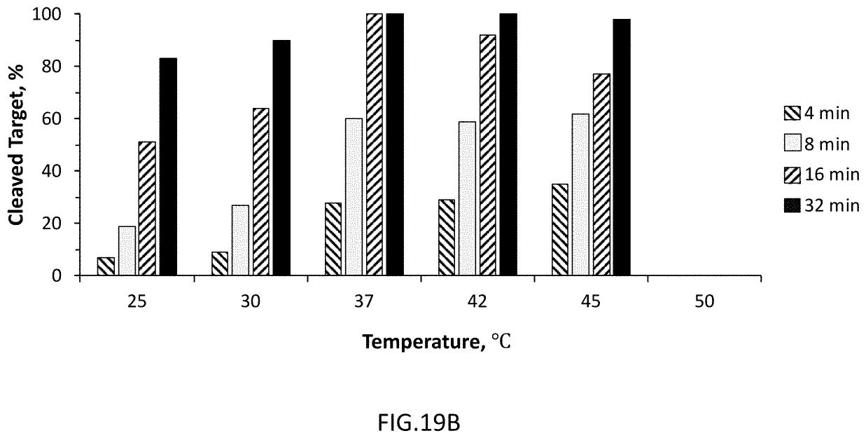

CbAgo is capable of cleaving ss DNA at wide range of temperatures spanning from 30° C. up to 75° C. However, E. coli RecBCD enzyme was shown to unwind DNA duplex at 20-37° C. temperature range. To determine the optimal temperature range for CbAgo/RecB^{exo-}C programmable endonuclease, double-strand DNA cleavage reactions were carried out at 25, 30, 37, 42, 45 and 50° C. temperatures. 322 bp FAM/ROX-labeled DNA substrate was cleaved simultaneously on both strands with CbAgo loaded with guides T2 and B1 in the presence of RecB^{exo-}C DNA helicase as described in EXAMPLE 7. The cleavage efficiency of each DNA strand was evaluated after incubation for 4, 8, 16 and 32 minutes at the indicated temperature. The results presented in FIG. 19A show cleavage of 5′-FAM labeled DNA strand with CbAgo loaded with guide T2. The results presented in FIG. 19B show cleavage of 5′-ROX labeled strand with CbAgo loaded with guide B1. Under the conditions tested, CbAgo/RecB^{exo-}C programmable endonuclease exhibits the highest activity at 37-42° C. and lower activity at 25-30° C. During the initial reaction time, CbAgo/RecB^{exo-}C cleaves DNA substrate faster at 45° C. than at 42-37° C. but at later timepoints the cleavage slows down. Without limiting any embodiment to any particular mechanism of action, this may be due to a thermal inactivation of RecB^{exo-}C DNA helicase over time at higher than 42° C. temperature. At 50° C. temperature, CbAgo/RecB^{exo-}C showed no substrate cleavage activity under these conditions.

### Example 22: Comparison of CbAgo/RecB^{exo-}C Cleavage Activity on Unmodified, 5mC-Modified, 5hmC-Modified and 5ghmC-Modified DNA Substrates

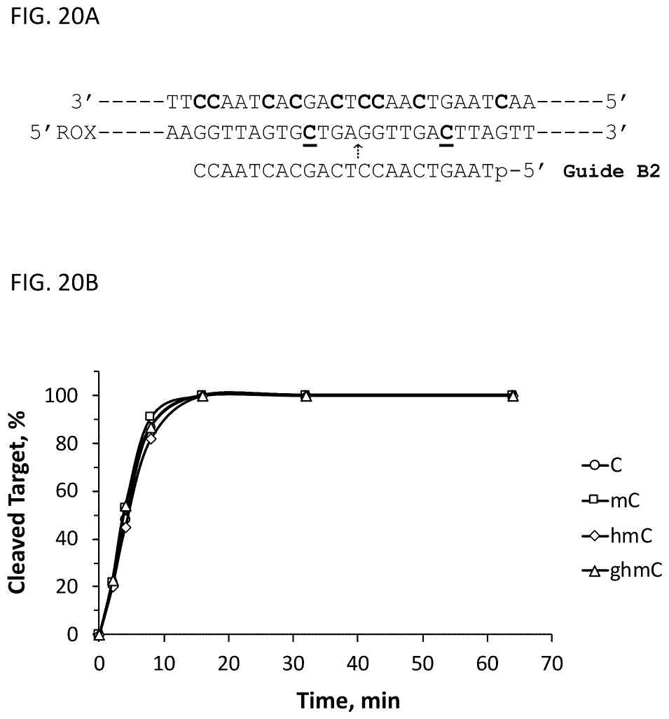

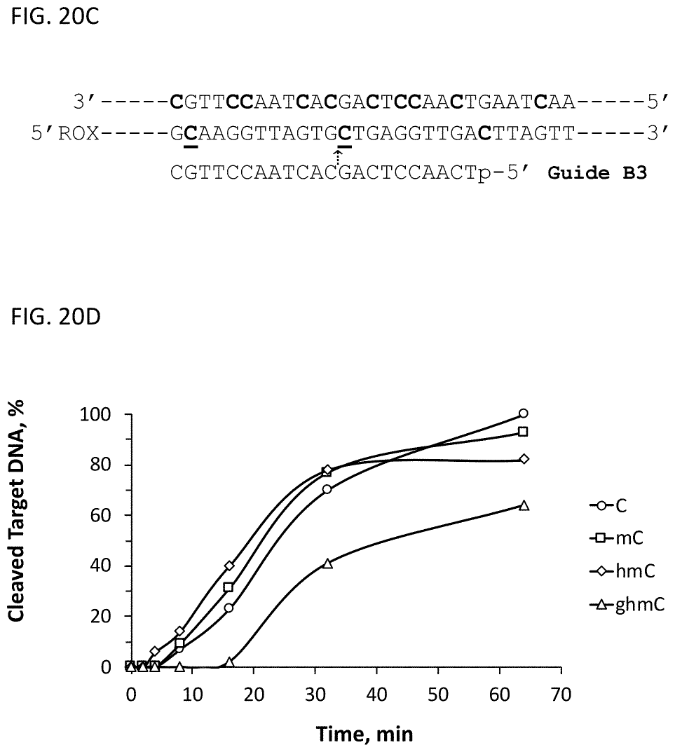

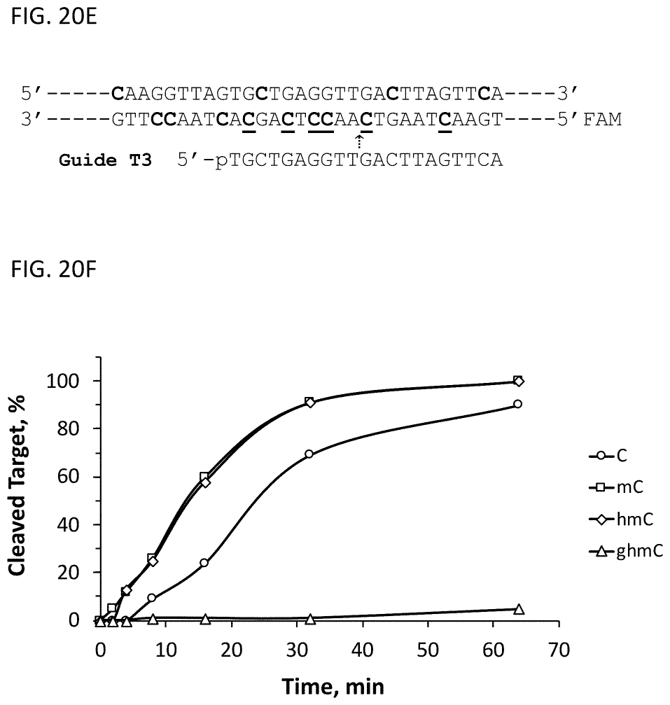

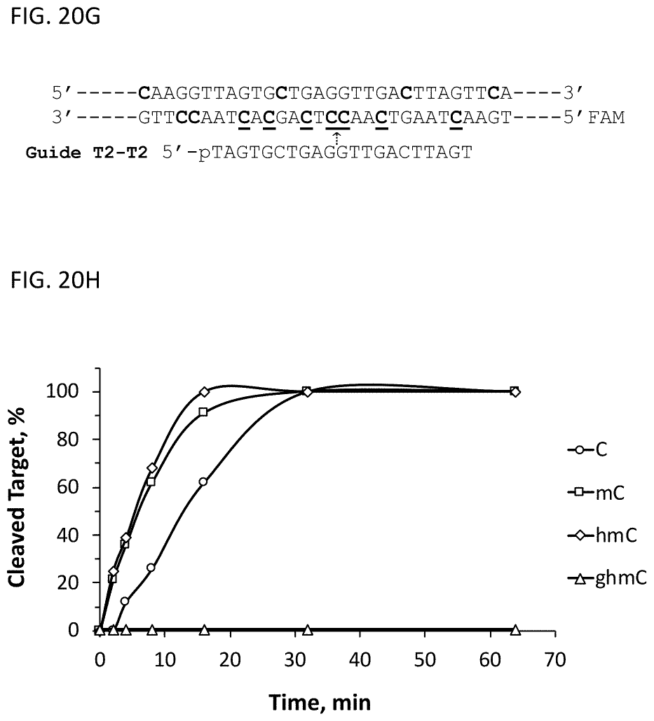

Modified 322 bp DNA substrates were generated by PCR as described in EXAMPLE 7. DNA amplification with dNTPs including 5-methyl-dCTP or 5-hydroxymethyl-dCTP allowed 5-methylcytosine-containing substrates (mC) or hydroxymethylcytosine containing substrates (hmC), respectively, to be generated. To generate a beta-glucosyl-5-hydroxymethylcytosine containing substrate (ghmC), the hmC substrate was glucosylated using T4-BGT glucosyltransferase in the presence of UDP-Glucose. Target DNA sequence complementary to B2 guide carried two cytosine residues which were subjected to a modification, however the cytosines were positioned at either 3-nt or 6-nt distance from the CbAgo cleavage site (FIG. 20A). Time course cleavage results showed that regardless of the cytosine modification CbAgo/B2 cleaved modified substrates with the same efficiency as the substrate containing unmodified cytosine (FIG. 20B). DNA sequence targeted by CbAgo/B3 also contained two cytosine residues which were subjected to the modification. One modified cytosine was located far-off the cleavage site, but another cytosine was positioned at a 10^{th} position, if counting nucleotides from a 5′-phosphate of B3 guide, indicating that a cleavable phosphodiester bond was situated on the 5′-side to the glucosylated cytosine (FIG. 20C). The results showed that CbAgo/B3 cleaved DNA targets containing either C, mC or hmC residues with a similar efficiency. However, a reduced cleavage efficiency was observed on ghmC-containing target (FIG. 20D) suggesting that cytosine glucosylation might inhibit CbAgo cleavage when glucosylated cytosine is located in close proximity to the cleavage site. To further explore the effect of DNA glucosylation, a 5′-FAM labeled strand of 322 bp DNA was targeted with either CbAgo/T3 (FIG. 20E) or CbAgo/T2-T2 (FIG. 20G). CbAgo/T3 targeted sequence contained a modified cytosine positioned at a 11^{th} position, if counting nucleotides from a 5′-phosphate of T3 guide, indicating that CbAgo was expected to cleave a phosphodiester bond situated on the 3′-side to the glucosylated cytosine (FIG. 20E). CbAgo/T2-T2 targeted sequence contained two modified cytosines located at the 10^{th} and 11^{th} positions, if counting nucleotides from a 5′-phosphate of T2-T2 guide, indicating that CbAgo cleavage site on both sides was flanked by glucosylated cytosines (FIG. 20G). Either 5mC- or 5hmC-containing substrate was efficiently cleaved by both CbAgo/T3 (FIG. 20F) and CbAgo/T2-T2 (FIG. 20H). Under the conditions tested, cleavage of the glucosylated DNA substrate was detected after 64-minute incubation with CbAgo/T3 (FIG. 20F) and no cleavage was detected when glucosylated substrate was targeted with CBbAgo/T2-T2 (FIG. 20H). In either case, the targeted nucleotide sequence carried additional modified cytosines and, without limiting any embodiment to any particular mechanism of action, their glucosylation may have contributed to the observed reduction in CbAgo cleavage. Altogether, the obtained results indicate that CbAgo activity is impaired on glucosylated DNA, especially when glucosylated cytosine residues are situated in the vicinity of the phosphodiester bond targeted for cleavage.

### Example 23: CbAgo Cleaves Double-Stranded Targets after Denaturation of DNA Duplex by Treatment with Formamide at High Temperature

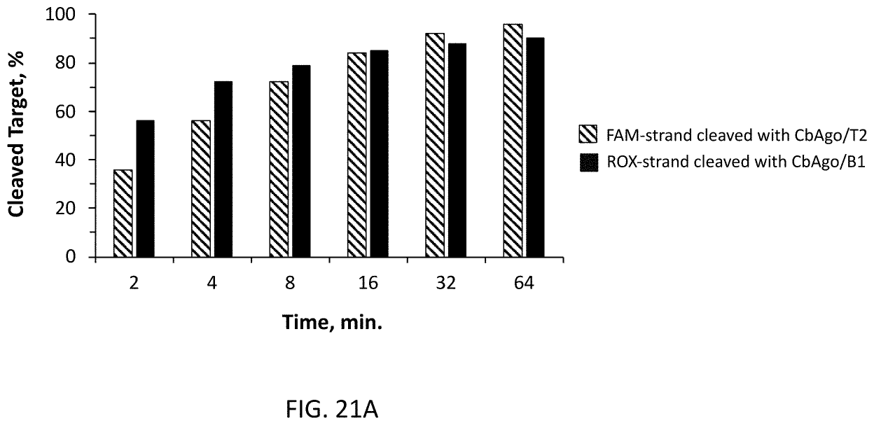

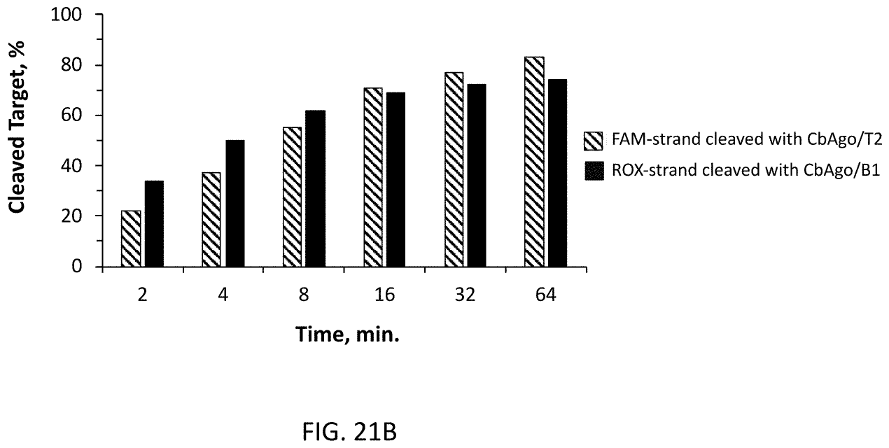

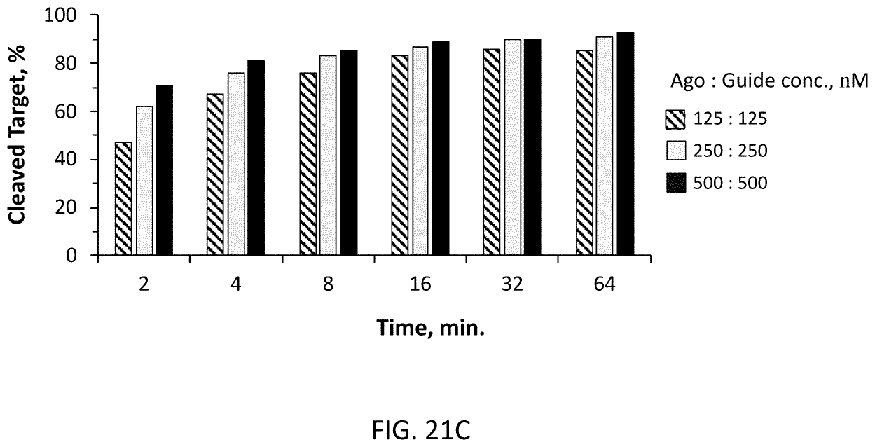

CbAgo functions at a range of temperatures (e.g., 30-75° C.) which do not (alone) facilitate separation of ds DNA into single strands. In this example, ds DNA was subjected to physical denaturation (e.g., high temperature) in the presence of chemical denaturant (e.g., formamide) to generate single-stranded targets for CbAgo cleavage. To generate denatured DNA substrate, 2.5 pmol/5 μl of either 322 bp or 619 bp DNA was combined with 2 μl 100% formamide in a 10 μl volume and incubated for 10 minutes at 85° C., then quickly placed in ice/ethanol bath for 1 minute. CbAgo was pre-loaded with guides T2 and B1 at a 1:1 molar concentration ratio in two separate 15 μl reactions each containing 1.5 μl 10× CutSmart buffer, 1.5 μl 5 μM guide, 1.5 μl 5 μM CbAgo. Two CbAgo/guide complexes, 12.5 μl each, were combined with 15 μl 1× CutSmart buffer and a cleavage reaction was initiated at 37° C. by addition of 10 μl denatured DNA substrate (50 nM final concentration). Formamide concentration in CbAgo cleavage reaction was maintained at 4% final concentration to slow down DNA strand reannealing at 37° C. 5 μl samples were removed from the reaction at indicated time points and quenched with 50 mM EDTA. DNA samples were analyzed by capillary electrophoreses as described in EXAMPLE 6. Data presented in FIG. 21A an FIG. 21B revealed that both strands of either 322 bp or 619 bp DNA were efficiently cleaved by CbAgo indicating that the physical/chemical DNA denaturation method can be employed to separate ds DNA into single strands. Over time, CbAgo cleavage gradually increased until a 16-minute time point, but then plateaued at 80-90% for 322 bp substrate and at 70-80% for 619 bp substrate (FIG. 21A and FIG. 21B, respectively). The DNA substrate may have been partially resistant to CbAgo cleavage if some remained in or reannealed to a double-stranded form. The impact of reannealing may be limited by working to cleave the DNA target as soon as possible (prior to strand reannealing).

CbAgo cleavage efficiency of formamide-denatured, 616 bp ROX-labeled DNA was compared using increasing CbAgo/guide concentrations. CbAgo/T2 and CbAgo/B1 complexes at either 125:125 nM, 250:250 nM or 500:500 nM Ago:guide concentration, were each combined with 50 nM formamide-denatured, 616 bp ROX-labeled DNA and cleavage was monitored over time. The percentage of cleaved DNA at a 2-minute time point was 1.5-fold more for reactions with 500 nM CbAgo than for reactions with 125 nM CbAgo (FIG. 21C). At a 10:10:1 molar concentration ratio of Ago:guide:target, about 80% of DNA was cleaved after 4-minutes from the start of the reaction. But at later time points the CbAgo cleavage slowed down, and in total only 93% of DNA was cleaved after 64-minute incubation. The obtained results confirmed that the use of high CbAgo concentration is beneficial in achieving efficient DNA cleavage in the early stage of the reaction when DNA is present in a single-stranded form. But results also showed that formamide denaturation did not yield complete strand separations under the conditions tested, which may be due to the presence of some (5-10%) substrate DNA in double stranded form.

What is claimed is:

1. A non-naturally occurring composition comprising a helicase, a first mesophilic Argonaute bound to a first guide, and optionally, a second mesophilic Argonaute bound to a second guide.

2. A composition according to claim 1, wherein the first Argonaute and the first guide are present at an Argonaute:guide molar concentration ratio of 2:1 to 1:2.

3. A composition according to claim 1, wherein the first Argonaute and the first guide are present at an Argonaute:guide molar concentration ratio equal to or lower than 1:1.4.

4. A composition according to claim 1, wherein the second Argonaute and the second guide are present at an Argonaute:guide molar concentration ratio of 2:1 to 1:2.

5. A composition according to claim 1, wherein the second Argonaute and the second guide are present at an Argonaute:guide molar concentration ratio equal to or lower than 1:1.4.

6. A composition according to claim 1 further comprising a double-stranded polynucleotide.

7. A composition according to claim 6, wherein the polynucleotide comprises on a first strand a sequence complementary to the first guide.

8. A composition according to claim 7, wherein the polynucleotide comprises on a second strand a sequence complementary to the second guide.

9. A composition according to claim 1, wherein
(a) the Argonaute bound to the first guide is selected from an Aquifex aeolicus Argonaute, an Aquifex aeolicus Argonaute, a Microsystis aeruginosa Argonaute, a Clostridium bartlettii Argonaute, an Exiguobacterium Argonaute, an Anoxybacillus flavithermus Argonaute, a Halogeometricum borinquense Argonaute, a Halorubrum lacusprofundi Argonaute, an Aromatoleum aromaticum Argonaute, a Synechococcus Argonaute, a Clostridium butyricum Argonaute (CbAgo), a Clostridium disporicum Argonaute (CdAgo), a Clostridium perfringens Argonaute (CpAgo), a Clostridium sartagoforme Argonaute (CsAgo), a Clostridium saudiense Argonaute (CaAgo), an Intestinibacter bartlettii Argonaute (IbAgo) and, in each case, homologues having at least 90% amino acid sequence identity thereto; and
(b) the Argonaute bound to the second guide is independently selected from an Aquifex aeolicus Argonaute, an Aquifex aeolicus Argonaute, a Microsystis aeruginosa Argonaute, a Clostridium bartlettii Argonaute, an Exiguobacterium Argonaute, an Anoxybacillus flavithermus Argonaute, a Halogeometricum borinquense Argonaute, a Halorubrum lacusprofundi Argonaute, an Aromatoleum aromaticum Argonaute, a Synechococcus Argonaute, a Clostridium butyricum Argonaute (CbAgo), a Clostridium disporicum Argonaute (CdAgo), a Clostridium perfringens Argonaute (CpAgo), a Clostridium sartagoforme Argonaute (CsAgo), a Clostridium saudiense Argonaute (CaAgo), an Intestinibacter bartlettii Argonaute (IbAgo) and, in each case, homologues having at least 90% amino acid sequence identity thereto.

10. A composition according to claim 1, wherein the Argonaute bound to the first guide is CaAgo, CbAgo, CdAgo, CpAgo, CsAgo or IbAgo and the Argonaute bound to the second guide is independently CaAgo, CbAgo, CdAgo, CpAgo, CsAgo or IbAgo, and, in each case, homologues having at least 90% amino acid sequence identity thereto.

11. A composition according to claim 1, wherein the helicase is selected from the group consisting of an EcoRecQ DNA helicase from Escherichia coli, a CpeRecQ from Clostridium perfringens, a Cbu RecQ from Clostridium butyricum, a DNA helicase from T4-like bacteriophage, a T7 bacteriophage gp4 DNA helicase, RecBCD-family helicases from Escherichia coli, a modified RecBCD helicase, a UvrD/PcrA family helicase, an E. coli Rep, an M. tuberculosis PcrA, an M. leprae PcrA, and an Escherichia coli Tra helicase.

12. A composition according to claim 1, wherein the first guide and the second guide are independently 12-60 nucleotides in length.

13. A method of forming a double strand break in a double-stranded polynucleotide at a target position in the polynucleotide, the method comprising:
contacting
(a) a double-stranded polynucleotide having a first target sequence on a first strand of the polynucleotide and a second target sequence on the opposite strand,
(b) a helicase,
(c) an Argonaute with a first bound guide having a sequence complimentary to the first target sequence, and
(d) an Argonaute with a second bound guide having a sequence complimentary to the second target sequence under conditions that permit hybridization of complimentary sequences and cleavage of the first strand by the (c) Argonaute and cleavage of the second strand by the (d) Argonaute to produce a double strand break in the polynucleotide.

14. A method according to claim 13, wherein the contacting further comprises contacting at a temperature of 25° C. to 45° C.

15. A method according to claim 13, wherein the double strand break in the polynucleotide forms at least a first fragment of the polynucleotide and a second fragment of the polynucleotide.

16. A method according to claim 13, wherein the first guide has a sequence complementary to the first target sequence.

17. A method according to claim 13, wherein the double strand break forms blunt ends.

18. A method according to claim 13, wherein the double strand break forms an overhang from 1 to 50 nucleotides in length.

19. A method according to claim 13, wherein the double strand break forms an overhang from 51 to 100 nucleotides in length.

20. A method for cleaving a double strand nucleic acid substrate at a target sequence, comprising:
(a) contacting a helicase, an Argonaute, a guide DNA bound to the Argonaute, and a polynucleotide comprising a target sequence that is complementary to at least part of the guide DNA, to produce a reaction mix; and
(b) incubating the reaction mix at a temperature of 25° C. to 45° C.,
wherein the nucleic acid is cleaved.

21. A method according to claim 20, wherein the contacting further comprises contacting the helicase, the Argonaute, the guide DNA bound to the Argonaute, the polynucleotide, a second Argonaute, and a second guide bound to the second Argonaute, wherein the polynucleotide further comprises a second target sequence that is complementary to at least part of the second guide DNA.


SEQUENCE LISTING (94 sequences)

SEQ ID NO: 1 - protein, 929 aa, Artificial Sequence
  Synthetic construct
  MISC_FEATURE: RecB protein sequence (truncated RecB with deleted nuclease domain)
MSDVAETLDPLRLPLQGERLIEASAGTGKTFTIAALYLRLLLGLGGSAAFPRPLTVEELLVVTFTEAATAELRGRIRSNIHELRIACLRETTDNPLYERLLEEIDDKAQAAQWLLLAERQMDEAAVFTIHGFCQRMLNLNAFESGMLFEQQLIEDESLLRYQACADFWRRHCYPLPREIAQVVFETWKGPQALLRDINRYLQGEAPVIKAPPPDDETLASRHAQIVARIDTVKQQWRDAVGELDALIESSGIDRRKFNRSNQAKWIDKISAWAEEETNSYQLPESLEKFSQRFLEDRTKAGGETPRHPLFEAIDQLLAEPLSIRDLVITRALAEIRETVAREKRRRGELGFDDMLSRLDSALRSESGEVLAAAIRTRFPVAMIDEFQDTDPQQYRIFRRIWHHQPETALLLIGDPKQAIYAFRGADIFTYMKARSEVHAHYTLDTNWRSAPGMVNSVNKLFSQTDDAFMFREIPFIPVKSAGKNQALRFVFKGETQPAMKMWLMEGESCGVGDYQSTMAQVCAAQIRDWLQAGQRGEALLMNGDDARPVRASDISVLVRSRQEAAQVRDALTLLEIPSVYLSNRDSVFETLEAQEMLWLLQAVMTPERENTLRSALATSMMGLNALDIETLNNDEHAWDVVVEEFDGYRQIWRKRGVMPMLRALMSARNIAENLLATAGGERRLTDILHISELLQEAGTQLESEHALVRWLSQHILEPDSNASSQQMRLESDKHLVQIVTIHKSKGLEYPLVWLPFITNFRVQEQAFYHDRHSFEAVLDLNAAPESVDLAEAERLAEDLRLLYVALTRSVWHCSLGVAPLVRRRGDKKGDTDVHQSALGRLLQKGEPQDAAGLRTCIEALCDDDIAWQTAQTGDNQPWQVNDVSTAELNAKTLQRLPGDNWRVTSYSGLQQRGHGIAQDLMPRLDVDAA

SEQ ID NO: 2 - protein, 1180 aa, Artificial Sequence
  Synthetic construct
  MISC_FEATURE: RecB exo- mutant protein sequence (mutant carries mutations E1020A, D1080A and K1082A to abolish nuclease activity)
MSDVAETLDPLRLPLQGERLIEASAGTGKTFTIAALYLRLLLGLGGSAAFPRPLTVEELLVVTFTEAATAELRGRIRSNIHELRIACLRETTDNPLYERLLEEIDDKAQAAQWLLLAERQMDEAAVFTIHGFCQRMLNLNAFESGMLFEQQLIEDESLLRYQACADFWRRHCYPLPREIAQVVFETWKGPQALLRDINRYLQGEAPVIKAPPPDDETLASRHAQIVARIDTVKQQWRDAVGELDALIESSGIDRRKFNRSNQAKWIDKISAWAEEETNSYQLPESLEKFSQRFLEDRTKAGGETPRHPLFEAIDQLLAEPLSIRDLVITRALAEIRETVAREKRRRGELGFDDMLSRLDSALRSESGEVLAAAIRTRFPVAMIDEFQDTDPQQYRIFRRIWHHQPETALLLIGDPKQAIYAFRGADIFTYMKARSEVHAHYTLDTNWRSAPGMVNSVNKLFSQTDDAFMFREIPFIPVKSAGKNQALRFVFKGETQPAMKMWLMEGESCGVGDYQSTMAQVCAAQIRDWLQAGQRGEALLMNGDDARPVRASDISVLVRSRQEAAQVRDALTLLEIPSVYLSNRDSVFETLEAQEMLWLLQAVMTPERENTLRSALATSMMGLNALDIETLNNDEHAWDVVVEEFDGYRQIWRKRGVMPMLRALMSARNIAENLLATAGGERRLTDILHISELLQEAGTQLESEHALVRWLSQHILEPDSNASSQQMRLESDKHLVQIVTIHKSKGLEYPLVWLPFITNFRVQEQAFYHDRHSFEAVLDLNAAPESVDLAEAERLAEDLRLLYVALTRSVWHCSLGVAPLVRRRGDKKGDTDVHQSALGRLLQKGEPQDAAGLRTCIEALCDDDIAWQTAQTGDNQPWQVNDVSTAELNAKTLQRLPGDNWRVTSYSGLQQRGHGIAQDLMPRLDVDAAGVASVVEEPTLTPHQFPRGASPGTFLHSLFEDLDFTQPVDPNWVREKLELGGFESQWEPVLTEWITAVLQAPLNETGVSLSQLSARNKQVAMEFYLPISEPLIASQLDTLIRQFDPLSAGCPPLEFMQVRGMLKGFIDLVFRHEGRYYLLAYASNWLGEDSSAYTQQAMAAAMQAHRYDLQYQLYTLALHRYLRHRIADYDYEHHFGGVIYLFLRGVDKEHPQQGIYTTRPNAGLIALMDEMFAGMTLEEA

SEQ ID NO: 3 - protein, 1122 aa, Artificial Sequence
  Synthetic construct
  MISC_FEATURE: RecC protein sequence
MLRVYHSNRLDVLEALMEFIVERERLDDPFEPEMILVQSTGMAQWLQMTLSQKFGIAANIDFPLPASFIWDMFVRVLPEIPKESAFNKQSMSWKLMTLLPQLLEREDFTLLRHYLTDDSDKRKLFQLSSKAADLFDQYLVYRPDWLAQWETGHLVEGLGEAQAWQAPLWKALVEYTHQLGQPRWHRANLYQRFIETLESATTCPPGLPSRVFICGISALPPVYLQALQALGKHIEIHLLFTNPCRYYWGDIKDPAYLAKLLTRQRRHSFEDRELPLFRDSENAGQLFNSDGEQDVGNPLLASWGKLGRDYIYLLSDLESSQELDAFVDVTPDNLLHNIQSDILELENRAVAGVNIEEFSRSDNKRPLDPLDSSITFHVCHSPQREVEVLHDRLLAMLEEDPTLTPRDIIVMVADIDSYSPFIQAVFGSAPADRYLPYAISDRRARQSHPVLEAFISLLSLPDSRFVSEDVLALLDVPVLAARFDITEEGLRYLRQWVNESGIRWGIDDDNVRELELPATGQHTWRFGLTRMLLGYAMESAQGEWQSVLPYDESSGLIAELVGHLASLLMQLNIWRRGLAQERPLEEWLPVCRDMLNAFFLPDAETEAAMTLIEQQWQAIIAEGLGAQYGDAVPLSLLRDELAQRLDQERISQRFLAGPVNICTLMPMRSIPFKVVCLLGMNDGVYPRQLAPLGFDLMSQKPKRGDRSRRDDDRYLFLEALISAQQKLYISYIGRSIQDNSERFPSVLVQELIDYIGQSHYLPGDEALNCDESEARVKAHLTCLHTRMPFDPQNYQPGERQSYAREWLPAASQAGKAHSEFVQPLPFTLPETVPLETLQRFWAHPVRAFFQMRLQVNFRTEDSEIPDTEPFILEGLSRYQINQQLLNALVEQDDAERLFRRFRAAGDLPYGAFGEIFWETQCQEMQQLADRVIACRQPGQSMEIDLACNGVQITGWLPQVQPDGLLRWRPSLLSVAQGMQLWLEHLVYCASGGNGESRLFLRKDGEWRFPPLAAEQALHYLSQLIEGYREGMSAPLLVLPESGGAWLKTCYDAQNDAMLDDDSTLQKARTKFLQAYEGNMMVRGEGDDIWYQRLWRQLTPETMEAIVEQSQRFLLPLFRFNQS

SEQ ID NO: 4 - protein, 439 aa, Artificial Sequence
  Synthetic construct
  MISC_FEATURE: Phage Slur07 Dda helicase sequence (YP_009197435.1)
MTFDDLTEGQKNAFNTVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLIKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADAELAQQLLYVGVTRGRYDVFYV

SEQ ID NO: 5 - protein, 609 aa, Artificial Sequence
  Synthetic construct
  MISC_FEATURE: E.coli RecQ helicase sequence (WP_096324295.1)
MAQAEVLNLESGAKQVLQETFGYQQFRPGQEEIIDTVLSGRDCLVVMPTGGGKSLCYQIPALLLNGLTVVVSPLISLMKDQVDQLQANGVAAACLNSTQTREQQLEVMTGCRTGQIRLLYIAPERLMLDNFLEHLAHWNPVLLAVDEAHCISQWGHDFRPEYAALGQLRQRFPTLPFMALTATADDTTRQDIVRLLGLNDPLIQISSFDRPNIRYMLMEKFKPLDQLMRYVQEQRGKSGIIYCNSRAKVEDTAARLQSKGISAAAYHAGLENNVRADVQEKFQRDDLQIVVATVAFGMGINKPNVRFVVHFDIPRNIESYYQETGRAGRDGLPAEAMLFYDPADMAWLRRCLEEKPQGQLQDIERHKLNAMGAFAEAQTCRRLVLLNYFGEGRQEPCGNCDICLDPPKQYDGSTDAQIALSTIGRVNQRFGMGYVVEVIRGANNQRIRDYGHDKLKVYGMGRDKSHEHWVSVIRQLIHLGLVTQNIAQHSALQLTEAARPVLRGESSLQLAVPRIVALKPKAMQKSFGGNYDRKLFAKLRKLRKSIADESNVPPYVVFNDATLIEMAEQMPITASEMLSVNGVGMRKLERFGKPFMALIRAHVDGDDEE

SEQ ID NO: 6 - protein, 724 aa, Artificial Sequence
  Synthetic construct
  MISC_FEATURE: Clostridium perfringens Cpe RecQ DNA helicase sequence (WP_011590145.1)
MKRAKEILKSFYGYSEFRKGQEEIINEIISGNDVVAIMPTGGGKSICYQIPALILEGVTVVISPLISLMKDQVDAINSMGIESAYINSTLSNEEINNIFQSVKDGHIKILYVAPERLESMEFLNLVSNIDISQVAVDEAHCVSQWGHDFRSSYKFIGRFINLLRKRPVVSAFTATATEEVRKDIVKLLELNNPKVFVSGFDRDNLKIIIEKGVNKAHYILDYLKENKDVSGIIYCATRKEVDSLCELINSRGIGCTKYHAGLSDEERKKNQEDFVFDKVSVMVATNAFGMGIDKPNIRYVIHNNMPKNIEGYYQEIGRAGRDGEKSECILIFSPGDVQTQKYIIETGTLNPERKINELSKLQTMMDLVYSNGCYRRFILNYFGEDLEEDCHNCSNCEMEGELVDKTIDAQKVISCVYRMKRPFGIGVIIDVLRASKNKKIIELGLDQLSTYGIMKDYSKEGLKDFINTLISHRYIDYIGEYPVVRLNQSSIDILKGNEKVMFKEKIKAKRVSKNNELFDLLRSLRREIALEEGVPPYIVFGDNTLKEMSVRIPVNESQLSEISGVGEKKILKYGEQFLNVIKEYVKVNNIEVQWESGLKINKDELEEKFKKGNGKDGKKKSFEITIDMLREGDKFHTIAKERELSITTVLSHIQQYLEEGNKIDFEIDFDSLFSKREEELVLDAIDKVGYSKLKPIKENLEEDVSYEKIRFIVTKLTINNLRKE

SEQ ID NO: 7 - protein, 816 aa, Artificial Sequence
  Synthetic construct
  MISC_FEATURE: Clostridium butyricum Cbu RecQ DNA helicase sequence (WP_003411240.1)
MINKSPQSVLEKYYGYKSFREGQENIINSIVQGRDVLAIMPTGGGKSICYQVPALMLEGLTIVISPLISLMKDQVDTLKDMGVEAEYINSTLSSAEENDIINKIERNEVKILYIAPERLSSSGFLNIIGRCSISQIAVDEAHCISQWGHDFRSSYKKISHFIGILNKRPTVTAFTATASEEVRDDIIRLLKLENPKIFITGFNRENLFINIIKGGDKKNYLLDYINNNSDVSGIIYASTRKEVDNIYELLSSKGYSVVHYHAGLSEEARRENQEGFIYDRANIMVATNAFGMGIDKPDIRYVVHYNMPRNVESYYQEIGRAGRDGDKSECILLFSPQDVQIQKYLIETSIENPERQNIQYKKLQQMIDFVYSNECYKKFILAYFGEELKDDCNSCSNCMNEGEVVDKTVDAQKVLSCIYRMNQKFGSGMVVDVLRGSKNKKVLQFKFNELSTYGIMKEFSADELKTFINTLVSQGYIRVVEGTYPILALNSMSRKILVGEEKVMLKEFAAHKKVRENNELFEILKELRKEIAHENNVPPYVIFGDVTLKEMSVKYPVYKEAMLNITGVGELKYSKYGELFENTIRKYSEDNNIKISGEGTLEIITSNNDNDLKLEIKTDDKLYNLLFEARKKWAEKEKNAYPQSIMHMNILKEISGRYPVSIEELKDISGFGPKKIEAYGDEIIEIVNNYILENNIEHVWIQKNKRKVIIDGETRNSDQIAIDMLKENVNIHEVSEKLELSVSTILGYVSEYIKEFGEIKFNINFEEFYNEEEESAILNAINEVGFEKISDLKKALPKYTKYESIRAVILKHCILA

SEQ ID NO: 8 - DNA, 50 bp, Artificial Sequence
  Synthetic construct
  misc_feature: T1 ss DNA target
  misc_feature (1)..(1): 5′-FAM
tatttaaataatttaatatactatacaacctactacctcatataaatttt

SEQ ID NO: 9 - DNA, 50 bp, Artificial Sequence
  Synthetic construct
  misc_feature: T-2 ss DNA target
  misc_feature (1)..(1): 5′-FAM
cagaatggcttggactcaagtgtaaatgtacagggttcagttttgtaata

SEQ ID NO: 10 - RNA, 50 nt, Artificial Sequence
  Synthetic construct
  misc_feature: T-3 ss RNA target
  misc_feature (1)..(1): 5′-FAM
cagaauggcuuggacucaaguguaaauguacaggguucaguuuuguaaua

SEQ ID NO: 11 - DNA, 53 bp, Artificial Sequence
  Synthetic construct
  misc_feature: ss-phiX174 ss DNA target
  misc_feature (1)..(1): 5′-FAM
gtttgctgatgaactaagtcaacctcagcatcaaccttgcgagtcatttcttt

SEQ ID NO: 12 - DNA, 60 bp, Artificial Sequence
  Synthetic construct
  misc_feature: Top oligo, 60nt Anneals to 60 nt Bottom oligo
  misc_feature (1)..(1): 5′-FAM
actcatgcaggcatgcaggaacgcagtcagatttatgtgtcatatagtacgtgattcaag

SEQ ID NO: 13 - DNA, 60 bp, Artificial Sequence
  Synthetic construct
  misc_feature: Bottom oligo, 60nt Anneals to 60nt Top oligo
taacgtattcaagatacctcgtactctgtactgactgcgttcctgcatgcctgcatgagt

SEQ ID NO: 14 - DNA, 60 bp, Artificial Sequence
  Synthetic construct
  misc_feature: Top oligo, 60 nt Anneals to Bottom 1-4 oligos
  misc_feature (1)..(1): 5′-FAM
actcatgcaggcatgcaggatcgcagtcagatttatgtgtcatatagtacgtgattcaag

SEQ ID NO: 15 - DNA, 60 bp, Artificial Sequence
  Synthetic construct
  misc_feature: Bottom olito 1, 60nt For Blunt stubstrate
cttgaatcacgtactatatgacacataaatctgactgcgatcctgcatgcctgcatgagt

SEQ ID NO: 16 - DNA, 60 bp, Artificial Sequence
  Synthetic construct
  misc_feature: Bottom oligo 2, 60 nt For forked substrate
taacgtattcgtactatatgacacataaatctgactgcgatcctgcatgcctgcatgagt

SEQ ID NO: 17 - DNA, 50 bp, Artificial Sequence
  Synthetic construct
  misc_feature: Bottom oligo 3, 50nt For substrate with 10 nt 3′-ss end
gtactatatgacacataaatctgactgcgatcctgcatgcctgcatgagt

SEQ ID NO: 18 - DNA, 70 bp, Artificial Sequence
  Synthetic construct
  misc_feature: Bottom oligo 4, 70nt For substrate with 10 nt 5′-ss end
gtactctgtacttgaatcacgtactatatgacacataaatctgactgcgatcctgcatgcctgcatgagt

SEQ ID NO: 19 - DNA, 80 bp, Artificial Sequence
  Synthetic construct
  misc_feature: 5′--FAM Top oligo, 80 nt Anneals to 80nt Bottom to make blunt end
  misc_feature (1)..(1): 5′--FAM
gctagatagattgacagattactcaagcaggcatgcaggatcgcagtcagatttatgtgtcatatagtacgtgattcaag

SEQ ID NO: 20 - DNA, 80 bp, Artificial Sequence
  Synthetic construct
  misc_feature: Botom oligo, 80 nt Anneals to 80 nt - FAM to make blunt substrate
cttgaatcacgtactatatgacacataaatctgactgcgatcctgcatgcctgcttgagtaatctgtcaatctatctagc

SEQ ID NO: 21 - DNA, 90 bp, Artificial Sequence
  Synthetic construct
  misc_feature: 5′-FAM Top oligo 1, 90 nt Anneals to 80 nt FAM Top to make 10 nt 5′-ss end
  misc_feature (1)..(1): 5′-FAM
gctagatagattgacagattactcaagcaggcatgcaggatcgcagtcagatttatgtgtcatatagtacgtgattcaagtacagagaac

SEQ ID NO: 22 - DNA, 90 bp, Artificial Sequence
  Synthetic construct
  misc_feature: Bottom olito 1 90.nt Anneals to 90 nt Bottom 2 to make forked end
gttctctgtacttgaatcacgtactatatgacacataaatctgactgcgatcctgcatgcctgcttgagtaatctgtcaatctatctagc

SEQ ID NO: 23 - DNA, 90 bp, Artificial Sequence; identical to SEQ ID NO: 21
  Synthetic construct
  misc_feature: 5′-FAM Top oligo 2, 90 nt Anneals to 90 nt Bottom 2 to make forked end
  misc_feature (1)..(1): 5′-FAM

SEQ ID NO: 24 - DNA, 90 bp, Artificial Sequence
  Synthetic construct
  misc_feature: Bottom oligo 2, 90 nt Anneals to 90 nt FAM Top 2 to make forked end
taaggtattccttgaatcacgtactatatgacacataaatctgactgcgatcctgcatgcctgcttgagtaatctgtcaatctatctagc

SEQ ID NO: 25 - DNA, 17 bp, Artificial Sequence
  Synthetic construct
  misc_feature: B12 DNA Guide
  misc_feature (1)..(1): 5′/phos/
tgactgcgatcctgcat

SEQ ID NO: 26 - DNA, 17 bp, Artificial Sequence
  Synthetic construct
  misc_feature: 17 nt guide DNA guide
  misc_feature (1)..(1): 5′/phos/
tgactgcgttcctgcat

SEQ ID NO: 27 - DNA, 21 bp, Artificial Sequence
  Synthetic construct
  misc_feature: G-1 DNA guide
  misc_feature (1)..(1): 5′/phos/
tgaggtagtaggttgtatagt

SEQ ID NO: 28 - DNA, 17 bp, Artificial Sequence
  Synthetic construct
  misc_feature: G-2 DNA Guide
  misc_feature (1)..(1): 5′/phos/
tgaaccctgtacattta

SEQ ID NO: 29 - RNA, 17 nt, Artificial Sequence
  Synthetic construct
  misc_feature: G-3 RNA guide
  misc_feature (1)..(1): 5′/phos/
ugaacccuguacauuua

SEQ ID NO: 30 - DNA, 21 bp, Artificial Sequence
  Synthetic construct
  misc_feature: T1 DNA guide
  misc_feature (1)..(1): 5′/phos/
tcgcaaggttagtgctgaggt

SEQ ID NO: 31 - DNA, 21 bp, Artificial Sequence
  Synthetic construct
  misc_feature: T1-C1 DNA guide
  misc_feature (1)..(1): 5′/phos/
cgcaaggttagtgctgaggtt

SEQ ID NO: 32 - DNA, 21 bp, Artificial Sequence
  Synthetic construct
  misc_feature: T1-G1 DNA guide
  misc_feature (1)..(1): 5′/phos/
gcaaggttagtgctgaggttg

SEQ ID NO: 33 - DNA, 21 bp, Artificial Sequence
  Synthetic construct
  misc_feature: T1-C2 DNA guide
  misc_feature (1)..(1): 5′/phos/
caaggttagtgctgaggttga

SEQ ID NO: 34 - DNA, 21 bp, Artificial Sequence
  Synthetic construct
  misc_feature: T1-A1 DNA guide
  misc_feature (1)..(1): 5′/phos/
aaggttagtgctgaggttgac

SEQ ID NO: 35 - DNA, 21 bp, Artificial Sequence
  Synthetic construct
  misc_feature: T1-A2 DNA guide
  misc_feature (1)..(1): 5′/phos/
aggttagtgctgaggttgact

SEQ ID NO: 36 - DNA, 21 bp, Artificial Sequence
  Synthetic construct
  misc_feature: T1-G2 DNA guide
  misc_feature (1)..(1): 5′/phos/
ggttagtgctgaggttgactt

SEQ ID NO: 37 - DNA, 21 bp, Artificial Sequence
  Synthetic construct
  misc_feature: T1-G3 DNA guide
  misc_feature (1)..(1): 5′/phos/
gttagtgctgaggttgactta

SEQ ID NO: 38 - DNA, 21 bp, Artificial Sequence
  Synthetic construct
  misc_feature: T2 DNA guide
  misc_feature (1)..(1): 5′/phos/
ttagtgctgaggttgacttag

SEQ ID NO: 39 - DNA, 21 bp, Artificial Sequence
  Synthetic construct
  misc_feature: T2-T2 DNA guide
  misc_feature (1)..(1): 5′/phos/
tagtgctgaggttgacttagt

SEQ ID NO: 40 - DNA, 21 bp, Artificial Sequence
  Synthetic construct
  misc_feature: T2-A1 DNA guide
  misc_feature (1)..(1): 5′/phos/
agtgctgaggttgacttagtt

SEQ ID NO: 41 - DNA, 21 bp, Artificial Sequence
  Synthetic construct
  misc_feature: T2-G1 DNA guide
  misc_feature (1)..(1): 5′/phos/
gtgctgaggttgacttagttc

SEQ ID NO: 42 - DNA, 21 bp, Artificial Sequence
  Synthetic construct
  misc_feature: T3 DNA guide
  misc_feature (1)..(1): 5′/phos/
tgctgaggttgacttagttca

SEQ ID NO: 43 - DNA, 21 bp, Artificial Sequence
  Synthetic construct
  misc_feature: B1 DNA guide
  misc_feature (1)..(1): 5′/phos/
tgaactaagtcaacctcagca

SEQ ID NO: 44 - DNA, 21 bp, Artificial Sequence
  Synthetic construct
  misc_feature: B2 DNA guide
  misc_feature (1)..(1): 5′/phos/
taagtcaacctcagcactaac

SEQ ID NO: 45 - DNA, 21 bp, Artificial Sequence
  Synthetic construct
  misc_feature: B3 DNA guide
  misc_feature (1)..(1): 5′/phos/
tcaacctcagcactaaccttg

SEQ ID NO: 46 - DNA, 21 bp, Artificial Sequence
  Synthetic construct
  misc_feature: B4 DNA guide
  misc_feature (1)..(1): 5′/phos/
tcagcactaaccttgcgagtc

SEQ ID NO: 47 - DNA, 21 bp, Artificial Sequence
  Synthetic construct
  misc_feature: XhoT DNA guide
  misc_feature (1)..(1): 5′/phos/
tttggtctaactttaccgcta

SEQ ID NO: 48 - DNA, 21 bp, Artificial Sequence
  Synthetic construct
  misc_feature: XhoB DNA guide
  misc_feature (1)..(1): 5′/phos/
ttagtagcggtaaagttagac

SEQ ID NO: 49 - DNA, 21 bp, Artificial Sequence
  Synthetic construct
  misc_feature: Sap21F DNA guide
  misc_feature (1)..(1): 5′/phos/
tggtcagattggtcgtcttat

SEQ ID NO: 50 - DNA, 21 bp, Artificial Sequence
  Synthetic construct
  misc_feature: SapB DNA guide
  misc_feature (1)..(1): 5′/phos/
taagacgaccaatctgaccag

SEQ ID NO: 51 - DNA, 21 bp, Artificial Sequence
  Synthetic construct
  misc_feature: StuT DNA guide
  misc_feature (1)..(1): 5′/phos/
tgatggaactgaccaaacgtc

SEQ ID NO: 52 - DNA, 21 bp, Artificial Sequence
  Synthetic construct
  misc_feature: StuB DNA guide
  misc_feature (1)..(1): 5′/phos/
taacgacgtttggtcagttcc

SEQ ID NO: 53 - DNA, 21 bp, Artificial Sequence
  Synthetic construct
  misc_feature: AdB-450T DNA guide
  misc_feature (1)..(1): 5′/phos/
tcaagcaagtgtcttgctgtc

SEQ ID NO: 54 - DNA, 21 bp, Artificial Sequence
  Synthetic construct
  misc_feature: AdB-450B DNA guide
  misc_feature (1)..(1): 5′/phos/
taaagacagcaagacacttgc

SEQ ID NO: 55 - DNA, 21 bp, Artificial Sequence
  Synthetic construct
  misc_feature: AdA-20860T DNA guide
  misc_feature (1)..(1): 5′/phos/
tgtcatcttttaagcttgcct

SEQ ID NO: 56 - DNA, 21 bp, Artificial Sequence
  Synthetic construct
  misc_feature: AdA-20860B DNA guide
  misc_feature (1)..(1): 5′/phos/
taccaggcaagcttaaaagat

SEQ ID NO: 57 - DNA, 21 bp, Artificial Sequence
  Synthetic construct
  misc_feature: T5 DNA guide
  misc_feature (1)..(1): 5′/phos/
taggtatctcagttcggtgta

SEQ ID NO: 58 - DNA, 21 bp, Artificial Sequence
  Synthetic construct
  misc_feature: B5 DNA guide
  misc_feature (1)..(1): 5′/phos/
aacgacctacaccgaactgag

SEQ ID NO: 59 - DNA, 25 bp, Artificial Sequence
  Synthetic construct
  misc_feature: 239-Fw PCR primer for 239 bp substrate, 25 nt
gccaaatgcttactcaagctcaaac

SEQ ID NO: 60 - DNA, 25 bp, Artificial Sequence
  Synthetic construct
  misc_feature: 239-ROX-Fw PCR primer for 239bp substrate, 25 nt
  misc_feature (1)..(1): 5′/ROX/
gccaaatgcttactcaagctcaaac

SEQ ID NO: 61 - DNA, 24 bp, Artificial Sequence
  Synthetic construct
  misc_feature: 239-FAM-Rv PCR primer for 239bp substrate, 24 nt
  misc_feature (1)..(1): 5′/FAM/
tccaagtatcggcaacagctttat

SEQ ID NO: 62 - DNA, 30 bp, Artificial Sequence
  Synthetic construct
  misc_feature: 619-Fw PCR primer for 619bp substrate, 30 nt
ctaaaatgcaactggacaatcagaaagaga

SEQ ID NO: 63 - DNA, 30 bp, Artificial Sequence; identical to SEQ ID NO: 62
  Synthetic construct
  misc_feature: 619-ROX-Fw PCR primer for 619bp substrate, 30 nt
  misc_feature (1)..(1): 5′/ROX/

SEQ ID NO: 64 - DNA, 29 bp, Artificial Sequence
  Synthetic construct
  misc_feature: 619-FAM-Rv PCR primer for 619bp substrate, 29 nt
  misc_feature (1)..(1): 5′/FAM/
tgacattcagaagggtaataagaacgaac

SEQ ID NO: 65 - DNA, 23 bp, Artificial Sequence
  Synthetic construct
  misc_feature: 322-Fw PCR primer for 322bp substrate, 23 nt
  misc_feature (1)..(1): 5′/ROX/
cagaaggagtctactgctcgcgt

SEQ ID NO: 66 - DNA, 24 bp, Artificial Sequence
  Synthetic construct
  misc_feature: 322-Rv PCR primer for 322bp substrate, 24 nt
  misc_feature (1)..(1): 5′/FAM/
tccaagtatcggcaacagctttat

SEQ ID NO: 67 - DNA, 23 bp, Artificial Sequence
  Synthetic construct
  misc_feature: phiX174-ROX-T1-5 PCR primer for 332bp DNA1, 23nt
  misc_feature (1)..(1): 5′/ROX/
atgactcgcaaggttagtgctga

SEQ ID NO: 68 - DNA, 29 bp, Artificial Sequence
  Synthetic construct
  misc_feature: phiX174--FAM-Rv PCR primer for 332bp DNA1, 29 nt
  misc_feature (1)..(1): 5′/FAM/
tgacattcagaagggtaataagaacgaac

SEQ ID NO: 69 - DNA, 25 bp, Artificial Sequence
  Synthetic construct
  misc_feature: pUC19-300-Fw PCR primer for 300bp DNA2, 25nt
  misc_feature (1)..(1): 5′/ROX/
acatgtgagcaaaaggccagcaaaa

SEQ ID NO: 70 - DNA, 23 bp, Artificial Sequence
  Synthetic construct
  misc_feature: pUC19-300-Rv PCR primer for 300bp DNA2, 23nt
  misc_feature (1)..(1): 5′/FAM/
agcgaacgacctacaccgaactg

SEQ ID NO: 71 - DNA, 22 bp, Artificial Sequence
  Synthetic construct
  misc_feature: pET28c-Fw4 PCR primer for pET28c fragment, 22 nt
tgagatccggctgctaacaaag

SEQ ID NO: 72 - DNA, 21 bp, Artificial Sequence
  Synthetic construct
  misc_feature: pET28c-Rv PCR primer for pET28c fragment, 21 nt
catatggctgccgcgcggcac

SEQ ID NO: 73 - DNA, 34 bp, Artificial Sequence
  Synthetic construct
  misc_feature: B1-nhis-Fw PCR primer for recB B1 fragment,34 nt
gcgcggcagccatatgagtgatgtcgccgagaca

SEQ ID NO: 74 - DNA, 27 bp, Artificial Sequence
  Synthetic construct
  misc_feature: B1-A1020-Rv PCR primer for recB B1 fragment, 27 nt
aactccatcgccacctgtttattgcgg

SEQ ID NO: 75 - DNA, 29 bp, Artificial Sequence
  Synthetic construct
  misc_feature: B2-A1020-Fw PCR primer for recB B2 fragment, 29 nt
acaggtggcgatggagttttatctgccga

SEQ ID NO: 76 - DNA, 32 bp, Artificial Sequence
  Synthetic construct
  misc_feature: B2-A1080-Fw PCR primer for recB B2 fragment, 32 nt
tgctcgcctatgcatccaactggttgggtgaa

SEQ ID NO: 77 - DNA, 33 bp, Artificial Sequence
  Synthetic construct
  misc_feature: B3-A1080-Rv PCR primer for recB B3 fragment, 33 nt
ttggatgcataggcgagcaggtaataacgccct

SEQ ID NO: 78 - DNA, 39 bp, Artificial Sequence
  Synthetic construct
  misc_feature: B3-end-Rv PCR primer for recB B3 fragment, 39 nt
gttagcagccggatctcattacgcctcctccagggtcat

SEQ ID NO: 79 - DNA, 60 bp, Artificial Sequence; identical to SEQ ID NO: 14
  Synthetic construct
  misc_feature: Top strand, 60 nt Anneals to bottom strand
  misc_feature (1)..(1): 5′/FAM/
  misc_feature (20)..(20): m6A

SEQ ID NO: 80 - DNA, 60 bp, Artificial Sequence
  Synthetic construct
  misc_feature: Bottom strand, 60 nt Anneals to top strand
taacgtattcaagatacctcgtactctgtactgactgcgatcctgcatgcctgcatgagt

SEQ ID NO: 81 - DNA, 30 bp, Artificial Sequence
  Synthetic construct
  misc_feature: Trap oligo, 30 nt Anneals to top strand
  misc_feature (10)..(10): m6A
ctgactgcgatcctgcatgcctgcatgagt

SEQ ID NO: 82 - DNA, 37 bp, Artificial Sequence
  Synthetic construct
  misc_feature: Top linker oligo 37 nt Anneals to bottom linker oligo
  misc_feature (1)..(1): 5′/phos
gtaggtcgttcgctatgactcgcaaggttagtgctga

SEQ ID NO: 83 - DNA, 37 bp, Artificial Sequence
  Synthetic construct
  misc_feature: Bottom linker oligo, 37 nt Anneals to top linker oligo
  misc_feature (1)..(1): 5′/phos/
aaccttgcgagtcatagcgaacgacctacaccgaact

SEQ ID NO: 84 - DNA, 53 bp, Artificial Sequence
  Synthetic construct
aaagaaatgactcgcaaggttagtgctgaggttgacttagttcatcagcaaac

SEQ ID NO: 85 - DNA, 53 bp, Artificial Sequence; identical to SEQ ID NO: 84
  Synthetic construct
  misc_feature (1)..(1): 5′/ROX/

SEQ ID NO: 86 - DNA, 39 bp, Artificial Sequence
  Synthetic construct
  misc_feature (1)..(1): 5′/ROX/
gactcgcaaggttagtgctgaggttgacttagttcatca

SEQ ID NO: 87 - DNA, 39 bp, Artificial Sequence
  Synthetic construct
  misc_feature (1)..(1): 5′/FAM/
tgatgaactaagtcaacctcagcactaaccttgcgagtc

SEQ ID NO: 88 - DNA, 27 bp, Artificial Sequence
  Synthetic construct
  misc_feature (1)..(1): 5′/ROX/
aaggttagtgctgaggttgacttagtt

SEQ ID NO: 89 - DNA, 27 bp, Artificial Sequence
  Synthetic construct
aactaagtcaacctcagcactaacctt

SEQ ID NO: 90 - DNA, 29 bp, Artificial Sequence
  Synthetic construct
  misc_feature (1)..(1): 5′/ROX/
gcaaggttagtgctgaggttgacttagtt

SEQ ID NO: 91 - DNA, 29 bp, Artificial Sequence
  Synthetic construct
aactaagtcaacctcagcactaaccttgc

SEQ ID NO: 92 - DNA, 30 bp, Artificial Sequence
  Synthetic construct
caaggttagtgctgaggttgacttagttca

SEQ ID NO: 93 - DNA, 30 bp, Artificial Sequence
  Synthetic construct
  misc_feature (1)..(1): 5′/FAM/
tgaactaagtcaacctcagcactaaccttg

SEQ ID NO: 94 - DNA, 20 bp, Artificial Sequence
  Synthetic construct
cgtaattcgtacgaattacg